(19) United States
(12) Patent Application Publication
(10) Pub. No.: US 2020/0289514 A1
(43) Pub. Date: Sep. 17, 2020

(54) Novel Pyrazolo Pyrimidine Derivatives

(71) Applicant: Novartis AG, Basel (CH)

(72) Inventors: Karen KAMMERTOENS, Village-Neuf (FR); Jean QUANCARD, Huningue (FR); Achim SCHLAPBACH, Basel (CH); Oliver SIMIC, Basel (CH); Marina TINTELNOT-BLOMLEY, Maulburg (DE); Grahame WOOLLAM, Basel (CH)

(21) Appl. No.: 15/775,060

(22) PCT Filed: Nov. 11, 2016
(86) PCT No.: PCT/IB2016/056787

§ 371 Date: May 10, 2018

(30) Foreign Application Priority Data
Nov. 13, 2015 (EP) 15194627.4

(51) Int. Cl. A61K 31/519; A61K 45/06; C07D 487/04

(52) U.S. Cl. CPC A61K 31/519; C07B 2200/13; C07D 487/04; A61K 45/06

(57) ABSTRACT

The present invention describes new pyrazolo-pyrimidine derivatives which are generally interacting with MALT1 proteolytic and/or autoproteolytic activity, and in particular which may inhibit said activity. The present invention further describes the synthesis of said new pyrazolo-pyrimidine derivatives, their use as a medicament, especially by interacting with MALT1 proteolytic and/or autoproteolytic activity.

The present invention describes new pyrazolo-pyrimidine derivatives which are generally interacting with MALT1 proteolytic and/or autoproteolytic activity, and in particular which may inhibit said activity. The present invention further describes the synthesis of said new pyrazolo-pyrimidine derivatives, their use as a medicament, especially by interacting with MALT1 proteolytic and/or autoproteolytic activity.

### FIELD OF THE INVENTION

The present invention relates to compounds of formula (I) or pharmaceutically acceptable salts thereof, and to their use in in the treatment of diseases or disorders, in particular susceptible to modulation of proteolytic and/or autoproteolytic activity of MALT1. This may include, but is not limited to autoimmune disorders and inflammatory diseases, such as rheumatoid arthritis, multiple sclerosis, psoriasis, Sjogren's syndrome and systemic lupus erythematosus or vasculitic conditions, cancers of hematopoietic origin or solid tumors, including chronic myelogenous leukemia, myeloid leukemia, non-Hodgkin lymphoma and other B cell lymphomas.

### BACKGROUND OF THE INVENTION

The essential role of MALT1 (mucosa associated lymphoid tissue lymphoma translocation protein 1) in influencing immune responses is described in numerous publications. For example, Rudi Beyaert et al. (WO 2009/065897) describe certain compounds as inhibitors of MALT1 proteolytic and/or autoproteolytic activity.

Studies in BCL10 and MALT 1 deficient mice seem to suggest their essential role in the signaling cascade from the antigen receptors to the transcription factor NFkB. Moreover chromosomal translocations leading to overexpression of BCL10 and MALT 1, or creating the constitutively active fusion protein API2-MALT1, appear to yield in an uncontrolled and stimulus-independent activation of NFkB. Inhibitors of the proteolytic activity of MALT1 have been described with activity in preclinical lymphoma models (Vincendeau et al. Int. J. Hematol. Oncol. 2013, 2, 409).

Moreover, certain publications appear to suggest the important role of MALT1 and its proteolytic function in signaling cascades triggered by innate cell receptors like Dectin receptors and in signaling cascades triggered by G-protein coupled receptors in many cell types.

Consequently, there appears to be a desire to discover and develop potent MALT1 inhibitors comprising valuable pharmacological properties.

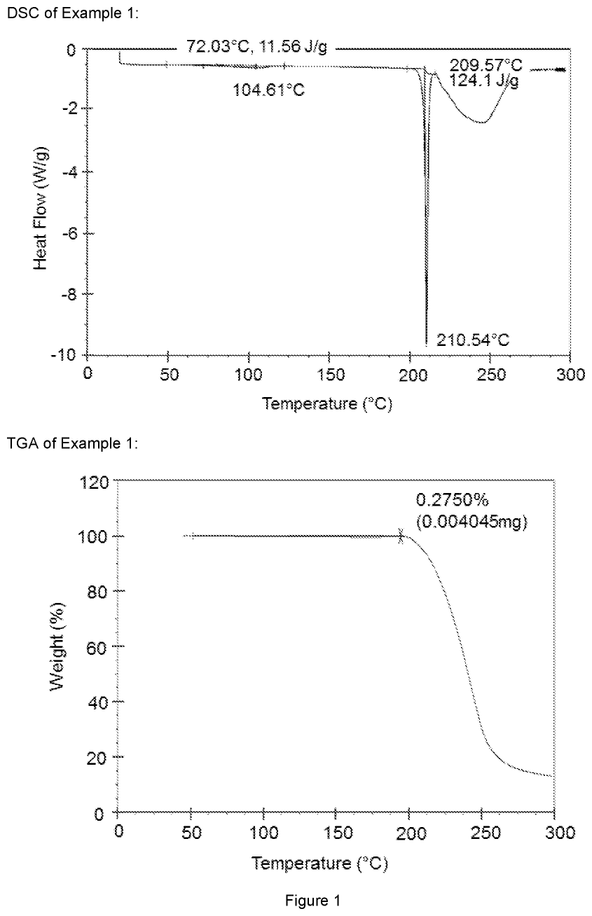

FIG. 1 show the DSC and the TGA of example 1

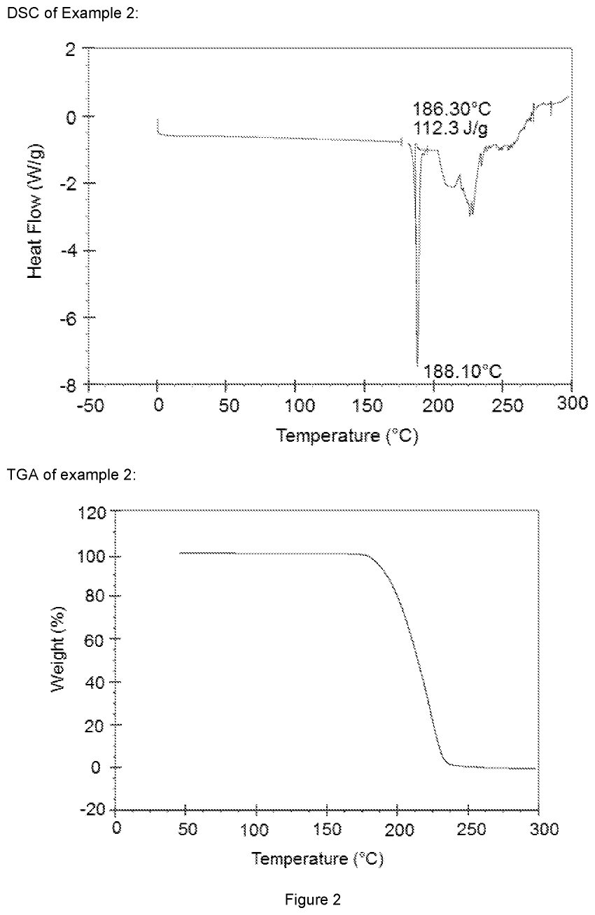

FIG. 2 show the DSC and the TGA of example 2

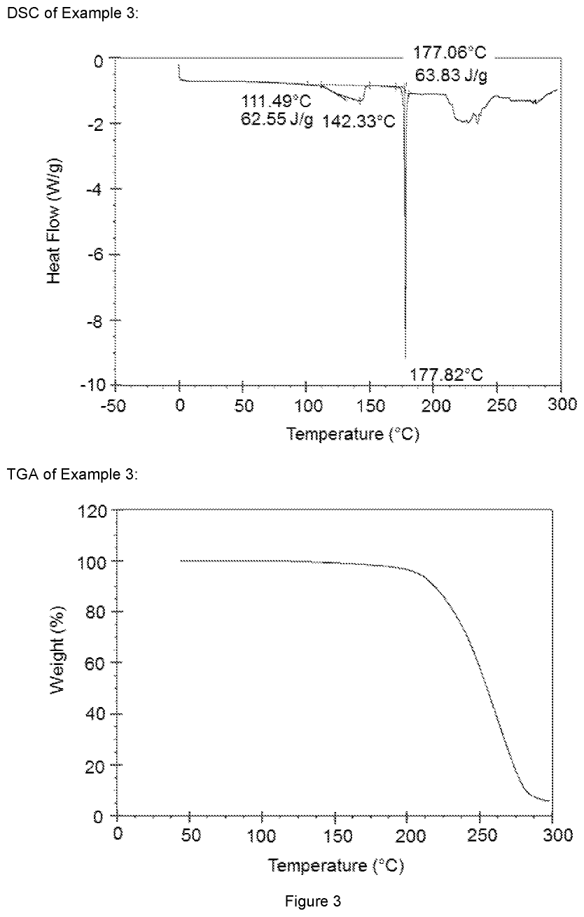

FIG. 3 show the DSC and the TGA of example 3

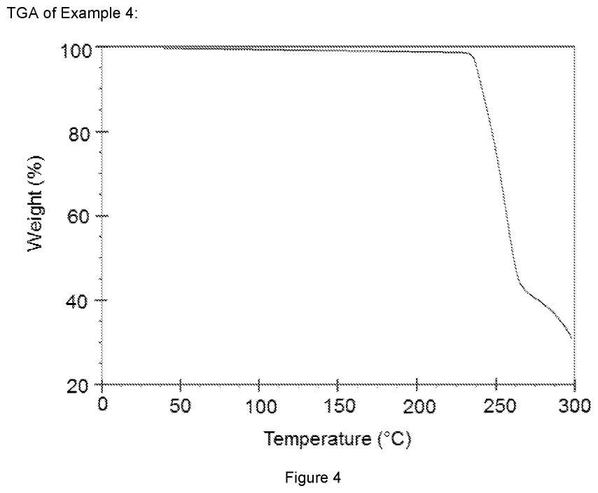

FIG. 4 show the TGA of example 4

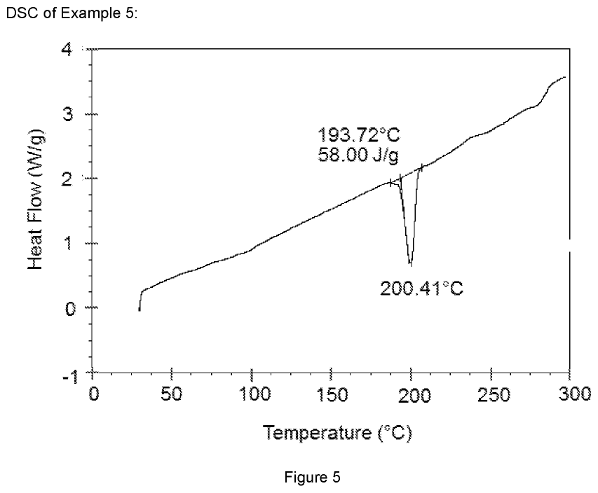

FIG. 5 show the DSC of example 5

### SUMMARY OF THE INVENTION

The present invention describes novel pyrazolo-pyrimidine derivatives according to formula (I) or pharmaceutically acceptable salts thereof as potent inhibitors of MALT1 which may be useful in the treatment of MALT1-related diseases or disorders. This may include, but is not limited to autoimmune disorders and inflammatory diseases, such as rheumatoid arthritis, multiple sclerosis, psoriasis, Sjogren's syndrome and systemic lupus erythematosus or vasculitic conditions. It may further include allergic diseases, airway diseases, such as asthma and chronic obstructive pulmonary disease (COPD) or conditions caused by delayed or immediate type hypersensitivity and anaphylaxis, acute or chronic transplant rejection or graft versus host disease, cancers of hematopoietic origin or solid tumors, including chronic myelogenous leukemia, myeloid leukemia, non-Hodgkin lymphoma and other B cell lymphomas.

### DETAILED DESCRIPTION OF THE INVENTION

In embodiment 1 the present invention provides a compound of formula (I) or a pharmaceutically acceptable salt thereof wherein

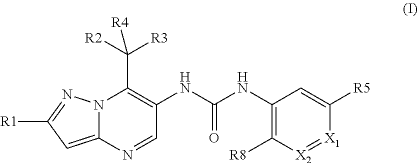

R1 is fluoro, chloro, methyl or cyano;

R2 and R 3 are independently from each other C₁-C₆ alkoxy optionally substituted by C₁-C₆ alkoxy; C₁-C₆ alkyl optionally substituted by halogen or C₁-C₆ alkoxy; amino optionally substituted by C₁-C₆ alkyl; phthalimido; or hydroxy optionally substituted by a 5 or 6 membered heterocyclic ring comprising a nitrogen or oxygen heteroatom wherein said ring is optionally substituted by C₁-C₃ alkyl carbonyl;

or R2 and R3 together with carbon atom to which they are attached form a 3-5 membered carbocyclic ring or heterocyclic ring comprising 1 heteroatom selected from N and O;

R4 is hydrogen; C₁-C₆ alkyl optionally substituted by C₁-C₆ alkoxy;

X₁ is N, N—O or CR6;

X₂ is N or CR7;

R5 is chloro; cyano; or C₁-C₆ alkyl optionally substituted by halogen and/or hydroxy;

R6 is hydrogen; oxo; methoxy; 1,2,3-triazole-2-yl; or aminocarbonyl substituted at the nitrogen atom by R9 and R10;

R7 is hydrogen; C₁-C₆ alkyl optionally substituted by halogen and/or hydroxy; or N,N-dimethylaminocarbonyl;

R8 is hydrogen; C₁-C₆ alkoxy optionally substituted by methoxy or amino;

R9 and 10 are independently of each other hydrogen; C₁-C₆ alkyl optionally substituted by C₁-C₆ alkoxy, N-mono-C₁-C₆ alkyl amino, or N, N-di-C₁-C₆ alkyl amino; or

R9 and 10 together with the nitrogen atom to which they are attached form a 5-7 membered heterocyclic ring having one, two or three ring hetero atoms selected from the group consisting of oxygen, nitrogen and sulphur, that ring being optionally substituted by C₁-C₆ alkyl, hydroxy or oxo;

with the proviso that X₁ and X₂ must not be N at the same time, or X₁ must not be N—O when X₂ is N.

Embodiment 2 relates to a compound of embodiment 1 or a pharmaceutically acceptable salt thereof, wherein

R1 is fluoro or chloro;

R2 is C₁-C₆ alkyl optionally substituted by C₁-C₆ alkoxy;

R3 is C₁-C₆ alkoxy optionally be substituted by C₁-C₆ alkoxy;

R4 is hydrogen;

X₁ is N;

X₂ is CR7;

R5 is chloro; cyano; difluoromethyl; trifluoromethyl;

R7 is hydrogen; and

R8 is hydrogen.

Embodiment 3 relates to a compound of embodiment 1 or a pharmaceutically acceptable salt thereof, wherein

R1 is fluoro or chloro;

R2 is C₁-C₆ alkyl optionally substituted by C₁-C₆ alkoxy;

R3 is C₁-C₆ alkoxy optionally be substituted by C₁-C₆ alkoxy;

R4 is hydrogen;

X₁ is CR6;

X₂ is N;

R5 is chloro; cyano; difluoromethyl; trifluoromethyl;

R6 is hydrogen; oxo; methoxy; 1,2,3-triazole-2-yl; N-methylaminocarbonyl, N,N-dimethylaminocarbonyl; pyrrolidin-1-yl carbonyl and

R8 is hydrogen.

Embodiment 4 relates to a compound of embodiment 1 or a pharmaceutically acceptable salt thereof, wherein

R1 is methyl, fluoro or chloro;

R2 is C₁-C₆ alkyl;

R3 is C₁-C₆ alkoxy;

R4 is hydrogen;

X₁ is CR6;

X₂ is N;

R5 is chloro; cyano; difluoromethyl; trifluoromethyl;

R6 is hydrogen; methoxy; 1,2,3-triazole-2-yl; N-methylaminocarbonyl , N,N-dimethylamino carbonyl; pyrrolidin-1-yl carbonyl and

R8 is hydrogen.

Embodiment 5 relates to a compound of embodiment 1 or a pharmaceutically acceptable salt thereof, wherein

R1 is methyl, fluoro or chloro;

R2 is C₁-C₆ alkyl;

R3 is C₁-C₆ alkoxy;

R4 is hydrogen;

X₁ is N;

X₂ is CR7;

R5 is chloro; cyano; difluoromethyl; trifluoromethyl;

R7 is hydrogen; and

R8 is hydrogen.

Embodiment 6 relates to a compound of embodiment 1 or a pharmaceutically acceptable salt thereof, wherein

R1 is fluoro or chloro;

R2 is C₁-C₆ alkoxy;

R3 is C₁-C₆ alkyl;

R4 is hydrogen;

X₁ is CR6;

X₂ is N;

R5 is chloro; cyano; difluoromethyl; trifluoromethyl;

R6 is hydrogen; methoxy; 1,2,3-triazole-2-yl; N-methylaminocarbonyl , N,N-dimethylamino carbonyl; pyrrolidin-1-yl carbonyl and

R8 is hydrogen.

Embodiment 7 relates to a compound of embodiment 1 or a pharmaceutically acceptable salt thereof, wherein

R1 is fluoro or chloro;

R2 is C₁-C₆ alkoxy;

R3 is C₁-C₆ alkyl;

R4 is hydrogen;

X₁ is N;

X₂ is CR7;

R5 is chloro; cyano; difluoromethyl; trifluoromethyl;

R7 is hydrogen; and

R8 is hydrogen.

Embodiment 8 relates to a compound in particular of embodiment 1 or a pharmaceutically acceptable salt thereof, wherein the compound is selected from (S)-1-(5-cyanopyridin-3-yl)-3-(7-(1-methoxyethyl)-2-methylpyrazolo [1,5-a]pyrimidin-6-yl)urea;

(S)-1-(2-(difluoromethyl)pyridin-4-yl)-3-(2-fluoro-7-(1-methoxyethyl) pyrazolo[1,5-a]pyrimidin-6-yl)urea;

(S)-1-(2-chloro-7-(1-methoxyethyl)pyrazolo[1,5-a]pyrimidin-6-yl)-3-(2-(trifluoromethyl)pyridin-4-yl)urea;

1-(5-chloro-6-(2H-1,2,3-triazol-2-yl)pyridin-3-yl)-3-(2-chloro-7-isopropylpyrazolo[1,5-a]pyrimidin-6-yl)urea;

(S)-1-(5-chloro-6-(2H-1,2,3-triazol-2-yl)pyridin-3-yl)-3-(2-chloro-7-(1-methoxyethyl)pyrazolo[1,5-a]pyrimidin-6-yl)urea;

(S)-1-(5-cyano-6-methoxypyridin-3-yl)-3-(2-fluoro-7-(1-methoxyethyl) pyrazolo[1,5-a]pyrimidin-6-yl)urea;

(S)-1-(6-(2H-1,2,3-triazol-2-yl)-5-(trifluoromethyl)pyridin-3-yl)-3-(2-chloro-7-(1-(2-methoxyethoxy) ethyl)pyrazolo[1,5-a]pyrimidin-6-yl)urea;

(S)-1-(6-(2H-1,2,3-triazol-2-yl)-5-(trifluoromethyl)pyridin-3-yl)-3-(2-chloro-7-(1-methoxy-2-methyl-propyl)pyrazolo[1,5-a]pyrimidin-6-yl)urea;

1-(2-chloro-7-(1-(methoxymethyl)cyclopropyl)pyrazolo[1,5-a]pyrimidin-6-yl)-3-(5-cyanopyridin-3-yl)urea;

1-(5-chloro-6-(2H-1,2,3-triazol-2-yl)pyridin-3-yl)-3-(2-chloro-7-((1R,2S)-1,2-dimethoxypropyl) pyrazolo[1,5-a]pyrimidin-6-yl)urea;

(S)-1-(2-chloro-7-(1-methoxyethyl)pyrazolo[1,5-a]pyrimidin-6-yl)-3-(5-cyano-6-(2H-1,2,3-triazol-2-yl)pyridin-3-yl)urea;

(S)-1-(5-cyanopyridin-3-yl)-3-(2-fluoro-7-(1-methoxyethyl)pyrazolo[1,5-a]pyrimidin-6-yl)urea;

1-(7-((S)-1-(((R)-1-acetylpyrrolidin-3-yl)oxy)ethyl)-2-chloropyrazolo[1,5-a]pyrimidin-6-yl)-3-(5-chloro-6-(2H-1,2,3-triazol-2-yl)pyridin-3-yl)urea; (S)-1-(5-chloro-6-(2H-1,2,3-triazol-2-yl)pyridin-3-yl)-3-(2-fluoro-7-(1-methoxy-2-methylpropyl)-pyrazolo[1,5-a]pyrimidin-6-yl)urea;

(S)-1-(5-cyano-6-(2H-1,2,3-triazol-2-yl)pyridin-3-yl)-3-(7-(1-methoxy-2-methylpropyl)-2-methylpyrazolo[1,5-a]pyrimidin-6-yl)urea;

(S)-1-(2-chloro-7-(1-methoxyethyl)pyrazolo[1,5-a]pyrimidin-6-yl)-3-(5-cyano-6-methoxypyridin-3-yl)urea;

1-(2-fluoro-7-((S)-1-methoxyethyl)pyrazolo[1,5-a]pyrimidin-6-yl)-3-(2-(1-hydroxyethyl)-6-(trifluoromethyl)pyridin-4-yl)urea;

(S)-1-(5-cyano-6-(2H-1,2,3-triazol-2-yl)pyridin-3-yl)-3-(2-fluoro-7-(1-methoxyethyl)pyrazolo[1,5-a]pyrimidin-6-yl)urea;

1-(2-chloro-7-(1,2-dimethoxyethyl)pyrazolo[1,5-a]pyrimidin-6-yl)-3-(5-cyano-6-(2H-1,2,3-triazol-2-yl)pyridin-3-yl)urea;

1-(2-chloro-7-((S)-1-methoxyethyl)pyrazolo[1,5-a]pyrimidin-6-yl)-3-(2-(2,2,2-trifluoro-1-hydroxy-ethyl)pyridin-4-yl)urea;

(S)-1-(5-chloro-2-(2-methoxyethoxy)pyridin-3-yl)-3-(2-chloro-7-(1-methoxyethyl)-pyrazolo[1,5-a]-pyrimidin-6-yl)urea;

(S)-1-(5-cyano-6-methoxypyridin-3-yl)-3-(7-(1-methoxy-2-methylpropyl)-2-methylpyrazolo[1,5-a]-pyrimidin-6-yl)urea;

(S)-1-(2-cyanopyridin-4-yl)-3-(2-fluoro-7-(1-methoxyethyl)pyrazolo[1,5-a]pyrimidin-6-yl)urea; (S)-1-(5-cyano-6-methoxypyridin-3-yl)-3-(7-(1-methoxyethyl)-2-methylpyrazolo[1,5-a]pyrimidin-6-yl)urea;

1-(2-chloro-7-((1R,2S)-1,2-dimethoxypropyl)pyrazolo[1,5-a]pyrimidin-6-yl)-3-(5-cyano-6-(2H-1,2,3-triazol-2-yl)pyridin-3-yl)urea;

1-(7-((S)-1-(((S)-1-acetylpyrrolidin-3-yl)oxy)ethyl)-2-chloropyrazolo[1,5-a]pyrimidin-6-yl)-3-(5-cyano-6-methoxypyridin-3-yl)urea;

(S)-1-(5-cyano-6-(2H-1,2,3-triazol-2-yl)pyridin-3-yl)-3-(7-(1-methoxyethyl)-2-methylpyrazolo[1 ,5-a]pyrimidin-6-yl)urea;

(S)-6-chloro-4-(3-(2-chloro-7-(1-methoxyethyl)pyrazolo[1,5-a]pyrimidin-6-yl)ureido)-N,N-dimethylpicolinamide;

(S)-1-(5-(difluoro-methyl)pyridin-3-yl)-3-(2-fluoro-7-(1-methoxyethyl)-pyrazolo[1,5-a]pyrimidin-6-yl)urea;

(S)-1-(2-fluoro-7-(1-methoxyethyl)pyrazolo[1,5-a]pyrimidin-6-yl)-3-(5-(trifluoro-methyl)pyridin-3-yl)urea;

(S)-3-chloro-5-(3-(2-chloro-7-(1-methoxyethyl)pyrazolo[1,5-a]pyrimidin-6-yl)ureido)-N,N-dimethylpicolinamide;

(S)-1-(5-chloro-pyridin-3-yl)-3-(2-fluoro-7-(1-methoxyethyl)pyrazolo[1,5-a]pyrimidin-6-yl)urea;

(S)-1-(5-chloro-6-(pyrrolidine-1-carbonyl)pyridin-3-yl)-3-(2-chloro-7-(1-methoxyethyl)pyrazolo-[1,5-a]pyrimidin-6-yl)urea

(S)-3-chloro-5-(3-(2-chloro-7-(1-methoxyethyl)pyrazolo[1,5-a]pyrimidin-6-yl)ureido)-N-methylpicolinamide

(S)-1-(2-chloro-7-(1-methoxyethyl)pyrazolo[1,5-a]pyrimidin-6-yl)-3-(5-chloropyridin-3-yl)urea;

(S)-1-(7-(1-aminoethyl)-2-chloropyrazolo[1,5-a]pyrimidin-6-yl)-3-(5-chloro-6-(2H-1,2,3-triazol-2-yl)pyridin-3-yl)urea;

(S)-1-(5-cyanopyridin-3-yl)-3-(7-(1-hydroxyethyl)-2-methylpyrazolo [1,5-a]pyrimidin-6-yl)urea;

(S)-1-(2-(difluoromethyl)pyridin-4-yl)-3-(2-fluoro-7-(1-hydroxyethyl) pyrazolo[1,5-a]pyrimidin-6-yl)urea;

1-(2-((S)-2-aminopropoxy)-5-chloropyridin-3-yl)-3-(2-chloro-7-((S)-1-methoxyethyl)pyrazolo[1,5-a]pyrimidin-6-yl)urea;

(S)-2-(difluoromethyl)-4-(3-(2-fluoro-7-(1-methoxyethyl)pyrazolo[1,5-a]pyrimidin-6-yl)ureido)pyridine 1-oxide;

1-(2-chloro-7-((1R,2S)-1,2-dimethoxypropyl)pyrazolo[1,5-a]pyrimidin-6-yl)-3-(5-cyano-6-methoxypyridin-3-yl)urea;

1-(2-chloro-7-(1-(methoxymethyl)cyclopropyl)pyrazolo[1,5-a]pyrimidin-6-yl)-3-(2-cyanopyridin-4-yl)urea; and

(S)-3-chloro-5-(3-(2-fluoro-7-(1-methoxyethyl)pyrazolo[1,5-a]pyrimidin-6-yl)ureido)picolinamide.

Embodiment 9 relates to a pharmaceutical composition comprising a therapeutically effective amount of a compound according to any one of embodiments 1 to 8 or a pharmaceutically acceptable salt thereof and one or more pharmaceutically acceptable carriers.

Embodiment 10 relates to a combination comprising a therapeutically effective amount of a compound according to any one of embodiments 1 to 8 or a pharmaceutically acceptable salt thereof and one or more therapeutically active co-agents.

Embodiment 11 relates to a method of modulating MALT1 activity in a subject, wherein the method comprises administering to the subject a therapeutically effective amount of a compound according to any one of embodiments 1 to 8 or a pharmaceutically acceptable salt thereof.

Embodiment 12 relates to a compound according to any one of embodiments 1 to 8 or a pharmaceutically acceptable salt thereof, for use as a medicament, in particular for use as a medicament acting as a MALT1 inhibitor.

Embodiment 13 relates to a compound of formula (II) or a pharmaceutically acceptable salt thereof, wherein

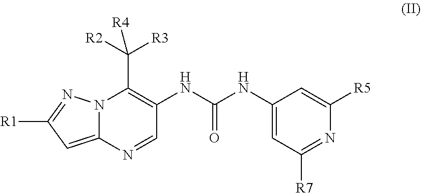

R1 is fluoro or chloro;

R2 and R3 are independently from each other C₁-C₆ alkyl or C₁-C₆ alkoxy;

R4 is hydrogen;

R5 and R7 are independently from each other hydrogen; cyano; halogen or C₁-C₆ alkyl optionally substituted by fluoro and/or hydroxyl.

Embodiment 14 relates to a compound of formula (III) or a pharmaceutically acceptable salt thereof, wherein

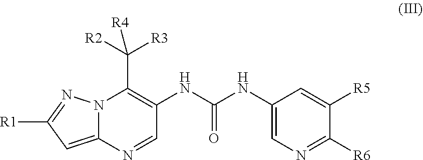

R1 is fluoro or chloro;

R2 and R3 are independently from each other C₁-C₆ alkyl or C₁-C₆ alkoxy;

R4 is hydrogen;

R5 is hydrogen; cyano; halogen or C₁-C₆ alkyl optionally substituted by fluoro and/or hydroxyl; and

R6 is hydrogen; 1,2,3-triazole-2-yl; N,N-dimethylaminocarbonyl; N-monomethylamino carbonyl; or pyrrolidin-1-yl carbonyl.

Embodiment 15 relates to a compound of embodiment 1 or a pharmaceutically acceptable salt thereof, wherein X₁ is N and X₂ is not N, or X₁ is not N and X₂ is N.

Definitions

As used herein DSC stands for differential scanning calorimetry and TGA stands for thermal gravimetric analysis.

As used herein, the term “C₁-C₆ alkyl” refers to a fully saturated branched or unbranched hydrocarbon moiety having up to 6 carbon atoms. Unless otherwise provided, it refers to hydrocarbon moieties having 1 to 6 carbon atoms, 1 to 4 carbon atoms or 1 to 2 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl and the like.

As used herein, the term “C₁-C₆ alkoxy” refers to alkyl-O—, wherein alkyl is defined herein above. Representative examples of alkoxy include, but are not limited to, methoxy, ethoxy, propoxy, 2-propoxy, butoxy, tert-butoxy, pentyloxy, hexyloxy, cyclopropyloxy-, cyclohexyloxy- and the like. Typically, alkoxy groups have about 1 to 6 carbon atoms, 1 to 4 carbon atoms or 1 to 2 carbon atoms.

As used herein, the term “C₁-C₆ alkyl optionally substituted by halogen” refers to C₁-C₆ alkyl as defined above which may be substituted by one or more halogens. Examples include, but are not limited to, trifluoromethyl, difluoromethyl, fluoromethyl, trichloromethyl, 2,2,2-trifluoroethyl, 1-fluoromethyl-2-fluoroethyl, 3-bromo-2-fluoropropyl and 1-bromomethyl-2-bromoethyl.

As used herein, the term “C₁-C₆ alkyl optionally substituted by hydroxyl” refers to C₁-C₆ alkyl as defined above which may be substituted by one or more hydroxy. Examples include, but are not limited to, hydroxymethyl, hydroxyethyl, 1,2-dihydroxyethyl, 2,3-dihyroxy-propyl and the like.

As used herein, the term “di C₁₋₆ alkylamino” refers to a moiety of the formula —N(Rₐ)—Rₐ where each Rₐ is a C₁₋₆alkyl , which may be the same or different, as defined above. In analogy thereto the term “mono C₁₋₆ alkylamino” refers to a moiety of the formula —N(H)—Rₐ where Rₐ is a C₁₋₆alkyl , which may be the same or different, as defined above.

As used herein, the term “halogen” or “halo” refers to fluoro, chloro, bromo, and iodo; and it may in particular refer to chloro; and it may also in particular refer to fluoro.

As used herein, the term “heterocyclyl” or heterocyclic ring refers to a heterocyclic group that is, unless otherwise indicated, saturated or partially saturated and is preferably a monocyclic or a polycyclic ring (in case of a polycyclic ring particularly a bicyclic, tricyclic or spirocyclic ring); and has 3 to 24, more preferably 4 to 16, most preferably 5 to 10 and most preferably 5 or 6 ring atoms; wherein one or more, preferably one to four, especially one or two ring atoms are a heteroatom (the remaining ring atoms therefore being carbon). The bonding ring (i.e. the ring connecting to the molecule) preferably has 4 to 12, especially 5 to 7 ring atoms. The heterocyclic group can be attached at a heteroatom or a carbon atom. The heterocyclyl can include fused or bridged rings as well as spirocyclic rings. Examples of heterocycles include tetrahydrofuran (THF), dihydrofuran, 1, 4-dioxane, morpholine, 1,4-dithiane, piperazine, piperidine, 1,3-dioxolane, imidazolidine, imidazoline, pyrroline, pyrrolidine, tetrahydropyran, dihydropyran, oxathiolane, dithiolane, 1,3-dioxane, 1,3-dithiane, oxathiane, thiomorpholine, and the like.

A substituted heterocyclyl is a heterocyclyl group independently substituted by 1-4, such as one, or two, or three, or four substituents.

As used herein, the term “aryl” refers to an aromatic hydrocarbon group having 6-20 carbon atoms in the ring portion. Typically, aryl is monocyclic, bicyclic or tricyclic aryl having 6-20 carbon atoms. Furthermore, the term “aryl” as used herein, refers to an aromatic substituent which can be a single aromatic ring, or multiple aromatic rings that are fused together. Non-limiting examples include phenyl, naphthyl or tetrahydronaphthyl.

A substituted aryl is an aryl group substituted by 1-5 (such as one, or two, or three) substituents independently selected from the group consisting of hydroxyl, thiol, cyano, nitro, C₁-C₄-alkyl, C₁-C₄-alkenyl, C₁-C₄-alkynyl, C₁-C₄-alkoxy, C₁-C₄-thioalkyl, C₁-C₄-alkenyloxy, C₁-C₄-alkynyloxy, halogen, C₁-C₄-alkylcarbonyl, carboxy, C₁-C₄-alkoxycarbonyl, amino, C₁-C₄-alkylamino, di-C₁-C₄-alkylamino, C₁-C₄-alkylaminocarbonyl, di-C₁-C₄-alkylaminocarbonyl, C₁-C₄-alkylcarbonylamino, C₁-C₄-alkylcarbonyl(C₁-C₄-alkyl)amino, sulfonyl, sulfamoyl, alkylsulfamoyl, C₁-C₄-alkylaminosulfonyl where each of the afore-mentioned hydrocarbon groups (e.g., alkyl, alkenyl, alkynyl, alkoxy residues) may be further substituted by one or more residues independently selected at each occurrence from halogen, hydroxyl or C₁-C₄-alkoxy groups.

As used herein, the terms “salt” or “salts” refers to an acid addition or base addition salt of a compound of the invention. “Salts” include in particular “pharmaceutically acceptable salts”. The term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulfonate, chloride/hydrochloride, chlortheophyllonate, citrate, ethandisulfonate, fumarate, gluceptate, gluconate, glucuronate, hippurate, hydroiodide/iodide, isothionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, stearate, succinate, sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain embodiments, the salts are derived from sodium, potassium, ammonium, calcium, magnesium, iron, silver, zinc, and copper; particularly suitable salts include ammonium, potassium, sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, piperazine and tromethamine.

The pharmaceutically acceptable salts of the present invention can be synthesized from a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH, Weinheim, Germany, 2002).

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁸F, ³¹P, ³²P, ³⁵S, ³⁶Cl, ¹²⁵I respectively. The invention includes various isotopically labeled compounds as defined herein, for example those into which radioactive isotopes, such as ³H and ¹⁴C, or those into which non-radioactive isotopes, such as ²H and ¹³C are present. Such isotopically labeled compounds are useful in metabolic studies (with ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. In particular, an ¹⁸F or labeled compound may be particularly desirable for PET or SPECT studies. Isotopically-labeled compounds of formula (I) can generally be prepared by conventional techniques known to those skilled in the art or by processes analogous to those described in the accompanying Examples and Preparations using an appropriate isotopically-labeled reagents in place of the non-labeled reagent previously employed.

Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent of a compound of the formula (I). The concentration of such a heavier isotope, specifically deuterium, may be defined by the isotopic enrichment factor. The term “isotopic enrichment factor” as used herein means the ratio between the isotopic abundance and the natural abundance of a specified isotope. If a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

Pharmaceutically acceptable solvates in accordance with the invention include those wherein the solvent of crystallization may be isotopically substituted, e.g. D₂O, d₆-acetone, d₆-DMSO.

Compounds of the invention, i.e. compounds of formula (I) that contain groups capable of acting as donors and/or acceptors for hydrogen bonds may be capable of forming co-crystals with suitable co-crystal formers. These co-crystals may be prepared from compounds of formula (I) by known co-crystal forming procedures. Such procedures include grinding, heating, co-subliming, co-melting, or contacting in solution compounds of formula (I) with the co-crystal former under crystallization conditions and isolating co-crystals thereby formed. Suitable co-crystal formers include those described in WO 2004/078163. Hence the invention further provides co-crystals comprising a compound of formula (I).

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drug stabilizers, binders, excipients, disintegration agents, lubricants, sweetening agents, flavoring agents, dyes, and the like and combinations thereof, as would be known to those skilled in the art (see, for example, Remington's Pharmaceutical Sciences, 18th Ed. Mack Printing Company, 1990, pp. 1289-1329). Except insofar as any conventional carrier is incompatible with the active ingredient, its use in the therapeutic or pharmaceutical compositions is contemplated.

The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to (1) at least partially alleviating, inhibiting, preventing and/or ameliorating a condition, or a disorder or a disease (i) mediated by MALT1, or (ii) associated with MALT1 activity, or (iii) characterized by activity (normal or abnormal) of MALT1; or (2) reducing or inhibiting the activity of MALT1; or (3) reducing or inhibiting the expression of MALT1; or (4) modifying the protein levels of MALT1. In another non-limiting embodiment, the term “a therapeutically effective amount” refers to the amount of the compound of the present invention that, when administered to a cell, or a tissue, or a non-cellular biological material, or a medium, is effective to at least partially reducing or inhibiting the activity of MALT1; or reducing or inhibiting the expression of MALT1 partially or completely.

As used herein, the term “subject” refers to an animal. Typically the animal is a mammal. A subject also refers to for example, primates (e.g., humans, male or female), cows, sheep, goats, horses, dogs, cats, rabbits, rats, mice, fish, birds and the like. In certain embodiments, the subject is a primate. In yet other embodiments, the subject is a human.

As used herein, the term “inhibit”, “inhibition” or “inhibiting” refers to the reduction or suppression of a given condition, symptom, or disorder, or disease, or a significant decrease in the baseline activity of a biological activity or process.

As used herein, the term “treat”, “treating” or “treatment” of any disease or disorder refers in one embodiment, to ameliorating the disease or disorder (i.e., slowing or arresting or reducing the development of the disease or at least one of the clinical symptoms thereof). In another embodiment “treat”, “treating” or “treatment” refers to alleviating or ameliorating at least one physical parameter including those which may not be discernible by the patient. In yet another embodiment, “treat”, “treating” or “treatment” refers to modulating the disease or disorder, either physically, (e.g., stabilization of a discernible symptom), physiologically, (e.g., stabilization of a physical parameter), or both. In yet another embodiment, “treat”, “treating” or “treatment” refers to preventing or delaying the onset or development or progression of the disease or disorder.

As used herein, a subject is “in need of” a treatment if such subject would benefit biologically, medically or in quality of life from such treatment.

As used herein, the term “a,” “an,” “the” and similar terms used in the context of the present invention (especially in the context of the claims) are to be construed to cover both the singular and plural unless otherwise indicated herein or clearly contradicted by the context.

All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed.

Any asymmetric atom (e.g., carbon or the like) of the compound(s) of the present invention can be present in racemic or enantiomerically enriched, for example the (R)-, (S)- or (R,S)-configuration. In certain embodiments, each asymmetric atom has at least 50% enantiomeric excess, at least 60% enantiomeric excess, at least 70% enantiomeric excess, at least 80% enantiomeric excess, at least 90% enantiomeric excess, at least 95% enantiomeric excess, or at least 99% enantiomeric excess in the (R)- or (S)-configuration. Substituents at atoms with unsaturated double bonds may, if possible, be present in cis-(Z)- or trans-(E)-form.

Accordingly, as used herein, a compound of the present invention may be in the form of one of the possible rotamers, atropisomers, tautomers or mixtures thereof, or for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure or substantially pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, a basic moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiralstationary phase.

Furthermore, the compounds of the present invention, including their salts, can also be obtained in the form of their hydrates, or include other solvents used for their crystallization. The compounds of the present invention may inherently or by design form solvates with pharmaceutically acceptable solvents (including water); therefore, it is intended that the invention embrace both solvated and unsolvated forms. The term “solvate” refers to a molecular complex of a compound of the present invention (including pharmaceutically acceptable salts thereof) with one or more solvent molecules. Such solvent molecules are those commonly used in the pharmaceutical art, which are known to be innocuous to the recipient, e.g., water, ethanol, and the like. The term “hydrate” refers to the complex where the solvent molecule is water.

The compounds of the present invention, including salts, hydrates and solvates thereof, may inherently or by design form polymorphs.

In another aspect, the present invention provides a pharmaceutical composition comprising a compound of the present invention and a pharmaceutically acceptable carrier. The pharmaceutical composition can be formulated for particular routes of administration such as oral administration, parenteral administration, and rectal administration, etc. In addition, the pharmaceutical compositions of the present invention can be made up in a solid form (including without limitation capsules, tablets, pills, granules, powders or suppositories), or in a liquid form (including without limitation solutions, suspensions or emulsions). The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

Typically, the pharmaceutical compositions are tablets or gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets may contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Certain injectable compositions are aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, or contain about 1-50%, of the active ingredient.

Suitable compositions for transdermal application include an effective amount of a compound of the invention with a suitable carrier. Carriers suitable for transdermal delivery include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays and the like. They are thus particularly suited for use in topical, including cosmetic, formulations well-known in the art. Such may contain solubilizers, stabilizers, tonicity enhancing agents, buffers and preservatives.

As used herein a topical application may also pertain to an inhalation or to an intranasal application. They may be conveniently delivered in the form of a dry powder (either alone, as a mixture, for example a dry blend with lactose, or a mixed component particle, for example with phospholipids) from a dry powder inhaler or an aerosol spray presentation from a pressurised container, pump, spray, atomizer or nebuliser, with or without the use of a suitable propellant.

The present invention further provides anhydrous pharmaceutical compositions and dosage forms comprising the compounds of the present invention as active ingredients, since water may facilitate the degradation of certain compounds.

Anhydrous pharmaceutical compositions and dosage forms of the invention can be prepared using anhydrous or low moisture containing ingredients and low moisture or low humidity conditions. An anhydrous pharmaceutical composition may be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e. g., vials), blister packs, and strip packs.

The invention further provides pharmaceutical compositions and dosage forms that comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

Synthesis of the Compounds of the Present Invention

The synthesis of the compounds of the invention is performed as outlined in Scheme 1:

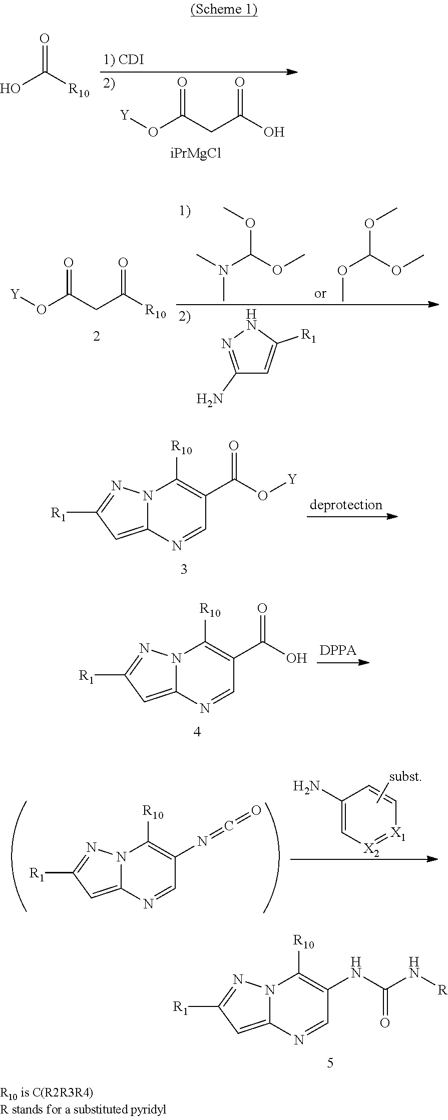

Treatment of an activated acid, e.g. activated as an imidazolid, with the dianion of a malonate mono-ester provides after workup β-ketoester 2. Condensation with a C1 equivalent, e.g. dimethylformamide-dimethylacetal or triethyl orthoformiate, followed by cyclo-condensation with aminopyrazoles in an organic solvent like ethanol at elevated temperature provides the substituted pyrazolo-pyrimidines 3. In case a chiral acid is used in step 1, depending on the substitution pattern, partial racemization may occur during the reaction sequence. In this case the final product may be purified to high enantiomeric purity by chiral chromatography.

Deprotecion of the ester provides acid 4. Curtius rearrangement of acid 4 provides an intermediate isocyanate which is typically reacted with an appropriate aminopyridine derivative in a one pot reaction to form the final product(s).

The synthesis of aminopyrazoles, like 3-amino-5-chloropyrazole can be conducted as follows (Scheme 2):

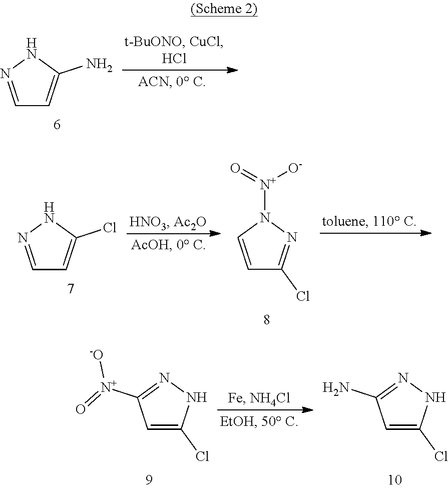

Treatment of aminopyrazole under Sandmeyer conditions provides 3-chloropyrazole. Nitration provides the N-nitropyrazole, which upon heating rearranges to the desired 3-chloro-5-nitropyrazole. Reduction of the nitro group, using iron, tin or tin chloride finally provides the desired 3-amino-5-chloropyrazole 10.

Aminopyridines used in this invention can be prepared using the following route:

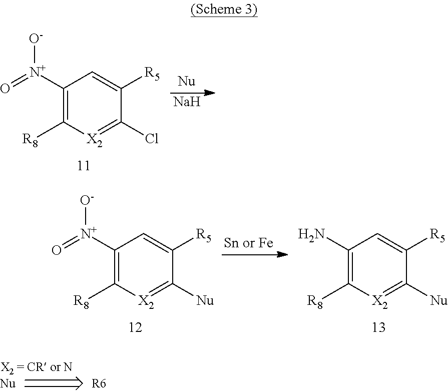

A substituted p-nitrochloropyridine is treated with a nucleophile in an inert solvent like DMF, to give the substitution product 12. The nucleophile in this case can be deprotonated alcohols, amines, lactams or heterocycles, e.g. the anion of 1,2,3 triazole (R6 substituent). Finally reduction of the nitro substituent using tin or iron in acidic media provides the desired aminopyridyl-derivatives 13.

Alternatively, aminopyridines can be prepared via Curtius rearrangement of suitable aryl acids (Scheme 4):

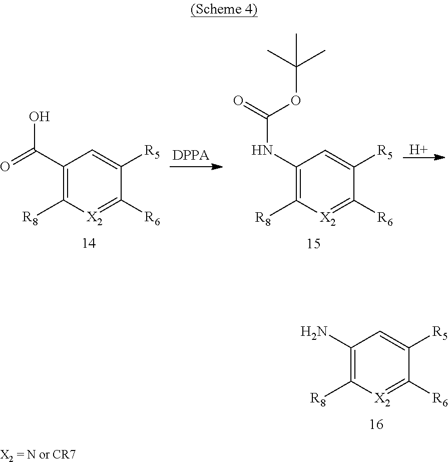

Treatment of acid 14 with diphenyl phosphoryl azide and base in t-butanol provides the t-butoxy-carbonyl-protected amino compound 15, which can be deprotected under acidic conditions using HCl or TFA to give the desired aniline/aminopyridine 16.

Certain aminopyridines and anilines can be prepared by palladium-catalyzed coupling of an aryl halide with a boronic acid according to Scheme 5:

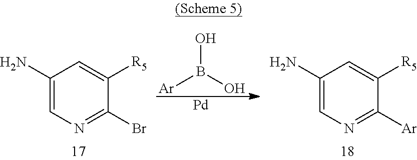

Alkoxypyridines or pyridones of this invention are generally prepared via alkylation of hydroxypyridines (Scheme 6):

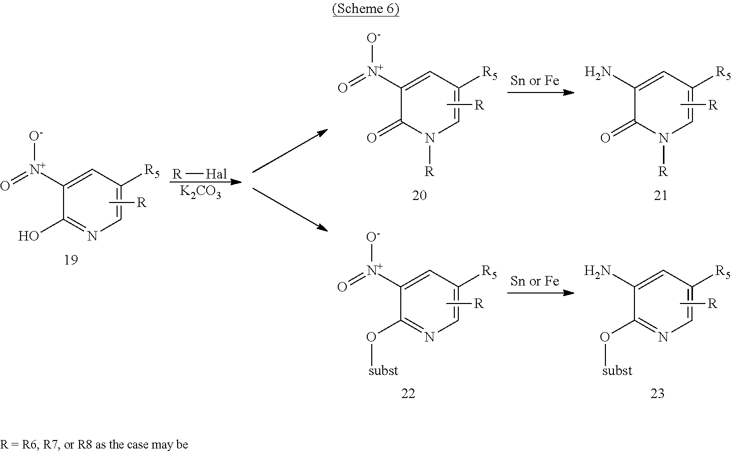

Treatment of a hydroxypyridine 19 with base, e.g. potassium carbonate and an alkylhalide leads to the formation of the pyridone 20 and the alkoxypyridine 22. Depending on the substitution pattern of the reactants selectivity towards one or the other reaction product can be achieved. After separation of the products, each compound can be reduced using standard iron or tin mediated reduction methods to provide the aminopyridones 21, as well as the amino-alkoxypridines 23.

In the Schemes 3-6, 3-nitro-pyridine derivatives are being reacted to yield the appropriate reaction partners for the carboxylic acids 4 shown in scheme 1. In analogy thereto the corresponding 4-nitro-pyrdine derivatives may be obtained in a fully analogous manner.

Furthermore, substituted anilines and amino-pyridines can be obtained from their bromo-analogs by Pd-catalysed amination using an amines source in protected form, like tert-butyl carbamate, followed by deprotecion.

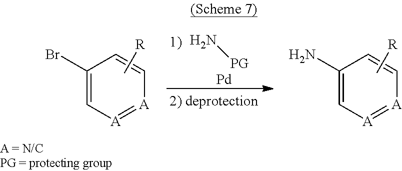

Experimental Section

Abbreviations

Ac₂O acetic anhydride

AcOEt ethyl acetate

AcOH acetic acid

Boc₂O di-tert-butyl dicarbonate

bs broad singulet

n-BuLi n-Butyllithium

CaCl₂ calcium chloride

CCl₄ carbon tetrachloride

CDl carbonyldiimidazole

CHCl₃ chloroform

CH₃CN acetonitrile

CO₂ carbon dioxide

Cs₂CO₃ cesium carbonate

d dublett

DAST diethylamino sulfurtrifluoride

DCE 1,2-dichloroethane

DCM dichloromethane

DEAD (E)-diethyl diazene-1,2-dicarboxylate

DMF dimethylformamide

DMSO dimethylsulfoxide

DPPA diphenyl phosphoryl azide

EDC N-(3-dimethylaminopropyl)-N′-ethylcarbodiimide

Et₂O diethylether

Et₃N triethylamine

EtOH ethanol

h hour

HCl hydrochloric acid

hept. heptett

H₂O water

H₂SO₄ sulfuric acid

HCHO formaldehyde

HCOOH formic acid

HNO₃ nitric acid

HOBt hydroxybenztriazole

HPLC High Performance Liquid Chromatography

HV high vacuum

iPrOH isopropanol

IST International Sorbent Technology (supplier)

K₂CO₃ potassium carbonate

KNO₃ potassium nitroperoxous acid

KOH potassium hydroxyde

l liter

LDA lithium diisopropylamide

LiAlH₄ lithium aluminium hydride

LiCl lithium chloride

LiOH lithium hydroxide

mCPBA meta-chloroperbenzoic acid

MeI methyl iodide

MeOH methanol

MnO₂ manganese dioxide

m multiplett

M molar

min minute

ml milliliter

N normal

NaBH₄ sodium borohydride

NaBH(OAc)₃ sodium triacetoxyborohydride

Na₂CO₃ sodium carbonate

Na₂SO₄ sodium sulfate

NaH sodium hydride

NaHCO₃ sodium bicarbonate

NaIO₄ sodium periodate

NaOH sodium hydroxyde

NH₄Cl ammonium chloride

NMR Nuclear Magnetic Resonance

p pentett

Pd/C palladium on charcoal

PdCl₂(PPh₃)₂ bis(triphenylphosphine)palladium(II) dichloride

Pd₂(dba)₃ tris(dibenzylideneacetone)dipalladium(0)

Pd(PPh₃)₄ tetrakis(triphenylphospine)palladium(0)

pTsOH para-toluenesulfonic acid

q quadruplett

RT room temperature

Rt retention time

s singulet

SFC supercritical fluid chromatography

t triplett

TBME tert-butylmethyl ether

tBuOH tert-butanol

TBAF tetrabutylammonium fluoride

TEA triethylamine

TFA trifluoroacetic acid

THF tetrahydrofuran

UPLC Ultra Performance Liquid Chromatography

XantPhos 4,5-bis(diphenylphosphino)-9,9-dimethylxanthene

Analytical Methods

UPLC Methods

Method B1: Waters UPLC; column: Acquity HSS T3 1.8 μm, 2.1*50 mm, at 60° C., Eluent A: H₂O+0.05% HCOOH+3.75 mM ammonium acetate, B: CH₃CN+0.04% HCOOH, Gradient: 10 to 95% B in 1.5 min, Flow: 1 ml/min.

Method B2: Waters UPLC; column: Acquity HSS T3, 1.8 μm, 2.1*50 mm, at 60° C., Eluent A: H₂O+0.05% HCOOH+3.75 mM ammonium acetate, B: CH₃CN+0.04% HCOOH, Gradient: 5% to 98% B in 1.4 min, Flow: 1 ml/min.

Method B3: Waters UPLC; column: Ascentis Expresse C18 2.1×30 mm, 2.7 μm, at 60° C., Eluent A: H₂O+0.05% TFA, B: CH₃CN+0.04% TFA, Gradient: 2% to 98% B in 1.4 min, Flow: 1 ml/min.

Method B4: Waters UPLC; column: Acquity UPLC BEH C18, 2.1×50 mm, 1.7 μm, at 35° C., Eluent A: H₂O+0.1% TFA, B: CH₃CN+0.1% TFA, Gradient: 5% to 100% B in 1.5 min, Flow: 0.6 ml/min.

Method B5: Waters UPLC; column: Acquity HSS T3, 1.8 μm, 2.1*50 mm, at 50° C., Eluent A: H₂O+0.05% HCOOH+3.75 mM ammonium acetate, B: CH₃CN+0.04% HCOOH, Gradient: 2% to 98% B in 1.4 min, Flow: 1.2 ml/min.

Method B6: Waters UPLC; column: Acquity HSS T3, 1.8 μm, 2.1*50 mm, at 50° C., Eluent A: H₂O+0.05% HCOOH+3.75 mM ammonium acetate, B: CH₃CN+0.04% HCOOH, Gradient: 5% to 98% B in 1.4 min, Flow: 1.2 ml/min.

Method B7: Waters UPLC Acquity; column: Acquity HSS T3, 1.8 μm, 2.1*50 mm, at 60° C., Eluent A: H₂O+0.05% HCOOH+3.75 mM ammonium acetate, B: CH₃CN+0.04% HCOOH, Gradient: 5% to 98% B in 9.4 min, Flow: 1 ml/min.

HPLC Methods

Method C1: Waters X-Bridge C18, 2.5 μm, 3*50 mm, at 40° C., Eluent A: H₂O+0.1% TFA; B: CH₃CN+0.1% TFA. Gradient 10 to 98% B in 8.6 min hold 1.4 min, Flow: 1.4 ml/min.

Method C2: Waters X-Bridge C18, 2.5 μm, 3*30 mm, at 40° C., Eluent A: water+0.1% TFA; B: CH₃CN+0.1% TFA. Gradient 10 to 98% B in 3 min hold 0.5 min, Flow: 1.4 ml/min.

GC/MS Method

Method D1: Gaschromatograph Finnigan Focus GC (Thermo Electron Corporation) Single Quadrupole Mass Analyzer, EI, column Zebron ZB-5 ms, 15 mm, 0.25 mm i.D., 0.25 μm film thickness, 5% polysilarylene, 95% polydimethylsiloxane.

Preparative Methods

Method A1: HPLC, Waters Sunfire C18 OBD, 5 μm, 30*100 mm, Eluent A: H₂O+0.1% TFA, B: CH₃CN+0.1% TFA.

Method A2: HPLC, Waters X-Bridge C18 OBD, 5 μm, 30*100 mm, Eluent A: H₂O+7.3 mM NH₄OH, B: CH₃CN+7.3 mM NH₄OH.

Method A3: Macherey-Nagel Nucleosil 100-10 C18, 5 μm, 40*250 mm, Eluent A: H₂O+0.1% TFA, B: CH₃CN+0.1% TFA.

Method A4: HPLC, Waters X-Bridge C18 OBD, 10 μm, 19*150 mm, Eluent A: H₂O, B: CH₃CN.

Method A5: Thar SFC 200, elution with CO₂/MeOH with one of the following columns:
Princenton PPU 250×30 mm, 100 Å, 5 μm,
Princenton 4-EP 250×30 mm, 60 Å, 5 μm,
Reprosil diNH₂250×30 mm, 100 Å, 5 μm,
Princenton Silica 250×30 mm, 60 Å, 5 μm,
Waters Atlantis Hilic Silica 250×30 mm, 5 μm.

### Part A: Synthesis of Aminopyrazoles

### A1: 5-chloro-1H-pyrazol-3-amine

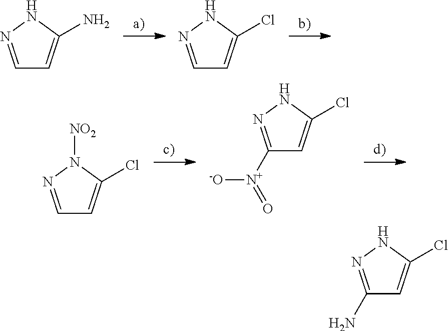

### a) 5-chloro-1H-pyrazole

To a solution of 1H-pyrazol-5-amine (23.6 g, 284 mmol) in CH₃CN (1 L) under a nitrogen atmosphere were added HCl (140 ml, 1420 mmol, 32%) and copper(I) chloride (56.3 g, 568 mmol) at 0° C. Isopentyl nitrite (80 ml, 568 mmol) was added at 0° C. and the mixture was stirred at 0° C. for 2 days. Isopentyl nitrite (20 ml, 0.5 eq) was added and the mixture was stirred at RT for another 5.5 days. The reaction mixture was slowly poured into ammonium hydroxide (1 I, 25%) and extracted with AcOEt. The organic phase was separated and the aqueous phase was extracted with AcOEt. The combined organic layers were washed with brine, dried over Na₂SO₄ and concentrated. The crude product was purified by silica gel column chromatography (hexane/TBME from 1:0 to 4:6) to afford 5-chloro-1H-pyrazole. M/z=103/105 [M+H]+, Rt=0.48 min (UPLC Method B2), ¹H NMR (600 MHz, DMSO-d₆): δ ppm: 13.00 (bs, 1H), 7.79 (t, 1H), 6.29 (t, 1H), isoamyl alcohol: 4.28 (t, 1H), 3.41 (q, 2H), 1.30 (q, 2H), 0.85 (d, 6H).

### b) 5-chloro-1-nitro-1H-pyrazole

To a solution of 5-chloro-1H-pyrazole (3.88 g, 35.2 mmol) in AcOH (5.10 ml, 89 mmol) was added at 0° C. dropwise 90% aqueous HNO₃ (5.10 ml, 35.2 mmol) and the reaction mixture was stirred at 0° C. for 2 h. Ac₂O (12.92 ml, 137 mmol) was then added dropwise. The mixture was stirred at RT for 4 h. The mixture was poured into ice-water and AcOEt and Na₂CO₃ (33.6 g, 317 mmol) were added. The organic phase was separated and the aqueous phase was extracted with AcOEt. The combined organic layers were washed with aqueous saturated NaHCO₃ and brine, dried over Na₂SO₄ and concentrated to afford 5-chloro-1-nitro-1H-pyrazole. M/z=146/148 [M−H]−, Rt=0.71 min (UPLC Method B2), ¹H NMR (400 MHz, DMSO-d₆): δ ppm: 8.91 (d, 1H), 6.90 (d, 1H).

### c) 5-chloro-3-nitro-1H-pyrazole

In an autoclave, 5-chloro-1-nitro-1H-pyrazole (5.44 g, 35.0 mmol) was dissolved in dry anisole (70 ml) and the reactor was sealed. The mixture was heated at 140° C. for 16 h. The mixture was cooled down, filtered and the filtrate was evaporated to dryness. To the residue was added hexane and the suspension was sonicated and triturated. The precipitate was filtered and rinsed with hexane to afford 5-chloro-3-nitro-1H-pyrazole. M/z=146/148 [M−H]−, Rt=0.60 min (UPLC Method B2), ¹H NMR (400 MHz, DMSO-d₆): δ ppm: 7.29 (s, 1H).

### d) 5-chloro-1H-pyrazol-3-amine

To a solution of 5-chloro-3-nitro-1H-pyrazole (4.35 g, 29.2 mmol) in MeOH (389 ml) was added carefully at RT 32% aqueous HCL (57.3 ml, 583 mmol). After cooling to 0° C., SnCl₂ (27.6 g, 146 mmol) was added portionwise and the reaction mixture was stirred at RT overnight. The solvent was evaporated to dryness, the residue was diluted with ethyl acetate and 30% aq. NaOH solution was added until the pH became basic. After cooling to 0° C. overnight, the salts were filtered off through a pad of celite and the cake was rinsed with AcOEt and water. The organic phase was separated and the aqueous phase was extracted with AcOEt. The combined organic layers were washed with brine, dried over Na₂SO₄, filtered and concentrated under vacuum to dryness to afford 5-chloro-1H-pyrazol-3-amine. M/z=118/120 [M+H]+, Rt=0.36 min (UPLC Method B2), ¹H NMR (600 MHz, DMSO-d₆): δ ppm: 11.54 (s, 1H), 5.25 (s, 2H), 5.20 (s, 1H).

### Part B: Synthesis of Carboxylic Acid Compounds

### B1: (S)-2-methoxy-3-methylbutanoic acid

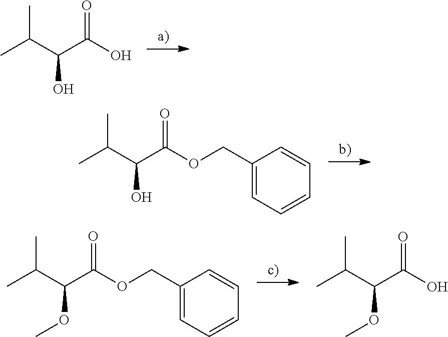

### a) (S)-benzyl 2-hydroxy-3-methylbutanoate

To L-alpha-hydroxyisovaleric acid (4.95 g, 41.9 mmol) in DMF (50 ml) were added benzylbromide (5.95 ml, 50.3 mmol) and DBU (6.32 ml, 41.9 mmol) and the reaction mixture was stirred for 14 h at RT. The solvent was evaporated and the residue was taken up in AcOEt/water. The organic phase was dried over Na₂SO₄, filtered and concentrated. The crude product was purified by flash column chromatography on silica gel (cyclohexane/AcOEt: 1/0 to 9/1) to afford (S)-benzyl 2-hydroxy-3-methylbutanoate. M/z=209 [M+H]+, Rt=0.98 min (UPLC Method B2), ¹H NMR (400 MHz, DMSO-d₆) δ ppm: 7.46-7.29 (m, 5H), 5.35 (d, 1H), 5.14 (d, 2H), 3.87 (dd, 1H), 2.00-1.90 (m, 1H), 0.88 (d, 3H), 0.82 (d, 3H).

### b) (S)-benzyl 2-methoxy-3-methylbutanoate

To (S)-benzyl 2-hydroxy-3-methylbutanoate (8.55 g, 41.1 mmol) in THF (150 ml) at −20° C. was added NaH (1.97 g, 49.3 mmol, 60% oil dispersion) and the mixture was warmed to RT over 30 min. After cooling to 0° C., dimethylsulfate (4.67 ml, 49.3 mmol) was added and the reaction mixture was stirred at RT for 15 h. The mixture was treated with Et₃N, acidified with 1N HCl, the aqueous phase was extracted with TBME and the organic phase washed with brine, dried over Na₂SO₄, filtered and the solvent was evaporated. The residue was purified by flash column chromatography on silica gel (cyclohexane/AcOEt: 1/0 to 9/1) to afford (S)-benzyl 2-methoxy-3-methylbutanoate. M/z=223 [M+H]+, Rt=1.14 min (UPLC Method B2), ¹H NMR (400 MHz, DMSO-d₆) δ 7.47-7.30 (m, 5H), 5.26-5.10 (m, 2H), 3.63 (d, 1H), 3.27 (s, 3H), 2.05-1.90 (m, 1H), 0.88 (d, 3H), 0.84 (d, 3H).

### c) (S)-2-methoxy-3-methylbutanoic acid

To (S)-benzyl 2-methoxy-3-methylbutanoate (2.8 g, 12.8 mmol) in AcOEt (80 ml) was added Pd/C (0.68 g, 10% Pd). The mixture was purged with H₂-gas and the suspension was stirred for 4.5 h at RT. The reaction mixture was filtered, washed with AcOEt and the solvent was evaporated to afford (S)-2-methoxy-3-methylbutanoic acid. M/z=133 [M+H]+, Rt=0.54 min (UPLC Method B2), ¹H NMR (400 MHz, DMSO-d₆) δ ppm: 12.6 (s, 1H), 3.46 (d, 1H), 3.27 (s, 3H), 2.00-1.90 (m, 1H), 0.91 (d, 3H), 0.87 (d, 3H).

### B2: (S)-2-(2-methoxyethoxy)propanoic acid

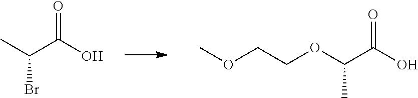

To a suspension of NaH (3.19 g, 80 mmol, 60% oil dispersion) in DMF (60 ml) at 0° C. was added 2-methoxyethanol (2.75 ml, 34.8 mmol). After 30 min, (R)-2-bromopropanoic acid (1.5 ml, 16.6 mmol) was added and the reaction mixture was stirred for 1h at RT. The mixture was quenched with water, concentrated and extracted with AcOEt. The organic phase was dried over Na₂SO₄, filtered and concentrated to afford (S)-2-(2-methoxyethoxy)propanoic acid. ¹H NMR (400 MHz, DMSO-d₆) δ ppm: 12.56 (bs, 1H), 3.92 (q, 1H), 3.66-3.40 (m, 4H), 3.24 (s, 3H), 1.26 (d, 3H).

### B3: (S)-2-(((R)-1-(tert-butoxycarbonyl)pyrolidin-3-yl)oxy)propanoic acid

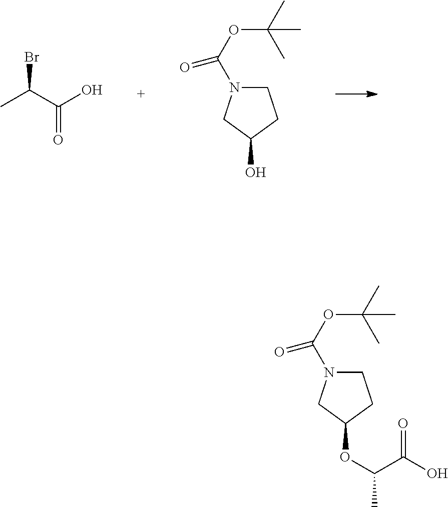

To a suspension of NaH 60% in mineral oil (1.26 g, 31.4 mmol) in dry DMF (20 ml) at 0° C., under argon, was added (R)-tert-butyl 3-hydroxypyrrolidine-1-carboxylate (2.57 g, 13.73 mmol). The reaction mixture was stirred for 30 min at this temperature then (R)-2-bromopropanoic acid (0.591 ml, 6.54 mmol) was added and the reaction mixture was stirred for 3h at RT, quenched with water, concentrated, poured into 1N aq. NaOH and washed with AcOEt. The aqueous layer was then acidified with 1N aq. citric acid to pH 3-4 and extracted several times with AcOEt. The combined organic layers were dried over Na₂SO₄, filtered and concentrated to afford (S)-2-(((R)-1-(tert-butoxycarbonyl)pyrrolidin-3-yl)oxy)propanoic acid which was used in the next step without further purification. ¹H NMR (400 MHz, DMSO-d₆) δ ppm: 12.27 (bs, 1H), 4.11-4.06 (m, 1H), 4.02 (m, 1H), 3.39-3.14 (m, 4H), 1.95-1.77 (m, 2H), 1.39 (s, 9H), 1.25 (d, 3H).

### B4: (S)-2-(((S)-1-(tert-butoxycarbonyl)pyrolidin-3-yl)oxy)propanoic acid

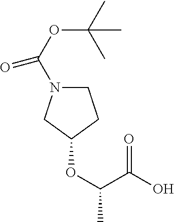 [SMILES: C[C@H](O[C@H]1CCN(C(=O)OC(C)(C)C)C1)C(=O)O]

(S)-2-(((S)-1-(tert-butoxycarbonyl)pyrrolidin-3-yl)oxy)propanoic acid was prepared analogously as described for compound B3 using (S)-tert-butyl 3-hydroxypyrrolidine-1-carboxylate instead of (R)-tert-butyl 3-hydroxypyrrolidine-1-carboxylate. ¹H NMR (400 MHz, DMSO-d₆) δ ppm: 12.61 (bs, 1H), 4.13-4.05 (m, 1H), 3.99 (q, 1H), 3.38-3.18 (m, 4H), 1.93-1.82 (m, 2H), 1.39 (s, 9H), 1.23 (d, 3H).

### B5: 2,3-dimethoxypropanoic acid

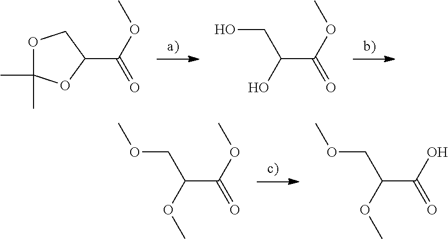

### a) methyl 2,3-dihydroxypropanoate

A solution of methyl 2,2-dimethyl-1,3-dioxolane-4-carboxylate (3 ml, 20.7 mmol) and 1N HCl (25.9 ml, 25.9 mmol) in MeOH (40 ml) was stirred for 20 h at RT. The reaction mixture was extracted with AcOEt, the aqueous phase was extracted with 2-methyltetrahydrofuran, the combined organic phases were dried over Na₂SO₄, filtered and concentrated to afford methyl 2,3-dihydroxypropanoate. ¹H NMR (400 MHz, DMSO-d₆) δ ppm: 5.38 (d, 1H), 4.82 (t, 1H), 4.08-4.03 (m, 1H), 3.62 (s, 3H), 3.57-3.52 (m, 2H).

### b) methyl 2,3-dimethoxypropanoate

A solution of methyl 2,3-dihydroxypropanoate (500 mg, 4.16 mmol), methyl iodide (5.21 ml, 83 mmol) and silver oxide (9.65 g, 41.6 mmol) in DCM (10 ml) was stirred overnight at RT. Water was added and the mixture was extracted with AcOEt, dried over Na₂SO₄, filtered and concentrated. The residue was purified by flash column chromatography on silica gel (cyclohexane/AcOEt 1/0 to 0/1) to afford methyl 2,3-dimethoxypropanoate. ¹H NMR (400 MHz, DMSO-d₆) δ ppm: 4.02 (dd, 1H), 3.67 (s, 3H), 3.60-3.51 (m, 2H), 3.30 (s, 3H), 3.24 (s, 3H).

### c) 2,3-dimethoxypropanoic acid

To a solution of methyl 2,3-dimethoxypropanoate (190 mg, 1.28 mmol) in THF (3 ml) was added NaOH (0.96 ml, 1.92 mmol). The reaction mixture was stirred overnight at RT. 1N HCl was added to adjust the pH to 2-3. The mixture was extracted with AcOEt, dried over Na₂SO₄, filtered and concentrated to afford 2,3-dimethoxypropanoic acid. ¹H NMR (400 MHz, DMSO-d₆) δ ppm: 12.74 (bs, 1H), 3.89 (dd, 1H), 3.58-3.50 (m, 2H), 3.29 (s, 3H), 3.24 (s, 3H).

### Part C: Synthesis of Beta-Ketoesters

### C1: (S)-tert-butyl 4-methoxy-3-oxopentanoate

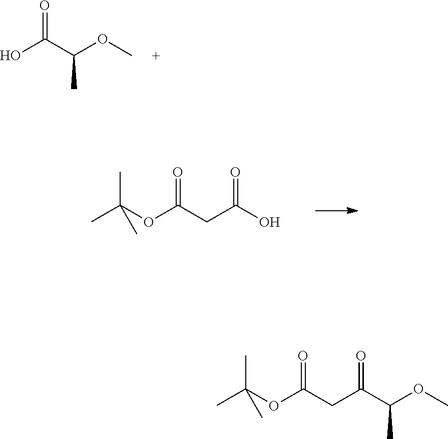

To a solution of (S)-2-methoxypropanoic acid (10.0 g, 96 mmol) in THF (200 ml) at 0° C. was added CDI (17.1 g, 106 mmol) and the reaction mixture was stirred at RT for 3 h. In a separate flask, to a solution of 3-(tert-butoxy)-3-oxopropanoic acid (22.2 ml, 144 mmol) in THF (200 ml) at 0° C. was added dropwise 2M isopropylmagnesium chloride in THF (139 ml, 279 mmol) and the reaction mixture was stirred for 3 h at 20° C. Then, this solution was added dropwise to the acyl imidazole solution at 0° C. and the resulting mixture was stirred for 1 h at RT. The reaction mixture was quenched with 10% aqueous citric acid (25 ml), extracted with AcOEt, washed with aqueous saturated NaHCO₃, dried over Na₂SO₄, filtered and concentrated. The residue was purified by flash column chromatography on silica gel (cyclohexane/AcOEt: 100/0 to 70/30) to afford (S)-tert-butyl 4-methoxy-3-oxopentanoate. M/z=203 [M+H]+, Rt=0.91 min (UPLC Method B1), ¹H NMR (400 MHz, DMSO-d₆) δ ppm: 3.85 (q, 1H), 3.54-3.46 (m, 2H), 3.27 (s, 3H), 1.40 (s, 9H), 1.19 (d, 3H).

In analogy the following ketoesters were prepared:

| Name | Structure | Analytical data |
|---|---|---|
| C2: (R)-tert-butyl 3-(((S)-5- (tert-butoxy)-3,5- dioxopentan-2- yl)oxy)pyrrolidine-1- carboxylate | 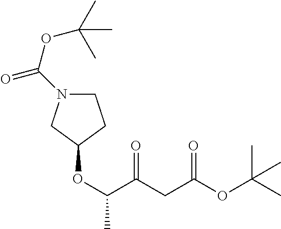 [SMILES: C[C@H](O[C@@H]1CCN(C(=O)OC(C)(C)C)C1)C(=O)CC(=O)OC(C)(C)C] | M/z = 358 [M + H]+, Rt = 1.21 min (UPLC Method B1). |
| C3: (S)-tert-butyl 3-(((S)-5- (tert-butoxy)-3,5- dioxopentan-2- yl)oxy)pyrrolidine-1- carboxylate | 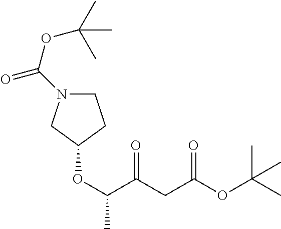 [SMILES: C[C@H](O[C@H]1CCN(C(=O)OC(C)(C)C)C1)C(=O)CC(=O)OC(C)(C)C] | M/z = 356 [M - H]-, Rt = 1.25 min (UPLC Method B1). |
| C4: (S)-tert-butyl 4-(1,3- dioxoisoindolin-2-yl)-3- oxopentanoate | 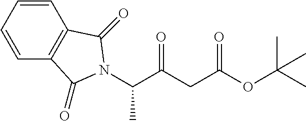 [SMILES: C[C@@H](C(=O)CC(=O)OC(C)(C)C)N1C(=O)c2ccccc2C1=O] | M/z = 316 [M - H]-, Rt = 1.13 min (UPLC Method B1), ¹H NMR (400 MHz, DMSO-d₆) δ ppm: 7.98-7.82 (m, 4H), 4.98 (m, 1H), 3.60 (m, 2H), 1.54 (d, 3H), 1.36 (s, 9H). |
| C5: (S)-tert-butyl 4-(2- methoxyethoxy)-3- oxopentanoate | 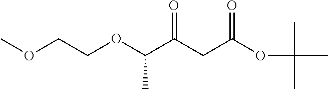 [SMILES: COCCO[C@@H](C)C(=O)CC(=O)OC(C)(C)C] | ¹H NMR (400 MHz, DMSO-d₆) δ ppm: 3.97 (q, 1H), 3.59-3.50 (m, 4H), 3.48-3.41 (m, 2H), 3.25 (s, 3H), 1.40 (s, 9H), 1.20 (d, 3H). |
| C6: tert-butyl 4,5- dimethoxy-3- oxopentanoate | 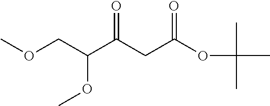 [SMILES: COCC(OC)C(=O)CC(=O)OC(C)(C)C] | ¹H NMR (400 MHz, DMSO-d₆) δ ppm: 3.94 (t, 1H), 3.58 (d, 2H) 3.52-3.42 (m, 2H), 3.34 (s, 3H), 3.23(s, 3H), 1.40 (s, 9H). |
| C7: (S)-tert-butyl 4- methoxy-5-methyl-3- oxohexanoate | 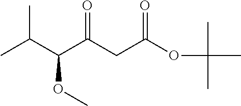 [SMILES: CO[C@H](C(=O)CC(=O)OC(C)(C)C)C(C)C] | ¹H NMR (400 MHz, DMSO-d₆) δ ppm: 3.49 (d, 2H), 3.46-3.42 (m, 1H), 3.29 (s, 3H), 2.00 (pd, 1H), 1.41(d, 9H), 0.88 (d, 3H), 0.84 (d, 3H). |
| C8: tert-butyl 3-(1- (methoxymethyl) cyclopropyl)-3- oxopropanoate | 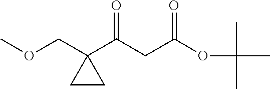 [SMILES: COCC1(C(=O)CC(=O)OC(C)(C)C)CC1] | ¹H NMR (400 MHz, DMSO-d₆) δ ppm: 3.60 (s, 2H), 3.51 (s, 2H), 3.26 (s, 3H), 1.41 (s, 9H), 1.16 (q, 2H), 0.92 (q, 2H). |

### C9: (4S,5S)-tert-butyl 4,5-dimethoxy-3-oxohexanoate

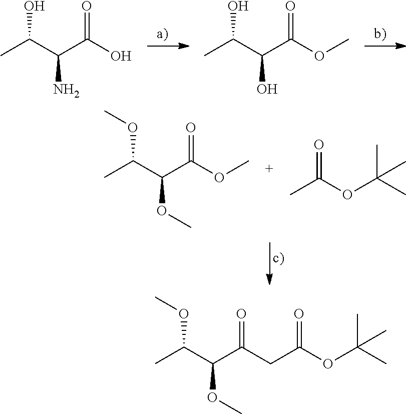

### a) (2S,3S)-methyl 2,3-dihydroxybutanoate

To a solution of L-allo-threonine (5.0 g, 42.0 mmol) in 0.5M aquous H₂SO₄ (91 ml, 45 mmol) was added dropwise at 0° C. a solution of sodium nitrite (9.41 g, 136 mmol) in water (34 ml). The reaction mixture was allowed to warm-up to RT and stirred overnight. The mixture was taken up in MeOH (139 ml) at 0° C. and SOCl₂ (7.60 ml, 104 mmol) was added dropwise. The reaction mixture was allowed to warm-up to RT and stirred for 2 h. The mixture was concentrated and the residue was purified by flash column chromatography on silica gel (cyclohexane/AcOEt 1/0 to 1/1) to afford (2S,3S)-methyl 2,3-dihydroxybutanoate. ¹H NMR (400 MHz, DMSO-d₆) δ ppm: 4.6 (brs, 2H), 3.77 (m, 1H), 3.73 (p, 1H), 3.63 (s, 3H), 1.07 (d, 3H).

### b) (2S,3S)-methyl 2,3-dimethoxybutanoate

A solution of (2S,3S)-methyl 2,3-dihydroxybutanoate (3.0 g, 22.4 mmol), methyl iodide (28.0 ml, 447 mmol) and silver oxide (31.1 g, 134 mmol) in DCM (120 ml) was stirred at RT for 6 days in the dark. The mixture was filtered and concentrated and the crude product was purified by flash column chromatography on silica gel (cyclohexane/AcOEt 1/0 to 1/1) to afford (2S,3S)-methyl 2,3-dimethoxybutanoate. ¹H NMR (400 MHz, DMSO-d₆) δ ppm: 3.85 (d, 1H), 3.69 (s, 3H), 3.53 (p, 1H), 3.30 (s, 3H), 3.25 (s, 3H), 1.09 (d, 3H).

### c) (4S,5S)-tert-butyl 4,5-dimethoxy-3-oxohexanoate

At −78° C., a solution of tert-butyl acetate (2.38 ml, 17.6 mmol) in dry THF (7.4 ml) was added dropwise to a mixture of dry THF (7.4 ml) and 2M LDA in THF/heptane/ethylbenzene (7.72 ml, 15.4 mmol). After 1 h stirring at −78° C., the solution was canulated dropwise to a solution of (2S,3S)-methyl 2,3-dimethoxybutanoate (1.10 g, 4.41 mmol) in dry THF (7.4 ml). The resulting mixture was stirred at −78° C. for 2 h. The reaction mixture was poured into 1M aqueous HCl and extracted with AcOEt, dried over a phase separator cartridge (IST) and evaporated. The crude material was purified by flash column chromatography on silica gel (cyclohexane/AcOEt 1/0 to 9/1) to afford (4S,5S)-tert-butyl 4,5-dimethoxy-3-oxohexanoate. M/z=247 [M+H]+, Rt=1.02 min (UPLC Method B2), ¹H NMR (400 MHz, DMSO-d₆) δ ppm: 3.79 (m, 1H), 3.64-3.57 (m, 1H), 3.48-3.44 (m, 2H), 3.35 (s, 3H), 3.26 (s, 3H), 1.42 (s, 9H), 1.05 (d, 3H).

### Part D: Synthesis of C-substituted pyrazolo[1,5-a]pyrimidine-6-carboxylates

### D1: (S)-2-chloro-7-(1-methoxyethyl)pyrazolo[1,5-a]pyrimidine-6-carboxylic acid

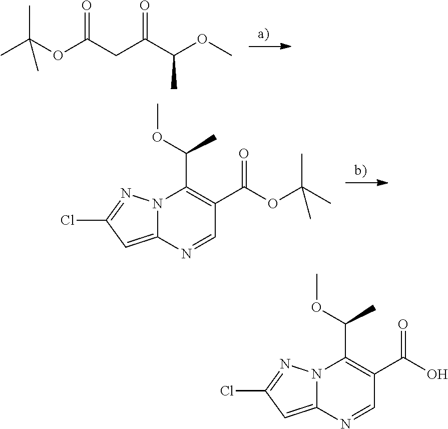

### a) (S)-tert-butyl 2-chloro-7-(1-methoxyethyl)pyrazolo[1,5-a]pyrimidine-6-carboxylate

A mixture of 1,1-dimethoxy-N,N-dimethylmethanamine (12.4 ml, 94 mmol) and (S)-tert-butyl 4-methoxy-3-oxopentanoate (18.9 g, 94 mmol) was stirred at 120° C. for 1.5 h. Then, a solution of 5-chloro-1H-pyrazol-3-amine (11.0 g, 94 mmol) in EtOH (100 ml) was added and the resulting mixture was stirred 1 h at 85° C. The reaction mixture was concentrated and the residue was purified by flash column chromatography on silica gel (cyclohexane/AcOEt: 100/0 to 70/30) to afford (S)-tert-butyl 2-chloro-7-(1-methoxyethyl)pyrazolo[1,5-a]pyrimidine-6-carboxylate. M/z=312-314 [M+H]+, Rt=1.31 min (UPLC Method B1), ¹H NMR (400 MHz, DMSO-d₆) δ ppm: 8.65 (s, 1H), 7.03 (s, 1H), 5.26 (q, 1H), 3.22 (s, 3H), 1.62 (d, 3H), 1.55 (s, 9H).

### b) (S)-2-chloro-7-(1-methoxyethyl)pyrazolo[1,5-a]pyrimidine-6-carboxylic acid

To a solution of (S)-tert-butyl 2-chloro-7-(1-methoxyethyl)pyrazolo[1,5-a]pyrimidine-6-carboxylate (15.0 g, 48.1 mmol) in DCM (75 ml) at RT was added TFA (74 ml). The reaction mixture was stirred overnight and concentrated. Et₂O was added to the residue and the suspension was evaporated to dryness to afford (S)-2-chloro-7-(1-methoxyethyl)pyrazolo[1,5-a]pyrimidine-6-carboxylic acid. M/z=256-258 [M+H]+, Rt=0.57 min (UPLC Method B1), ¹H NMR (400 MHz, DMSO-d₆) δ ppm: 8.72 (s, 1H), 7.03 (s, 1H), 5.40 (q, 1H), 3.20 (s, 3H), 1.64 (d, 3H).

### D2: (S)-2-fluoro-7-(1-methoxy-2-methylpropyl)pyrazolo[1,5-a]pyrimidine-6-carboxylic acid

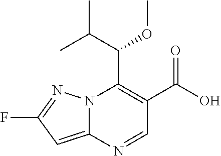 [SMILES: CO[C@H](c1c(C(=O)O)cnc2cc(F)nn12)C(C)C]

(S)-2-fluoro-7-(1-methoxy-2-methylpropyl)pyrazolo[1,5-a]pyrimidine-6-carboxylic acid was prepared analogously as described for compound D1 using (S)-tert-butyl 4-methoxy-5-methyl-3-oxohexanoate and and 5-fluoro-1H-pyrazol-3-amine in step a). M/z=268 [M+H]+, Rt=0.78 min (UPLC Method B2),¹H NMR (400 MHz, DMSO-d₆) δ ppm: 13.77 (bs, 1H), 8.80 (s, 1H), 6.67 (d, 1H), 5.09 (d, 1H), 3.17 (s, 3H), 2.78 (m, 1H), 1.10 (d, 3H), 0.67 (d, 3H).

In analogy the following compounds were prepared:

| Name | Structure | Analytical data |
|---|---|---|
| D3: (S)-7-(1-methoxy-2- methylpropyl)-2- methylpyrazolo[1,5- a]pyrimidine-6- carboxylic acid | 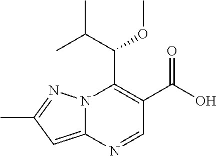 [SMILES: CO[C@H](c1c(C(=O)O)cnc2cc(C)nn12)C(C)C] | M/z = 264 [M + H]+, Rt = 0.79 min (UPLC Method B2), ¹H NMR (400 MHz, DMSO-d₆) δ ppm: 13.8 (bs, 1H), 8.65 (s, 1H), 6.64 (s, 1H), 5.17 (d, 1H), 3.16 (s, 3H), 2.96-2.80 (m, 1H), 2.46 (s, 3H), 1.10 (d, 3H), 0.65 (d, 3H). |
| D4: (S)-2-chloro-7-(1-(2- methoxyethoxy)ethyl) pyrazolo[1,5-a]pyrimidine- 6-carboxylic acid | 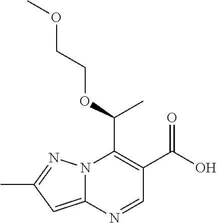 [SMILES: COCCO[C@@H](C)c1c(C(=O)O)cnc2cc(C)nn12] | M/z = 300-302 [M + H]+, Rt = 0.63 min (UPLC Method B1). |
| D5: 2-chloro-7-(1,2- dimethoxyethyl)pyrazolo [1,5-a]pyrimidine-6- carboxylic acid | 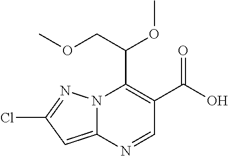 [SMILES: COCC(OC)c1c(C(=O)O)cnc2cc(Cl)nn12] | M/z = 286-288 [M + H]+, Rt = 0.57 min (UPLC Method B1), ¹H NMR (400 MHz, DMSO-d₆) δ ppm: 8.77 (s, 1H), 7.04 (s, 1H), 5.65 (dd, 1H), 4.01 (dd, 1H), 3.79 (dd, 1H), 3.31 (s, 3H), 3.25 (s, 3H). |
| D6: (S)-2-chloro-7-(1- methoxy-2- methylpropyl)pyrazolo[1, 5-a]pyrimidine-6- carboxylic acid | 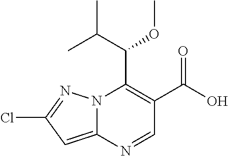 [SMILES: CO[C@H](c1c(C(=O)O)cnc2cc(Cl)nn12)C(C)C] | M/z = 284-286 [M + H]+, Rt = 0.81 min (UPLC Method B2). |
| D7: 2-chloro-7-(1- (methoxymethyl) cyclopropyl)pyrazolo[1,5- a]pyrimidine-6-carboxylic acid | 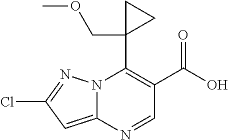 [SMILES: COCC1(c2c(C(=O)O)cnc3cc(Cl)nn23)CC1] | M/z = 282-284 [M+ H]+, Rt = 0.76 min (UPLC Method B2). |
| D8: 2-chloro-7-((1R, 2S)- 1,2-dimethoxypropyl) pyrazolo[1,5-a]pyrimidine- 6-carboxylic acid | 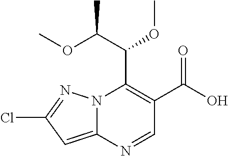 [SMILES: CO[C@@H](C)[C@H](OC)c1c(C(=O)O)cnc2cc(Cl)nn12] | M/z = 300-302 [M + H]+, Rt = 0.75 min (UPLC Method B2), ¹H NMR (400 MHz, DMSO-d₆) δ ppm: 8.81 (d, 1H), 7.02 (d, 1H), 5.45 (d, 1H), 4.31 (m, 1H), 3.25 (s, 3H), 2.99 (s, 3H), 1.29 (d, 3H). |
| D9: (S)-2-chloro-7-(1- (1,3-dioxoisoindolin-2- yl)ethyl)pyrazolo[1,5- a]pyrimidine-6-carboxylic acid | 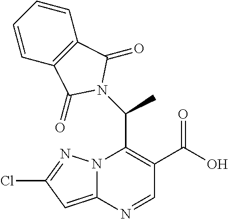 [SMILES: C[C@@H](c1c(C(=O)O)cnc2cc(Cl)nn12)N1C(=O)c2ccccc2C1=O] | [a], M/z = 371-373 [M + H]+, Rt = 0.87 min (UPLC Method B1). |

[a]: HCl 4M in dioxane was used instead of TFA/DCM in step b).

### D10: (S)-7-(1-methoxyethyl)-2-methylpyrazolo[1,5-a]pyrimidine-6-carboxylic acid

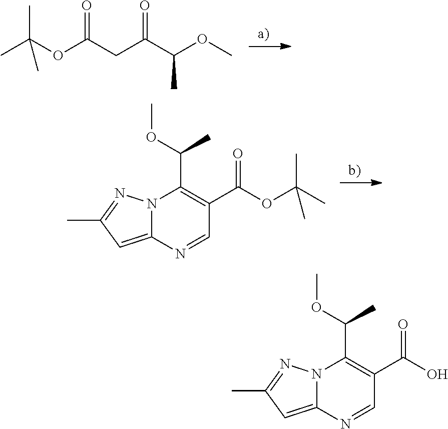

### a) (S)-tert-butyl 7-(1-methoxyethyl)-2-methylpyrazolo[1,5-a]pyrimidine-6-carboxylate

A mixture of 1,1-dimethoxy-N,N-dimethylmethanamine (0.66 ml, 4.94 mmol) and (S)-tert-butyl 4-methoxy-3-oxopentanoate (1.0 g, 4.94 mmol) was stirred at 120° C. for 1 h. Then, a solution of 5-methyl-1H-pyrazol-3-amine (0.48 g, 4.94 mmol) in EtOH (5 ml) was added and the reaction mixture was stirred at 80° C. for 1.5 h. The mixture was concentrated and the crude product was purified by flash column chromatography on silica gel (cyclohexane/AcOEt 1/0 to 8/2) to afford (S)-tert-butyl 7-(1-methoxyethyl)-2-methylpyrazolo[1,5-a]pyrimidine-6-carboxylate. M/z=292 [M+H]+, Rt=1.19 min (UPLC Method B1).

### b) (S)-7-(1-methoxyethyl)-2-methylpyrazolo[1,5-a]pyrimidine-6-carboxylic acid

To a solution of (S)-tert-butyl 7-(1-methoxyethyl)-2-methylpyrazolo[1,5-a]pyrimidine-6-carboxylate (1.07 g, 3.67 mmol) in DCM (5 ml) was added TFA (5.66 ml, 73.5 mmol). The reaction mixture was stirred at RT for 2 days. The mixture was evaporated and the residue was taken-up in Et₂O. The solid was filtered, washed with Et₂O and dried under HV to afford (S)-7-(1-methoxyethyl)-2-methylpyrazolo[1,5-a]pyrimidine-6-carboxylic acid. The filtrate was concentrated and basified with saturated aqueous NaHCO₃ and extracted with AcOEt. The organic layer was dried over Na₂SO₄, filtered and evaporated to afford another batch of (S)-7-(1-methoxyethyl)-2-methylpyrazolo[1,5-a]pyrimidine-6-carboxylic acid. M/z=236 [M+H]+, Rt=0.50 min (UPLC Method B1), ¹H NMR (400 MHz, DMSO-d₆) δ ppm: 13.47 (bs, 1H), 8.58 (s, 1H), 6.65 (s, 1H), 5.46 (q, 1H), 3.19 (s, 3H), 2.47 (s, 3H), 1.65 (d, 3H).

### D11: (S)-2-fluoro-7-(1-methoxyethyl)pyrazolo[1,5-a]pyrimidine-6-carboxylic acid

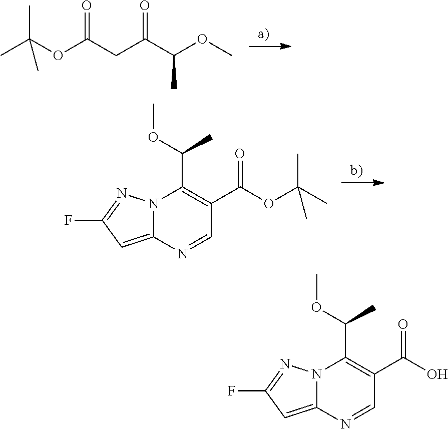

### a) (S)-tert-butyl 2-fluoro-7-(1-methoxyethyl)pyrazolo[1,5-a]pyrimidine-6-carboxylate

A mixture of 1,1-dimethoxy-N,N-dimethylmethanamine (1.31 ml, 9.89 mmol) and (S)-tert-butyl 4-methoxy-3-oxopentanoate (0.40 g, 1.98 mmol) was stirred at 120° C. for 1 h. Then, a solution of 5-fluoro-1H-pyrazol-3-amine (0.30 mg, 2.97 mmol) in EtOH (6.6 ml) was added and the reaction mixture was stirred overnight at 80° C. The mixture was diluted with water and extracted twice with AcOEt. The organic layer was washed with saturated aqueous NaHCO₃, water and brine, dried over a phase separator cartridge (1ST) and evaporated. The crude material was purified by flash column chromatography on silica gel (cyclohexane/AcOEt 1/0 to 9/1) to afford (S)-tert-butyl 2-fluoro-7-(1-methoxyethyl)pyrazolo[1,5-a]pyrimidine-6-carboxylate. M/z=296 [M+H]+, Rt=1.21 min (UPLC Method B2), ¹H NMR (400 MHz, DMSO-d₆) δ ppm: 8.65 (s, 1H), 6.67 (d, 1H), 5.20 (q, 1H), 3.21 (s, 3H), 1.61 (d, 3H), 1.55 (s, 9H).

### b) (S)-2-fluoro-7-(1-methoxyethyl)pyrazolo[1,5-a]pyrimidine-6-carboxylic acid

To a solution of (S)-tert-butyl 2-fluoro-7-(1-methoxyethyl)pyrazolo[1,5-a]pyrimidine-6-carboxylate (0.67 g, 2.25 mmol) in DCM (5 ml) was added TFA (3.47 ml, 45.0 mmol). The reaction mixture was stirred overnight at RT. The mixture was concentrated and the residue was co-evaporated with toluene. The residue was taken-up in Et₂O, the solid was filtered, washed with Et₂O and dried under HV to afford (S)-2-fluoro-7-(1-methoxyethyl)pyrazolo[1,5-a]pyrimidine-6-carboxylic acid. M/z=240 [M+H]+, Rt=0.57 min (UPLC Method B2), ¹H NMR (400 MHz, DMSO-d₆) δ ppm: 13.63 (bs, 1H), 8.73 (s, 1H), 6.67 (d, 1H), 5.37 (q, 1H), 3.19 (s, 3H), 1.63 (d, 3H).

### D12: 7-((S)-1-(((R)-1-acetylpyrrolidin-3-yl)oxy)ethyl)-2-chloropyrazolo[1,5-a]pyrimidine-6-carboxylic acid

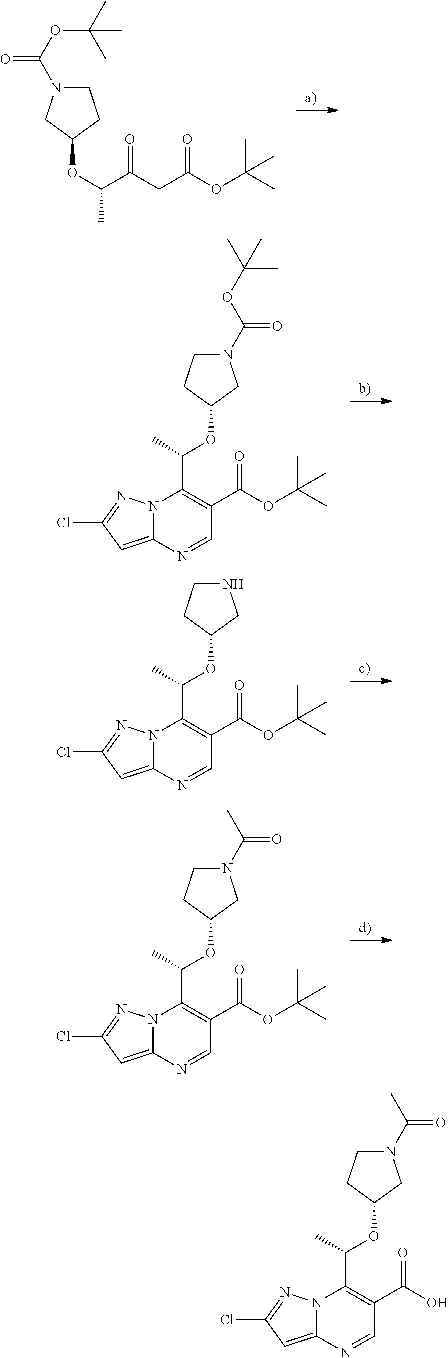

### a) tert-butyl 7-((S)-1-(((R)-1-(tert-butoxycarbonyl)pyrrolidin-3-yl)oxy)ethyl)-2-chloro-pyrazolo[1,5-a]pyrimidine-6-carboxylate

tert-butyl 7-((S)-1-(((R)-1-(tert-butoxycarbonyl)pyrrolidin-3-yl)oxy)ethyl)-2-chloropyrazolo-[1,5-a]pyrimidine-6-carboxylate was prepared analogously as described for compound D1 step a) using (R)-tert-butyl 3-(((S)-5-(tert-butoxy)-3,5-dioxopentan-2-yl)oxy)-pyrrolidine-1-carboxylate instead of (S)-tert-butyl 4-methoxy-3-oxopentanoate. M/z=467-469 [M+H]+, Rt=1.52 min (UPLC Method B1).

### b) tert-butyl 2-chloro-7-((S)-1-((R)-pyrrolidin-3-yloxy)ethyl)pyrazolo[1,5-a]pyrimidine-6-carboxylate

To a solution of tert-butyl 7-((S)-1-(((R)-1-(tert-butoxycarbonyl)pyrrolidin-3-yl)oxy)ethyl)-2-chloropyrazolo[1,5-a]pyrimidine-6-carboxylate (730 mg, 1.56 mmol) in dioxane (2 ml) was added 4N HCl in dioxane (3.91 ml, 15.6 mmol). The reaction mixture was stirred for 1 h at RT, treated at 0° C. with sat. aq. NaHCO₃ and extracted with AcOEt. The organic layer was dried over Na₂SO₄, filtered, concentrated and purified by flash column chromatography on silica gel (DCM/MeOH: 10/0 to 8/2) to afford tert-butyl 2-chloro-7-((S)-1-((R)-pyrrolidin-3-yloxy)ethyl)pyrazolo[1,5-a]pyrimidine-6-carboxylate. M/z=367-369 [M+H]+, Rt=0.80 min (UPLC Method B1).

### c) tert-butyl 7-((S)-1-(((R)-1-acetylpyrrolidin-3-yl)oxy)ethyl)-2-chloropyrazolo[1,5-a]pyrimidine-6-carboxylate

To a solution of tert-butyl 2-chloro-7-((S)-1-((R)-pyrrolidin-3-yloxy)ethyl)pyrazolo[1,5-a]pyrimidine-6-carboxylate (310 mg, 0.85 mmol) in DCM (5 ml) at 0° C., were added TEA (0.353 ml, 2.54 mmol) followed by acetyl chloride (0.090 ml, 1.27 mmol). The reaction mixture was stirred for 1 h at RT, quenched at 0° C. with sat. aq. NaHCO₃ and extracted with AcOEt. The organic layer was dried over Na₂SO₄, filtered, concentrated and purified by flash column chromatography on silica gel (DCM/MeOH: 10/0 to 8/2) to afford tert-butyl 7-((S)-1-(((R)-1-acetylpyrrolidin-3-yl)oxy)ethyl)-2-chloropyrazolo[1,5-a]pyrimidine-6-carboxylate. M/z=409-411 [M+H]+, Rt=1.13 min (UPLC Method B1).

### d) 7-((S)-1-(((R)-1-acetylpyrrolidin-3-yl)oxy)ethyl)-2-chloropyrazolo[1,5-a]pyrimidine-6-carboxylic acid

To a solution of tert-butyl 7-((S)-1-(((R)-1-acetylpyrrolidin-3-yl)oxy)ethyl)-2-chloropyrazolo[1,5-a]pyrimidine-6-carboxylate (300 mg, 0.734 mmol) in MeOH (3 ml) was added 4N HCl in dioxane (3.67 ml, 14.67 mmol). The reaction mixture was stirred at RT overnight and concentrated to afford 7-((S)-1-(((R)-1-acetylpyrrolidin-3-yl)oxy)ethyl)-2-chloropyrazolo[1,5-a]pyrimidine-6-carboxylic acid. M/z=353-355 [M+H]+, Rt=0.60 min (UPLC Method B1).

### D13: 7-((S)-1-(((S)-1-acetylpyrrolidin-3-yl)oxy)ethyl)-2-chloropyrazolo[1,5-a]-pyrimidine-6-carboxylic acid

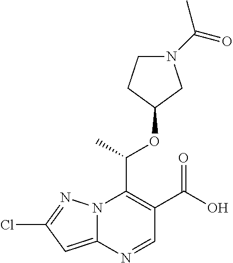 [SMILES: CC(=O)N1CC[C@H](O[C@@H](C)c2c(C(=O)O)cnc3cc(Cl)nn23)C1]

7-((S)-1-(((S)-1-acetylpyrrolidin-3-yl)oxy)ethyl)-2-chloropyrazolo[1,5-a]pyrimidine-6-carboxylic acid was prepared analogously as described for compound D12 using (S)-tert-butyl 3-(((S)-5-(tert-butoxy)-3,5-dioxopentan-2-yl)oxy)pyrrolidine-1-carboxylate instead of (R)-tert-butyl 3-(((S)-5-(tert-butoxy)-3,5-dioxopentan-2-yl)oxy)pyrrolidine-1-carboxylate in step a). M/z=353-355 [M+H]+, Rt=0.59 min (UPLC Method B1).

### D14: (S)-2-chloro-7-(1-methoxyethyl)pvrazolo[1,5-a]pyrimidine-6-carboxylic acid

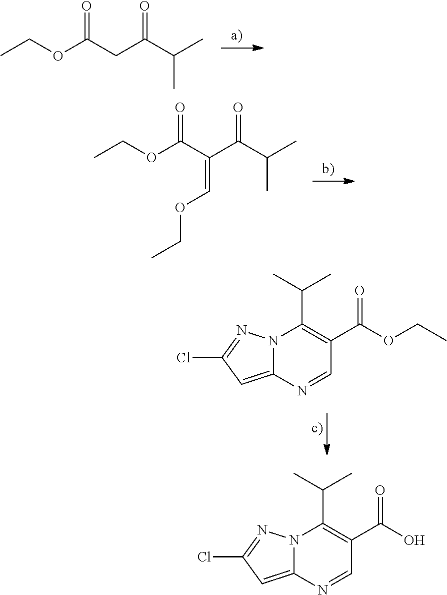

### a) ethyl 2-(ethoxymethylene)-4-methyl-3-oxopentanoate

Ethyl isobutyrylacetate (9.0 g, 56.9 mmol), triethyl orthoformate (18.9 ml, 114 mmol) and Ac₂O (10.7 ml, 114 mmol) were stirred at 135° C. overnight. The solution was concentrated (16 mbar/60° C.) to afford ethyl 2-(ethoxymethylene)-4-methyl-3-oxopentanoate as a cis/trans mixture M/z=215 [M+H]+, Rt=0.93 and 0.99 min (UPLC Method B2), ¹H NMR (400 MHz, DMSO-d₆) 7.84 and 7.66 (2s, 1H), 4.27-4.06 (m, 4H), 3.12-3.05 (m, 1H), 1.27-1.15 (m, 6H), 1.03-0.98 (m, 6H).

### b) (ethyl 2-chloro-7-isopropylpyrazolo[1,5-a]pyrimidine-6-carboxylate

Ethyl 2-(ethoxymethylene)-4-methyl-3-oxopentanoate (11.8 g, 55.1 mmol) and 5-chloro-1H-pyrazol-3-amine (6.15 g, 52.3 mmol) in EtOH (130 ml) were stirred at 80° C. overnight. Water was added to the reaction mixture and the aqueous phase was extracted with AcOEt. The organic phase was washed with aqueous saturated NaHCO₃, water and brine, dried over Na₂SO₄, filtered and concentrated. The crude product was purified by flash column chromatography on silica gel (cyclohexane/AcOEt: 1/0 to 9/1) to afford (ethyl 2-chloro-7-isopropylpyrazolo[1,5-a]pyrimidine-6-carboxylate. M/z=268-270 [M+H]+, Rt=1.27 min (UPLC Method B2), ¹H NMR (400 MHz, DMSO-d₆) δ 8.80 (s, 1H), 7.00 (s, 1H), 4.40-4.33 (m, 1H), 4.37 (q, 2H), 1.31 (d, 6H), 1.36 (t, 3H).

### c) 2-chloro-7-isopropylpyrazolo[1,5-a]pyrimidine-6-carboxylic acid

Ethyl 2-chloro-7-isopropylpyrazolo[1,5-a]pyrimidine-6-carboxylate (10.5 g, 39.3 mmol) was dissolved in EtOH (100 ml) and 2N NaOH (39.3 ml, 79 mmol) was added. The reaction mixture was stirred at 60° C. for 3 h. EtOH was evaporated, AcOEt was added and the mixture was acidified with 1M aqueous HCl to give a white suspension. The solid was filtered, washed with water and dried under vacuum. The resulting residue was treated with AcOEt and extracted with aqueous saturated NaHCO₃. The aqueous phase was separated, acidified to pH=2 and the precipitate filtered and washed with cold AcOEt to afford 2-chloro-7-isopropylpyrazolo[1,5-a]pyrimidine-6-carboxylic acid. M/z=240-242 [M+H]+, Rt=0.83 min (UPLC Method B2), ¹H NMR (400 MHz, DMSO-d₆) δ ppm: 8.83 (s, 1H), 6.98 (s, 1H), 4.58-4.47 (m, 1H), 1.51 (d, 6H).

### Part E: Synthesis of Amino-Pyridines

### E1: 6-(2H-1,2,3-triazol-2-yl)-5-(trifluoromethyl)pyridin-3-amine

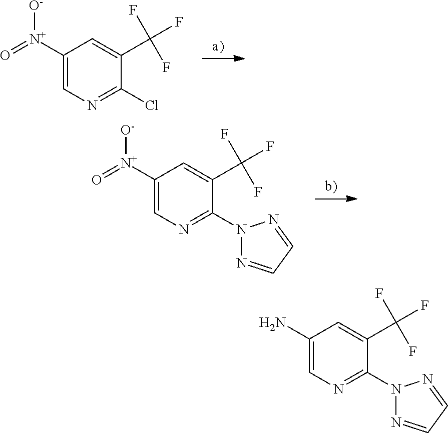

### a) 5-nitro-2-(2H-1,2,3-triazol-2-yl)-3-(trifluoromethyl)pyridine

To a solution of 2-chloro-5-nitro-3-(trifluoromethyl)pyridine (1.0 g, 4.41 mmol) and K₂CO₃ (1.22 g, 8.83 mmol) in THF (5 ml) was added 2H-1,2,3-triazole (0.31 ml, 5.30 mmol). The reaction mixture was stirred for 1h at RT. Water was added and the mixture was extracted with AcOEt. The organic layer was washed with brine, dried over Na₂SO₄, filtered and concentrated under vacuum. The residue was purified by flash column chromatography on silica gel (cyclohexane/AcOEt: 100/0 to 50/50) to afford 5-nitro-2-(2H-1,2,3-triazol-2-yl)-3-(trifluoromethyl)pyridine. M/z=260 [M+H]+, Rt=0.88 min (UPLC Method B1), ¹H NMR (400 MHz, DMSO-d₆) δ ppm: 9.69 (d, 1H), 9.17 (d, 1H), 8.37 (s, 2H).

### b) 6-(2H-1,2,3-triazol-2-yl)-5-(trifluoromethyl)pyridin-3-amine

To a solution of 5-nitro-2-(2H-1,2,3-triazol-2-yl)-3-(trifluoromethyl)pyridine (770 mg, 2.97 mmol) in 1.25M HCl in MeOH (48 ml, 59 mmol) at RT was added portionwise tin(II) chloride (2.82 g, 14.9 mmol). The reaction was stirred at RT for 2 h. 4N aq. NaOH was added and the solution was extracted with DCM. The organic layer was dried over anhydrous Na₂SO₄, filtered and concentrated under vacuum. The crude product was purified by flash column chromatography on silica gel (cyclohexane/AcOEt: 100/0 to 0/100) to afford 6-(2H-1,2,3-triazol-2-yl)-5-(trifluoromethyl)pyridin-3-amine. M/z=230 [M+H]+, Rt=0.64 min (UPLC Method B1), ¹H NMR (400 MHz, DMSO-d₆) δ ppm: 8.08 (d, 1H), 8.05 (s, 2H), 7.43 (d, 1H), 6.39 (s, 2H).

### E2: 5-chloro-6-(2H-1,2,3-triazol-2-yl)pyridin-3-amine

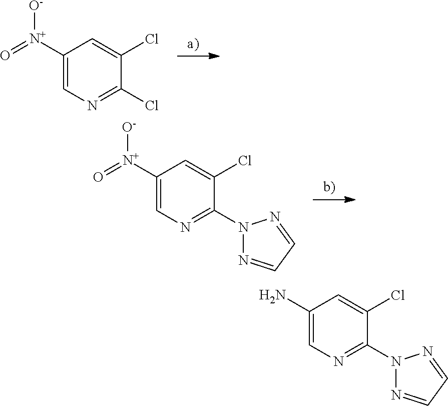

### a) 3-chloro-5-nitro-2-(2H-1,2,3-triazol-2-yl)pyridine

To a solution of 2,3-dichloro-5-nitropyridine (1.0 g, 5.18 mmol) and K₂CO₃ (1.43 g, 10.4 mmol) in THF (5 ml) was added 2H-1,2,3-triazole (0.360 ml, 6.22 mmol). The reaction mixture was stirred at RT overnight. Since the reaction was incomplete, additional 2H-1,2,3-triazole (0.300 ml, 5.18 mmol) was added and reaction mixture was stirred for additional 2 days at RT. Water was added and the mixture was extracted with AcOEt. The organic layer was washed with brine, dried over Na₂SO₄, filtered and concentrated under vacuum. The residue was taken-up in DCM, the solid was filtered off and the filtrate was concentrated. The residue was purified by flash column chromatography on silica gel (cyclohexane/AcOEt: 1/0 to 7/3) to afford 3-chloro-5-nitro-2-(2H-1,2,3-triazol-2-yl)pyridine. Rt=0.75 min (UPLC Method B1), ¹H NMR (400 MHz, DMSO-d₆) δ ppm: 9.39 (d, 1H), 9.15 (d, 1H), 8.33 (s, 2H).

### b) 5-chloro-6-(2H-1,2,3-triazol-2-yl)pyridin-3-amine

To a solution of 3-chloro-5-nitro-2-(2H-1,2,3-triazol-2-yl)pyridine (500 mg, 2.22 mmol) in 1.25M HCl in MeOH (35.5 ml, 44 mmol) at RT was added portionwise tin(II) chloride (2.1 g, 11.1 mmol). The reaction was stirred at RT for 2 h. The mixture was concentrated and the residue was diluted with DCM. The mixture was basified with 1N aq. NaOH and phases were separated. The organic layer was dried over anhydrous Na₂SO₄, filtered and concentrated under vacuum. The residue was purified by flash column chromatography on silica gel (cyclohexane/AcOEt: 1/0 to 0/1) to afford 5-chloro-6-(2H-1,2,3-triazol-2-yl)pyridin-3-amine. M/z=196-198 [M+H]+, Rt=0.50 min (UPLC Method B1), ¹H NMR (400 MHz, DMSO-d₆) δ ppm: 8.05 (s, 2H), 7.81 (s, 1H), 7.20 (s, 1H), 6.20 (d, 2H).

### E3: 5-amino-2-(2H-1,2,3-triazol-2-yl)nicotinonitrile

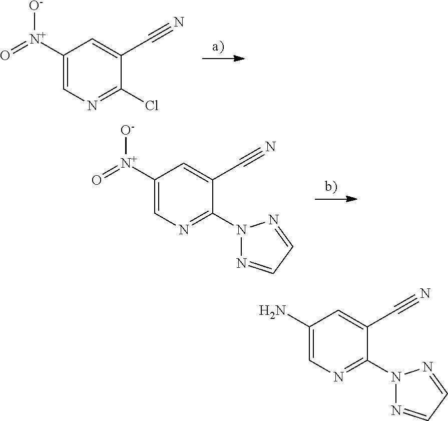

### a) 5-nitro-2-(2H-1,2,3-triazol-2-yl)nicotinonitrile

5-nitro-2-(2H-1,2,3-triazol-2-yl)nicotinonitrile was prepared analogously as described for compound E1 using 2-chloro-5-nitronicotinonitrile instead of 2-chloro-5-nitro-3-(trifluoromethyl)pyridine in step a). Rt=0.62 min (UPLC Method B2). ¹H NMR (400 MHz, DMSO-d₆) δ ppm: 9.61 (s, 1H), 9.46 (s, 1H), 8.47 (s, 2H).

### b) 5-amino-2-(2H-1,2,3-triazol-2-yl)nicotinonitrile

To a solution of 5-nitro-2-(2H-1,2,3-triazol-2-yl)nicotinonitrile (700 mg, 3.24 mmol) in EtOH (50 ml) was added Pd-C (10%) (517 mg, 0.486 mmol). The reaction vessel was equipped with a H₂-balloon, evacuated and purged with hydrogen. After 30 min stirring at RT, the reaction vessel was evacuated and purged with argon. The reaction mixture was filtered and rinsed thoroughly with EtOH. The filtrate was concentrated and dried in vacuo to afford 5-amino-2-(2H-1,2,3-triazol-2-yl)nicotinonitrile. M/z=187 [M+H]+, Rt=0.47 min (UPLC Method B2), ¹H NMR (400 MHz, DMSO-d₆) δ ppm: 8.15 (s, 2H), 8.11 (s, 1H), 7.47 (s, 1H), 6.29 (s, 2H).

### E4: 5-amino-2-methoxynicotinonitrile

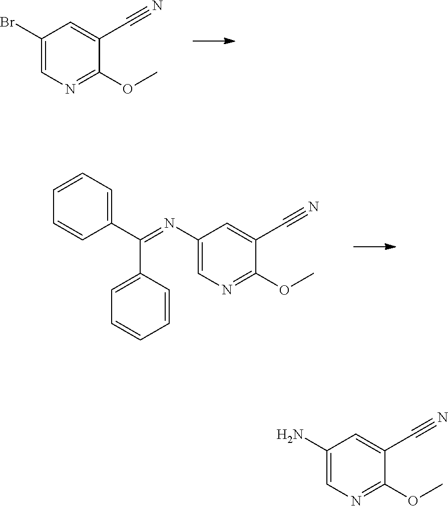 [SMILES: COc1ncc(Br)cc1C#N ; COc1ncc(N=C(c2ccccc2)c2ccccc2)cc1C#N ; COc1ncc(N)cc1C#N]

A Radley tube was charged with Pd(OAc)₂ (15.81 mg, 0.07 mmol) and xantphos (81 mg, 0.14 mmol) and purged with argon. 5-bromo-2-methoxynicotinonitrile (500 mg, 2.35 mmol), diphenylmethanimine (0.471 ml, 2.82 mmol), Cs₂CO₃ (1.53 g, 4.69 mmol) and dioxane (20 ml) were added and the mixture was heated for 15 h at 100° C. After cooling to RT, the reaction mixture was filtered, washed with ether and the filtrate was concentrated to give 5-((diphenylmethylene)amino)-2-methoxynicotinonitrile as an intermediate. This was dissolved in THF (20 ml), 2N aq. HCl (1.43 ml, 46.9 mmol) was added and the mixture was stirred for 10 min. The reaction mixture was diluted with water and cyclohexane/AcOEt (1:1; 50 ml). The phases were separated and the aqueous phase was extracted with AcOEt (2×30 ml). The combined organic layers were dried over Na₂SO₄, filtered and concentrated to give a mixture of crude product and benzophenone. The aqueous phase was neutralized by addition of 1N aq. NaOH and extracted with AcOEt (2×30 ml). The combined organic layers were dried over Na₂SO₄, filtered and concentrated. Both fractions were combined and purified by flash column chromatography on silica gel (cyclohexane/AcOEt: 4/1 to 0/1) to afford 5-amino-2-methoxynicotinonitrile. M/z=150 [M+H]+, Rt=0.60 min (UPLC Method B2), ¹H NMR (400 MHz, DMSO-d₆) δ ppm: 7.81 (d, 1H), 7.35 (d, 1H), 5.21 (s, 2H), 3.84 (s, 3H).

### E5: 2-(difluoromethyl)pyridin-4-amine

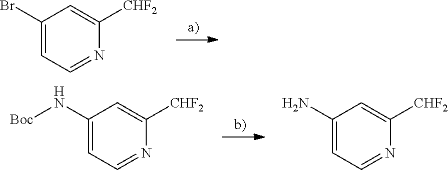

### a) tert-butyl (2-(difluoromethyl)pyridin-4-yl)carbamate

A mixture of 4-bromo-2-(difluoromethyl)pyridine (5.7 g, 27.4 mmol), tert-butyl carbamate (3.85 g, 32.9 mmol), xantphos (1.43 g, 2.47 mmol), tris(dibenzylideneacetone) dipalladium(0) (0.75 g, 0.82 mmol) and cesium carbonate (17.9 g, 54.8 mmol) in dioxane (120 ml) was stirred 90° C. for 72 h. The reaction mixture was filtered and concentrated. The crude product was purified by flash column chromatography on silica gel (cyclohexane/EtOAc: 100/0 to 50/50). M/z=245 [M+H]+, Rt=0.98 min (UPLC Method B1), ¹H NMR (400 MHz, DMSO-d₆) δ ppm: 10.1 (s, 1H), 8.42 (d, 1H), 7.81 (s, 1H), 7.50 (d, 1H), 6.85 (t, 1H), 1.50 (s, 9H).

### b) 2-(difluoromethyl)pyridin-4-amine

To a solution of tert-butyl (2-(difluoromethyl)pyridin-4-yl)carbamate (7.53 g, 27.4 mmol) in MeOH (25 ml) was added 4N HCl in dioxane (137 mL, 549 mmol). The solution was stirred at RT overnight. The reaction mixture was concentrated, quenched with sat aq NaHCO₃, extracted with EtOAc, dried over Na₂SO₄, filtered and concentrated. Purification by flash column chromatography on silica gel (cyclohexane/EtOAc: 100/0 to 0/100) yielded the title compound. M/z=145 [M+H]+, Rt=0.18 min (UPLC Method B1), ¹H NMR (400 MHz, DMSO-d₆) δ ppm: 8.03 (d, 1H), 6.75 (s, 1H), 6.66 (t, 1H), 6.56 (d, 1H), 6.35 (s, 2H).

### E6: 1-(4-amino-6-(trifluoromethyl)pyridin-2-yl)ethanol

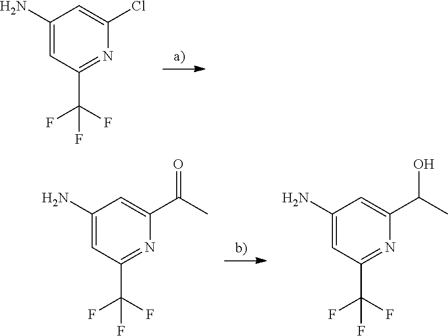

### a) 1-(4-amino-6-(trifluoromethyl)pyridin-2-yl)ethanone

A 30 ml ACE Glass (Sigma-Aldrich, 8648-03) was charged with 2-chloro-6-(trifluoromethyl)pyridin-4-amine (500 mg, 2.54 mmol), tributyl(1-ethoxyvinyl)stannane (1.09 ml, 3.05 mmol), PdCl₂(PPh₃)₂ (89 mg, 0.13 mmol) and cesium fluoride (850 mg, 5.60 mmol). The tube was purged with argon and dioxane (12.5 ml) was added. The vessel was sealed and the reaction mixture was stirred at 100° C. for 3 h. Tributyl(1-ethoxyvinyl)stannane (1.09 ml, 3.05 mmol) and PdCl₂(PPh₃)₂ (89 mg, 0.13 mmol) were again added and the reaction mixture was stirred at 100° C. during 5 h. The reaction mixture was concentrated, diluted with AcOEt and filtered through a plug of celite. The filtrate was concentrated to dryness, dissolved in THF (12.50 ml) and 1N aq. HCl (6.36 ml, 6.36 mmol) was added. The reaction mixture was stirred at RT overnight, concentrated and extracted with AcOEt. The organic layer was dried over a phase separator cartridge (1ST), concentrated and purified by flash column chromatography on silica gel (cyclohexane/AcOEt: 10/0 to 75/25) to afford 1-(4-amino-6-(trifluoromethyl)pyridin-2-yl)ethanone. M/z=205 [M+H]+, Rt=0.86 min (UPLC Method B2), ¹H NMR (400 MHz, DMSO-d₆) δ ppm: 7.25 (d, 1H), 7.08 (d, 1H), 6.90 (s, 2H), 2.54 (s, 3H).

### b) 1-(4-amino-6-(trifluoromethyl)pyridin-2-yl)ethanol

To a solution of 1-(4-amino-6-(trifluoromethyl)pyridin-2-yl)ethanone (430 mg, 1.20 mmol) in MeOH (6.15 ml) at 0° C., was added NaBH₄ (47.7 mg, 1.26 mmol). The reaction was stirred at 0° C. for 1 h, poured into water and extracted three times with AcOEt. The combined organic layers were washed with brine and water, dried over a phase separator cartridge (1ST), concentrated and purified by flash column chromatography on silica gel (cyclohexane/AcOEt: 10/0 to 8/2) to afford 1-(4-amino-6-(trifluoromethyl)pyridin-2-yl)ethanol. M/z=207 [M+H]+, Rt=0.58 min (UPLC Method B2), ¹H NMR (400 MHz, DMSO-d₆) δ ppm: 6.84 (d, 1H), 6.74 (d, 1H), 6.51 (s, 2H), 5.28 (d, 1H), 4.54 (qd, 1H), 1.29 (d, 3H).

### E7: 1-(4-aminopyridin-2-yl)-2,2,2-trifluoroethanol

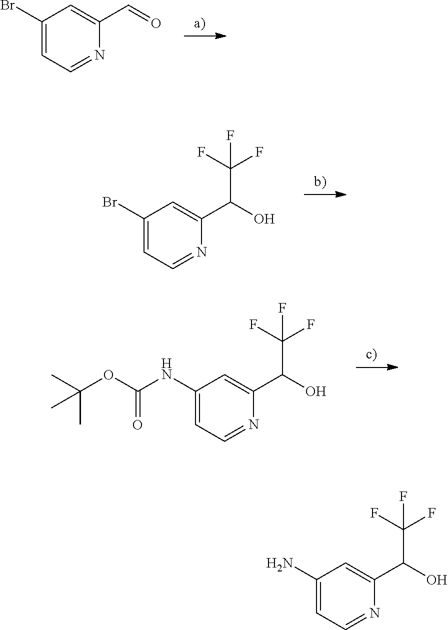 [SMILES: O=Cc1cc(Br)ccn1 ; OC(c1cc(Br)ccn1)C(F)(F)F ; CC(C)(C)OC(=O)Nc1ccnc(C(O)C(F)(F)F)c1 ; Nc1ccnc(C(O)C(F)(F)F)c1]

### a) 1-(4-bromopyridin-2-yl)-2,2,2-trifluoroethanol

To a solution of 4-bromopicolinaldehyde (1 g, 5.38 mmol) and trimethyl(trifluoromethyl)-silane (0.92 g, 6.45 mmol) in THF (10 ml) at 0° C., under inert atmosphere, was added TBAF 1M in THF (0.27 mL, 0.27 mmol). After 30 min at 0° C., the reaction was allowed to warm to RT for 2 h. To the reaction mixture was added 1N aq. HCl (6 ml) and the solution was stirred for 30 min at RT. Then, 1N aq. NaOH was added to pH 8 and the mixture was extracted with AcOEt. The organic layer was dried over Na₂SO₄, filtered, concentrated and purified by flash column chromatography on silica gel (cyclohexane/AcOEt: 10/0 to 5/5) to afford 1-(4-bromopyridin-2-yl)-2,2,2-trifluoroethanol. M/z=256-258 [M+H]+, Rt=0.85 min (UPLC Method B1), ¹H NMR (400 MHz, DMSO-d₆) δ ppm: 8.49 (d, 1H), 7.81 (d, 1H), 7.77-7.70 (m, 1H), 7.19 (s, 1H), 5.17 (m, 1H).

### b) tert-butyl (2-(2,2,2-trifluoro-1-hydroxyethyl)pyridin-4-yl)carbamate

A mixture of 1-(4-bromopyridin-2-yl)-2,2,2-trifluoroethanol (1.18 g, 4.61 mmol), tert-butyl carbamate (0.65 g, 5.53 mmol), xantphos (0.24 g, 0.42 mmol), tris(dibenzylideneacetone)dipalladium(0) (0.13 g, 0.14 mmol) and cesium carbonate (3.00 g, 9.22 mmol) in dioxane (20 ml) under inert atmosphere was stirred overnight at 90° C. After cooling to RT, the reaction mixture was filtered, concentrated and purified by flash column chromatography on silica gel (cyclohexane/AcOEt: 10/0 to 7/3) to afford tert-butyl (2-(2,2,2-trifluoro-1-hydroxyethyl)pyridin-4-yl)carbamate. M/z=293 [M+H]+, Rt=0.90 min (UPLC Method B1).

### c) 1-(4-aminopyridin-2-yl)-2,2,2-trifluoroethanol

To a solution of tert-butyl (2-(2,2,2-trifluoro-1-hydroxyethyl)pyridin-4-yl)carbamate (610 mg, 2.09 mmol) in MeOH (2 ml) was added 4N HCl in dioxane (10.44 ml, 41.7 mmol). The reaction mixture was stirred at RT overnight, concentrated, partitioned between sat. aq. NaHCO₃ and AcOEt. The organic layer was dried over Na₂SO₄, filtered, concentrated and purified by flash column chromatography on silica gel (cyclohexane/AcOEt: 10/0 to 0/10) to afford 1-(4-aminopyridin-2-yl)-2,2,2-trifluoroethanol. ¹H NMR (400 MHz, DMSO-d₆) δ ppm: 7.94 (m, 1H), 6.72 (d, 1H), 6.63 (d, 1H), 6.45 (m, 1H), 6.16 (s, 2H), 4.96-4.77 (m, 1H).

### E8: 5-chloro-2-(2-methoxyethoxy)pyridin-3-amine

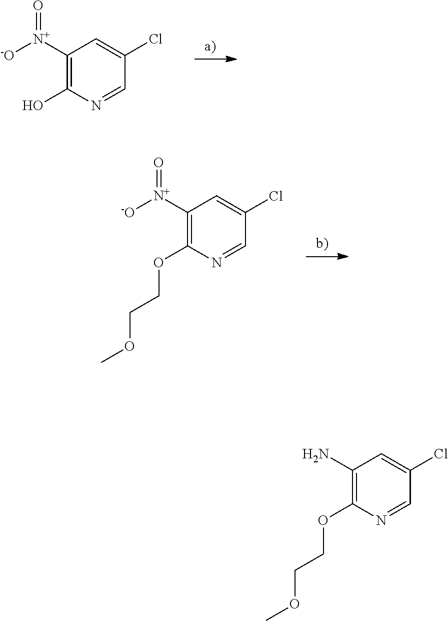

### a) 5-chloro-2-(2-methoxyethoxy)-3-nitropyridine

To a solution of 5-chloro-2-hydroxy-3-nitropyridine (1.23 g, 6.92 mmol), 3-methoxypropan-1-ol (0.71 ml, 7.62 mmol) and PPh₃ (2.04 g, 7.62 mmol) in THF (10 ml) was added DEAD (1.24 ml, 7.62 mmol) dropwise at 0° C. The reaction mixture was stirred at RT overnight. The reaction mixture was concentrated and purified by flash column chromatography on silica gel (heptane/AcOEt: 10/0 to 0/10) to afford 5-chloro-2-(2-methoxyethoxy)-3-nitropyridine. M/z=233-235 [M+H]+, Rt=0.95 (UPLC Method B2), ¹H NMR (600 MHz, DMSO-d₆) δ ppm: 8.63 (d, 1H), 8.58 (d, 1H), 4.56 (m, 2H), 3.69 (m, 2H), 3.32 (s, 3H).

### b) 5-chloro-2-(2-methoxyethoxy)pyridin-3-amine

To a solution of 5-chloro-2-(2-methoxyethoxy)-3-nitropyridine (740 mg, 3.18 mmol) in acetic acid (15 ml) was added iron powder (1.78 g, 31.8 mmol) and the reaction mixture was stirred at RT for 2.5 h. The reaction mixture was concentrated, DCM (50 ml) was added and the mixture was stirred for 10 min. The mixture was filtered and the filtrate was washed with sat. aq. NaHCO₃, water and brine. The organic phase was dried over Na₂SO₄, filtered and the solvent was evaporated to afford 5-chloro-2-(2-methoxyethoxy)pyridin-3-amine. M/z=203-205 [M+H]+, Rt=0.78 (UPLC Method B2), ¹H NMR (400 MHz, DMSO-d₆) δ ppm: 7.31 (d, 1H), 6.90 (d, 1H), 5.25 (s, 2H), 4.36 (t, 2H), 3.67 (t, 2H), 3.30 (s, 3H).

### E9: 4-amino-6-chloro-N,N-dimethylpicolinamide

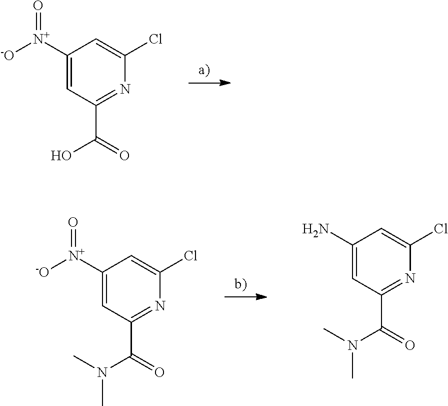

### a) 6-chloro-N,N-dimethyl-4-nitropicolinamide

To a solution of 6-chloro-4-nitro-2-pyridinecarboxylic acid (500 mg, 2.47 mmol) in DMF (10 ml) was added HOBt (454 mg, 2.96 mmol) and the reaction mixture was stirred at RT for 1 h. Dimethylamine hydrochloride (200 mg, 2.47 mmol) and EDC hydrochloride (568 mg, 2.96 mmol) were added and the reaction mixture was stirred at RT for 3 days. The crude mixture was poured into sat. aq. NaHCO₃ and extracted three times with AcOEt. The combined organic layers were washed with water, brine, dried over Na₂SO₄, filtered and concentrated to afford 6-chloro-N,N-dimethyl-4-nitropicolinamide which was used in the next step without further purification. M/z=230-232 [M+H]+, Rt=0.73 (UPLC Method B2).

### b) 4-amino-6-chloro-N,N-dimethylpicolinamide

To a solution of 6-chloro-N,N-dimethyl-4-nitropicolinamide (380 mg, 1.66 mmol) in AcOH (10 ml) was added iron (924 mg, 16.6 mmol) and the reaction mixture was stirred at RT for 1.5 h. The reaction mixture was concentrated, DCM (50 ml) was added and the mixture was stirred for 10 min. The mixture was filtered and the filtrate was washed with sat.aq. NaHCO₃, water and brine. The organic phase was dried over Na₂SO₄, filtered and the solvent was evaporated to afford 4-amino-6-chloro-N,N-dimethylpicolinamide. M/z=200-202 [M+H]+, Rt=0.48 (UPLC Method B2).

### E10: 5-amino-3-chloro-N,N-dimethylpicolinamide

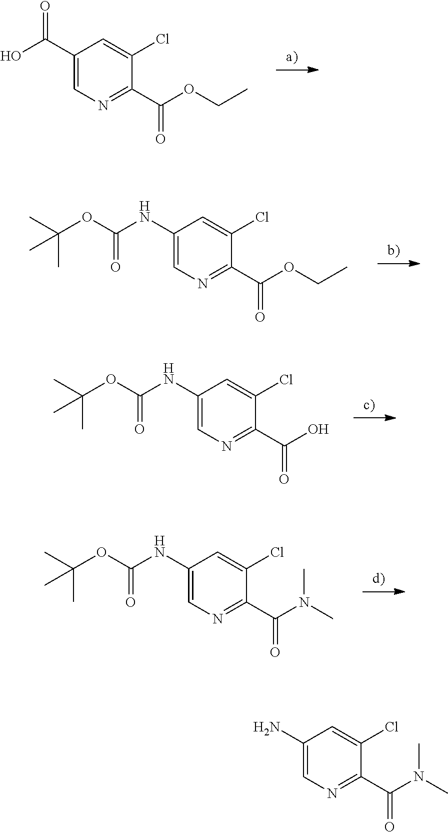

### a) ethyl 5-((tert-butoxycarbonyl)amino)-3-chloropicolinate

To a solution of 5-chloro-6-(ethoxycarbonyl)nicotinic acid (920 mg, 4.01 mmol) in dry dioxane, under inert atmosphere, were added DPPA (951 ml, 4.21 mmol) and TEA (2.78 ml, 20.0 mmol). The reaction mixture was stirred at RT for 30 min then a solution of 2-methylpropan-2-ol (7.7 ml, 80 mmol) in dry dioxane (10 ml) was added. The reaction mixture was stirred at 80° C. for 2 h. The reaction mixture was poured in brine and extracted three times with AcOEt. The combined organic layers were dried over Na₂SO₄, filtered, concentrated and purified by flash column chromatography on silica gel (heptane/AcOEt: 9/1 to 0/10) to afford ethyl 5-((tert-butoxycarbonyl)amino)-3-chloropicolinate. M/z=301-303 [M+H]+, Rt=1.10 (UPLC Method B2).

### b) 5-((tert-butoxycarbonyl)amino)-3-chloropicolinic acid

To a solution of ethyl 5-((tert-butoxycarbonyl)amino)-3-chloropicolinate (562 mg, 1.87 mmol) in EtOH (5 ml) was added 2N aq. NaOH (2.80 ml, 5.61 mmol) and the reaction mixture was stirred at RT for 2 h. The mixture was concentrated, poured into water, acidified with 2N aq. HCl and extracted with AcOEt. The organic layer was washed with water, brine, dried over Na₂SO₄, filtered and concentrated to afford 5-((tert-butoxycarbonyl)amino)-3-chloropicolinic acid. M/z=273-275 [M+H]+, Rt=0.77 (UPLC Method B2).

### c) tert-butyl (5-chloro-6-(dimethylcarbamoyl)pyridin-3-yl)carbamate

To a solution of 5-((tert-butoxycarbonyl)amino)-3-chloropicolinic acid (170 mg, 0.62 mmol) in DMF (5 ml) was added HOBt (143 mg, 0.93 mmol) and the reaction mixture was stirred at RT for 1 h. Then dimethylamine 2M in THF (0.94 ml, 1.87 mmol) and EDC hydrochloride (179 mg, 0.93 mmol) were added and the reaction mixture was stirred at RT for 4 h. To complete the reaction, dimethylamine 2M in THF (0.94 ml, 1.87 mmol) was added again and the reaction mixture was stirred at RT overnight. The crude reaction mixture was poured into sat aq. NaHCO₃ and extracted three times with AcOEt. The combined organic layers were washed with water, brine, dried over Na₂SO₄, filtered, concentrated and purified by flash column chromatography on silica gel (heptane/AcOEt: 8/2 to 0/10) to afford tert-butyl (5-chloro-6-(dimethylcarbamoyl)-pyridin-3-yl)carbamate. M/z=300-302 [M+H]+, Rt=0.88 (UPLC Method B2).

### d) 5-amino-3-chloro-N,N-dimethylpicolinamide

To tert-butyl (5-chloro-6-(dimethylcarbamoyl)pyridin-3-yl)carbamate (65 mg, 0.217 mmol) was added 4N HCl in dioxane (0.1 ml, 0.434 mmol) and the reaction mixture was stirred at RT overnight. To complete the reaction, TFA (2 ml) was added and the reaction mixture was stirred at RT overnight. The reaction mixture was concentrated to afford 5-amino-3-chloro-N,N-dimethylpicolinamide as the corresponding TFA-salt. M/z=200-202 [M+H]+, Rt=0.45 (UPLC Method B2).

### E11: (5-amino-3-chloropyridin-2-yl)(pyrrolidin-1-yl)methanone

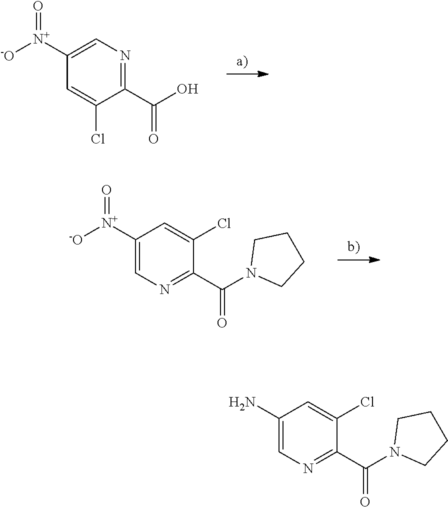

### a) (3-chloro-5-nitropyridin-2-yl)(pyrrolidin-1-yl)methanone

To a solution of 3-chloro-5-nitropicolinic acid (250 mg, 1.23 mmol) in DMF (5 ml) was added HOBt (284 mg, 1.85 mmol), followed by pyrrolidine (132 mg, 1.85 mmol) and EDC (355 mg, 1.85 mmol). The reaction mixture was stirred at RT for 16 h. Saturated NaHCO3 solution was added, the mixture was extracted 3 times with AcOEt and the organic phase was washed with water and brine. The organic phase was dried and the solvent evaporated to yield the title compound which was used in the following step without further purification. M/z=256-258 [M+H]+, Rt=0.74 min (UPLC Method B1)

### b) (5-amino-3-chloropyridin-2-yl)(pyrrolidin-1-yl)methanone

To a solution of (3-chloro-5-nitropyridin-2-yl)(pyrrolidin-1-yl)methanone (218 mg, 0.85 mmol) in EtOH (10 ml) and aqueous sat. NH4Cl solution (5 ml) was added iron powder (473 mg, 8.53 mmol). The mixture was stirred at reflux for 2.5 h. After cooling to RT the reaction mixture was treated with AcOEt/water, 2:1 (100 ml) and the mixture was filtered over Celite. The organic phase was washed with water and brine, dried over Na₂SO₄ and the solvent was evaporated. Flash column chromatography (heptane, AcOEt: 100/0 to 0/100, followed by elution with MeOH) provided the title compound. M/z=226-228 [M+H]+, Rt=0.55 min (UPLC Method B1).

### E12: 5-amino-3-chloro-N-methylpicolinamide

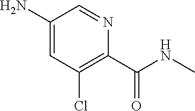

5-amino-3-chloro-N-methylpicolinamide was prepared as described for compound E10 using 5-chloro-6-(ethoxycarbonyl)nicotinic acid and methylamine instead of dimethylamine in step a). M/z=186-188 [M+H]+, Rt=0.39 min (UPLC Method B1).

### E13: (S)-tert-butyl (1-((3-amino-5-chloropyridin-2-yl)oxy)propan-2-yl)carbamate

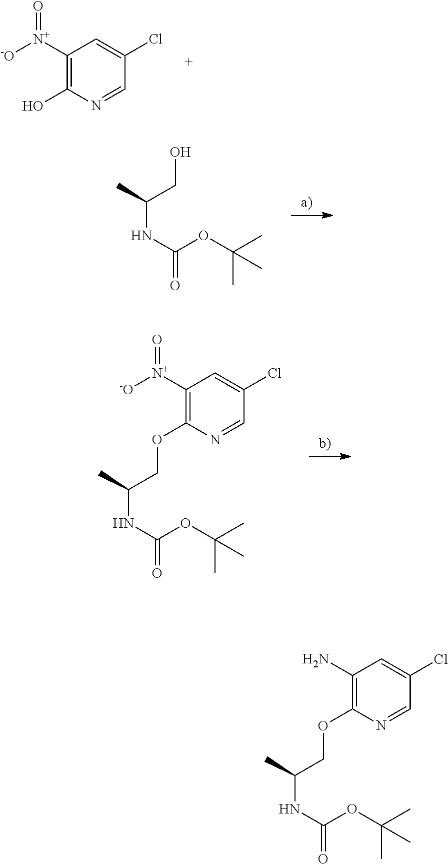

### a) (S)-tert-butyl (1-((5-chloro-3-nitropyridin-2-yl)oxy)propan-2-yl)carbamate

To a suspension of 5-chloro-3-nitropyridin-2-ol (1.02 g, 5.86 mmol), N-Boc-L-alaninol (1.14 g, 6.45 mmol) and PPh₃ (1.72 g, 6.45 mmol) in THF (10 ml) at 0° C., was added dropwise DEAD (1.02 ml, 6.45 mmol) over a period of 5 min. The reaction mixture was stirred at RT overnight. To complete the reaction, N-Boc-L-alaninol (550 mg), PPh₃ (850 mg) and DEAD (0.5 ml) were added and the reaction mixture was stirred for 3h at RT, poured into water and extracted three times with AcOEt. The combined organic layers were washed with sat. aq. NaHCO₃ and brine, dried over Na₂SO₄, filtered, concentrated and purified by flash column chromatography on silica gel (heptane/AcOEt: 10/0 to 3/7) to afford (S)-tert-butyl (1-((5-chloro-3-nitropyridin-2-yl)oxy)propan-2-yl)carbamate. M/z=332-334 [M+H]+, Rt=1.17 min (UPLC Method B2), ¹H NMR (400 MHz, DMSO-d₆) δ ppm: 8.63 (s, 1H), 8.59 (s, 1H), 6.86 (d, 1H), 4.42 (dd, 1H), 4.22 (dd, 1H), 3.89 (m, 1H), 1.34 (s, 9H), 1.11 (d, 3H).

### b) (S)-tert-butyl (1-((3-amino-5-chloropyridin-2-yl)oxy)propan-2-yl)carbamate

To a suspension of (S)-tert-butyl (1-((5-chloro-3-nitropyridin-2-yl)oxy)propan-2-yl)carbamate (1.09 g, 3.29 mmol) in EtOH (15 ml) and sat. aq. NH₄Cl (5 ml) was added iron powder (1.84 g, 32.9 mmol). The suspension was stirred at 80° C. for 2 h, concentrated and purified by flash column chromatography on silica gel (DCM/MeOH+1%NH₃: 10/0 to 7/3) to afford (S)-tert-butyl (1-((3-amino-5-chloropyridin-2-yl)oxy)propan-2-yl)carbamate. M/z=302-304 [M+H]+, Rt=1.07 min (UPLC Method B2), ¹H NMR (400 MHz, DMSO-d₆) δ ppm: 7.28 (d, 1H), 6.95 (d, 1H), 6.86 (d, 1H), 5.39 (s, 2H), 4.13 (dd, 1H), 3.99 (dd, 1H), 3.95-3.85 (m, 1H),1.39 (s, 9H), 1.12 (d, 3H).

General Comments re Final Products:

As shown above in the description for the general synthesis route, the compounds listed below were obtained by reacting the pyrazolo[1,5-a]pyrimidine-6-carboxylic acids as described in Section D1-D14 with the appropriate amino-pyrdine derivatives as described in Section E1-E13 in a Curtius Rearrangement Reaction via the corresponding isocyanates as intermediates to yield the urea derivatives shown below.

### EXAMPLE 1

### (S)-1-(5-cyanopyridin-3-yl)-3-(7-(1-methoxyethyl)-2-methylpyrazolo [1,5-a]pyrimidin-6-yl)urea

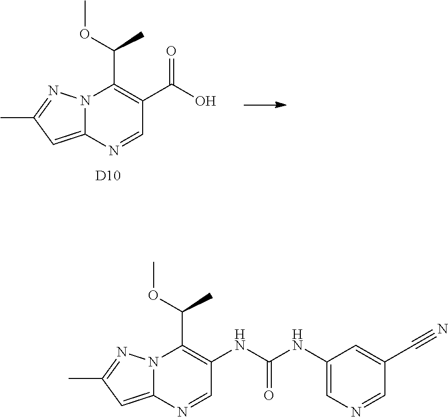

To a solution of (S)-7-(1-methoxyethyl)-2-methylpyrazolo[1,5-a]pyrimidine-6-carboxylic acid (100 mg, 0.43 mmol) in dioxane (2.8 ml) were added DPPA (113 μl, 0.51 mmol) and Et₃N (178 μl, 1.28 mmol). The reaction mixture was stirred at RT for 30 min. Then, 5-aminonicotinonitrile (152 mg, 1.28 mmol) was added and the reaction mixture was stirred at 100° C. for 10 min. The mixture was cooled to RT, evaporated and the residue was dissolved in AcOEt. The organic layers was washed with aq. sat. NaHCO₃ and dried over a phase separator cartridge (IST), concentrated and purified by flash column chromatography on silica gel (cyclohexane/AcOEt: 10/0 to 0/10) to afford (S)-1-(5-cyanopyridin-3-yl)-3-(7-(1-methoxyethyl)-2-methylpyrazolo [1,5-a]pyrimidin-6-yl)urea. M/z=352 [M+H]+, Rt=2.87 min (HPLC Method C1), ¹H NMR (400 MHz, DMSO-d₆) δ ppm: 10.02 (s, 1H), 8.83 (t, 1H), 8.74 (d, 1H), 8.63 (d, 1H), 8.42 (s, 1H), 8.39 (s, 1H), 6.56 (s, 1H), 5.50 (q, 1H), 3.33 (s, 3H), 2.44 (s, 3H), 1.57 (d, 3H).

To remove any material which epimerized during the course of the synthesis the compound can be purified to >98% enantiomeric excess by preparative chiral SFC: Instrument: Thar SuperPure200, column: Chiralpak IA 5 μM, 250×30 mm, 40° C., mobile phase: CO2/EtOH, 60/40.

Crystalline material was obtained by disolving the compound in acetonitrile (10 ml/g) at reflux. The heating was switched off and the solution was allowed to cool down slowly to 23° C. overnight under stirring. The resulting solid was collected by filtration and washed with small amounts of acetonitrile and dried over night under high vacuum at 50° C.

Powder X-Ray Diffraction Peaks of Example 1

| Angle | d Value | Rel. Intensity |
|---|---|---|
| °2θ | Å | % |
| 11.36 | 7.79 | 71.10 |
| 12.09 | 7.31 | 12.60 |
| 13.06 | 6.77 | 31.70 |
| 13.13 | 6.74 | 33.40 |
| 14.80 | 5.98 | 32.30 |
| 15.19 | 5.83 | 6.90 |
| 19.58 | 4.53 | 33.00 |
| 20.75 | 4.28 | 12.90 |
| 21.11 | 4.20 | 8.70 |
| 24.47 | 3.63 | 7.60 |
| 25.49 | 3.49 | 100.00 |
| 26.78 | 3.33 | 16.00 |
| 27.23 | 3.27 | 23.90 |
| 28.75 | 3.10 | 22.70 |

Solubility:

| Media | Solubility (mg/mL) [pH] |
|---|---|
| HCl/KCl pH 1.0 | 0.049 [1.10] |
| Simulated Gastric Fluid (SGF) pH 2.0 | 0.024 [2.00] |
| 100 mM Sodium Acetate pH 4.0 | 0.019 [4.04] |
| 19.12 mM Sodium Maleate Fasted state | 0.018 [6.63] |
| simulated intestinal fluid (FaSSIF) pH 6.5 | |
| FaSSIF pH 6.5 | 0.022 [6.56] |
| Fed state intestinal fluid FeSSIF pH 5.8 | 0.027 [5.85] |
| Water | 0.019 [7.65] |

### EXAMPLE 2

### (S)-1-(2-(difluoromethyl)pyridin-4-yl)-3-(2-fluoro-7-(1-methoxyethyl) pyrazolo[1,5-a]pyrimidin-6-yl)urea

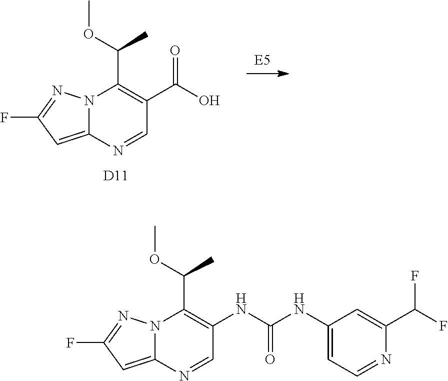

To a solution of 2-(difluoromethyl)isonicotinic acid (48.4 mg, 0.280 mmol) in dioxane (1 ml) were added DPPA (90 mg, 0.326 mmol) and Et₃N (0.1 ml, 0.699 mmol and the reaction mixture was stirred at RT for 1h. Then, (S)-2-fluoro-7-(1-methoxyethyl)pyrazolo[1,5-a]pyrimidine-6-carboxylic acid (70 mg, 0.233 mmol) was added and the reaction mixture was stirred at 100° C. for 1 h. The mixture was cooled to RT, poured into brine and extracted with AcOEt. The combined organic layers were dried dried over a phase separator cartridge (IST), concentrated and purified by flash column chromatography on silica gel (cyclohexane/AcOEt: 10/0 to 5/5) to afford (S)-1-(2-(difluoromethyl)pyridin-4-yl)-3-(2-fluoro-7-(1-methoxyethyl) pyrazolo[1,5-a]pyrimidin-6-yl)urea. M/z=381 [M+H]+, Rt=2.74 min (HPLC Method C1), ¹H NMR (400 MHz, DMSO-d₆) δ ppm: 10.19 (bs, 1H), 8.88 (s, 1H), 8.47 (m, 2H), 7.87 (d, 1H), 7.54-7.50 (m, 1H), 6.89 (t, 1H), 6.56 (d, 1H), 5.33 (q, 1H), 3.30 (s, 3H), 1.56 (d, 3H).

To remove any material which epimerized during the course of the synthesis the compound can be purified to >98% enantiomeric excess by preparative chiral SFC: Instrument: Thar SuperPure200, column: Chiralpak AD-H 5 μM, 250×30 mm, 40° C., mobile phase: CO2/EtOH, 85/15.

Crystalline hydrated form was obtained for compound of example No 2 and the process of making the form is described.

To 100 mg of (S)-1-(2-(difluoromethyl)pyridin-4-yl)-3-(2-fluoro-7-(1-methoxyethyl) pyrazolo[1,5-a]pyrimidin-6-yl)urea, 1 ml of methanol/water in a ratio of 4:1 (volume/volume) was added to form a suspension at room temperature. This suspension was stirred at ambient condition for 12 hrs. The resulting solid was collected by vacuum filtration and dried at 40° C. under vacuum for 12 hours.

Solubility Comparison of Example No 2

| mg/mL [pH] | Free form | | hydrate | |
|---|---|---|---|---|
| HCl/KCl pH 1.0 | >2.0 | [1.12] | >2.0 | [1.14] |
| Simulated Gastric Fluid (SGF) | 0.338 | [1.99] | 0.286 | [2.55] |
| pH 2.0 | | | | |
| 100 mM Sodium Acetate pH 4.0 | 0.032 | [3.99] | 0.030 | [4.03] |
| 19.12 mM Sodium Maleate | 0.026 | [6.61] | 0.025 | [6.54] |
| (blank FaSSIF) pH 6.5 | | | | |
| Fasted state simulated | 0.041 | [6.64] | 0.032 | [6.67] |
| intestinal fluid FaSSIF (V2) | | | | |
| pH 6.5 | | | | |
| Fed state intestinal fluid | 0.093 | [5.85] | 0.083 | [5.84] |
| FeSSIF (V2) pH 5.8 | | | | |
| Water | 0.029 | [7.76] | 0.026 | [6.67] |

Powder X-Ray Diffraction Peaks of Example No. 2 Hydrate

| Angle | d value | Intensity |
|---|---|---|
| °2θ | Å | % |
| 8.58 | 10.29 | 54.00 |
| 11.21 | 7.89 | 53.50 |
| 12.29 | 7.20 | 37.60 |
| 14.25 | 6.21 | 33.90 |
| 14.54 | 6.09 | 38.10 |
| 14.95 | 5.92 | 45.10 |
| 16.51 | 5.36 | 31.00 |
| 17.89 | 4.95 | 35.50 |
| 19.11 | 4.64 | 39.90 |
| 19.67 | 4.51 | 52.40 |
| 22.01 | 4.03 | 100.00 |
| 22.99 | 3.87 | 47.80 |
| 24.19 | 3.68 | 45.30 |
| 24.94 | 3.57 | 34.60 |
| 26.06 | 3.42 | 40.50 |

### EXAMPLE 3

### (S)-1-(2-chloro-7-(1-methoxyethyl)pyrazolo[1,5-a]pyrimidin-6-yl)-3-(2-(trifluoromethyl)pyridin-4-yl)urea

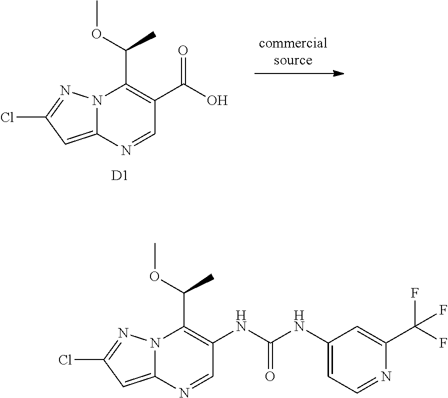

To a solution of (S)-2-chloro-7-(1-methoxyethyl)pyrazolo[1,5-a]pyrimidine-6-carboxylic acid (1.3 g, 4.78 mmol) (obtained in D1) in 1,4-dioxane (10 ml) were added DPPA (1.24 ml, 5.74 mmol) and Et₃N (3.33 ml, 23.9 mmol). The reaction mixture was stirred at RT for 30 min. Then, 2-(trifluoromethyl)pyridin-4-amine (1.55 g, 9.56 mmol) was added and reaction mixture was stirred at 100° C. for 2 h. The mixture was partitioned between AcOEt and saturated aqueous NaHCO₃ and the phases were separated. The organic layer was dried over Na₂SO₄, filtered and concentrated. The crude material was purified by flash column chromatography on silica gel (cyclohexane/AcOEt: 1/0 to 0/1). The residue was then taken up in MeOH and heated until disolution. After cooling to RT the precipitate was collected by filtration, washed with MeOH and dried to afford (S)-1-(2-chloro-7-(1-methoxyethyl)pyrazolo[1,5-a]pyrimidin-6-yl)-3-(2-(trifluoromethyl)pyridin-4-yl)urea. M/z=415-417 [M+H]+, Rt=4.18 min (HPLC Method C1), ¹H NMR (400 MHz, DMSO-d₆) δ ppm: 10.38 (s, 1H), 8.92 (s, 1H), 8.57 (s, 1H), 8.56 (s, 1H), 8.06 (d, 1H), 7.61 (dd, 1H), 6.94 (s, 1H), 5.41 (q, 1H), 3.32 (s, 3H), 1.57 (d, 3H).

Crystalline hydrated form was obtained for compound of example No 3: (S)-1-(2-chloro-7-(1-methoxyethyl)pyrazolo[1,5-a]pyrimidin-6-yl)-3-(2-(trifluoromethyl)pyridin-4-yl)urea was dissolved in acetonitrile (5 ml/g) and then heated to 50-60° C. Water (5 ml/g) was added to the solution in 1-2 hrs while the inner temperature was kept between 50-60° C. The mixture was cooled to RT in 1 h and kept at that temperature for 1 additional hour. After filtration the cake was dried at 55-60° C. under vaccum.

Powder X-Ray Diffraction Peaks of Example 3 Hemi-Hydrate

| Angle | d Value | Rel. Intensity |
|---|---|---|
| °2θ | Å | % |
| 12.94 | 6.84 | 11.20 |
| 13.51 | 6.55 | 15.10 |
| 15.03 | 5.89 | 49.40 |
| 15.48 | 5.72 | 14.30 |
| 16.59 | 5.34 | 21.40 |
| 19.93 | 4.45 | 100.00 |
| 21.31 | 4.17 | 23.40 |
| 22.00 | 4.04 | 13.20 |
| 22.96 | 3.87 | 24.10 |
| 24.22 | 3.67 | 36.10 |
| 24.62 | 3.61 | 20.20 |
| 26.53 | 3.36 | 22.40 |
| 27.24 | 3.27 | 10.50 |
| 28.49 | 3.13 | 20.20 |

Solubility of Example 3 Hemi-Hydrate:

| Media | Solubility (mg/mL) [pH] | |
|---|---|---|
| Water | 0.003 | [7.68] |
| pH 1.0 | 0.0016 | [1.14] |
| pH 4.0 | 0.0032 | [3.93] |
| pH 7.0 | 0.0026 | [6.96] |
| pH 9.0 | 0.003 | [8.82] |
| Simulated Gastric Fluid | 0.0222 | [2.05] |
| (SGF) | | |
| Fasted state simulated | 0.0047 | [6.55] |
| intestinal fluid (FaSSIF) | | |
| Fed state intestinal fluid | 0.0422 | [5.56] |
| (FeSSIF) | | |

### EXAMPLE 4

### 1-(5-chloro-6-(2H-1,2,3-triazol-2-yl)pyridin-3-yl)-3-(2-chloro-7-isopropylpyrazolo[1,5-a]pyrimidin-6-yl)urea

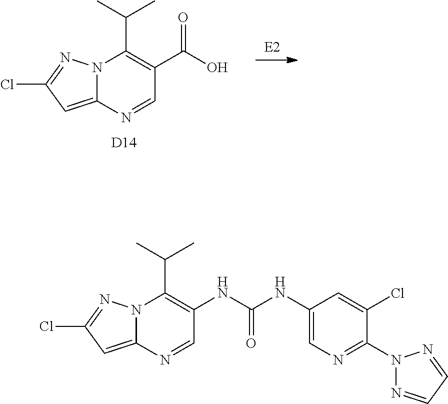

To a solution of 2-chloro-7-isopropylpyrazolo[1,5-a]pyrimidine-6-carboxylic acid (1.6 g, 6.01 mmol) in 1,4-dioxane (9 ml) were added DPPA (1.42 ml, 6.61 mmol) and Et₃N (3.35 ml, 24.0 mmol). The reaction mixture was stirred at RT for 30 min. Then, 5-chloro-6-(2H-1,2,3-triazol-2-yl)pyridin-3-amine (1.65 g, 8.41 mmol) was added and the reaction mixture was stirred at 100° C. for 2 h. The mixture was allowed to cool to RT, diluted with brine and extracted with AcOEt. The combined organic layers were dried over a phases separator cartouche and concentrated. The crude material was purified by flash column chromatography on silica gel (DCM/MeOH: 1/0 to 9/1), followed by prep. HPLC purification (method A). The combined fractions were washed with NaHCO3 solution, the organic phase dried and concentrated to a volume of 80 ml. The solution was cooled in an ice bath for 3 h and the precipitate was collected by filtration and dried to afford 1-(5-chloro-6-(2H-1,2,3-triazol-2-yl)pyridin-3-yl)-3-(2-chloro-7-isopropylpyrazolo[1,5-a]pyrimidin-6-yl)urea. M/z=432-434 [M+H]+, Rt=4.31 min (HPLC Method C1), ¹H NMR (400 MHz, DMSO-d₆) δ ppm: 9.71 (bs, 1H), 8.76 (bs, 1H), 8.58 (d, 1H), 8.56 (s, 1H), 8.44 (d, 1H), 8.15 (s, 2H), 6.92 (s, 1H), 3.81 (m, 1H), 1.49 (d, 6H).

Powder X-Ray Diffraction Peaks of Example 4:

| Angle | d Value | Rel. Intensity |
|---|---|---|
| °2θ | Å | % |
| 7.27 | 12.14 | 5.60 |
| 12.46 | 7.10 | 14.30 |
| 14.54 | 6.09 | 100.00 |
| 18.24 | 4.86 | 31.20 |
| 21.30 | 4.17 | 8.00 |
| 21.90 | 4.06 | 89.40 |
| 24.24 | 3.67 | 9.10 |
| 27.42 | 3.25 | 18.20 |

### EXAMPLE 5

### (S)-1-(5-chloro-6-(2H-1,2,3-triazol-2-yl)pyridin-3-yl)-3-(2-chloro-7-(1-methoxyethyl)pyrazolo[1,5-a]pyrimidin-6-yl)urea

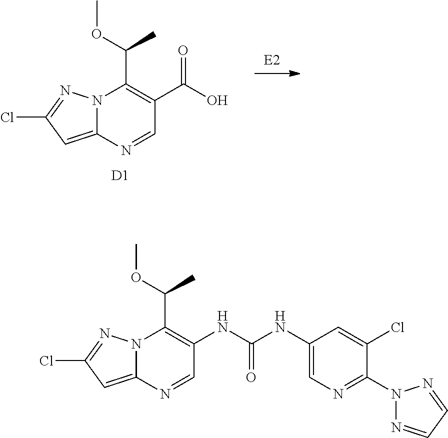

To a solution of (S)-2-chloro-7-(1-methoxyethyl)pyrazolo[1,5-a]pyrimidine-6-carboxylic acid (2.0 g, 7.35 mmol) (D1) in 1,4-dioxane (16 ml) were added DPPA (1.90 ml, 8.82 mmol) and Et₃N (5.12 ml, 36.8 mmol). The reaction mixture was stirred at RT for 30 min. Then, 5-chloro-6-(2H-1,2,3-triazol-2-yl)pyridin-3-amine (1.87 g, 9.56 mmol) was added and reaction mixture was stirred at 100° C. for 2 h. The mixture was partitioned between AcOEt and saturated aqueous NaHCO₃ and the phases were separated. The organic layer was dried over Na₂SO₄, filtered and concentrated. The crude material was purified by flash column chromatography on silica gel (cyclohexane/AcOEt: 1/0 to 0/1), followed by reverse phase HPLC (Method A). The product was then taken up in acetonitrile and heated until disolution. After cooling to RT the precipitate was collected by filtration, washed and dried to afford (S)-1-(5-chloro-6-(2H-1,2,3-triazol-2-yl)pyridin-3-yl)-3-(2-chloro-7-(1-methoxyethyl)pyrazolo[1,5-a]pyrimidin-6-yl)urea. M/z=448-450 [M+H]+, Rt=3.99 min (HPLC Method C1), ¹H NMR (400 MHz, DMSO-d₆) δ ppm: 10.33 (bs, 1H), 8.95 (s, 1H), 8.60 (bs, 1H), 8.54 (d, 1H), 8.49 (d, 1H), 8.16 (s, 2H), 6.94 (s, 1H), 5.42 (q, 1H), 3.33 (s, 3H), 1.59 (d, 3H).

Powder X-Ray Diffraction Peaks of Example 5:

| Angle | d Value | Rel. Intensity |
|---|---|---|
| °2θ | Å | % |
| 8.20 | 10.77 | 31.00 |
| 10.71 | 8.26 | 22.30 |
| 12.87 | 6.87 | 100.00 |
| 13.27 | 6.66 | 69.70 |
| 16.83 | 5.26 | 22.20 |
| 20.00 | 4.44 | 31.40 |
| 20.65 | 4.30 | 29.10 |
| 23.25 | 3.82 | 47.20 |
| 27.25 | 3.27 | 55.70 |
| 29.10 | 3.07 | 56.30 |

Solubility of Example 5:

| Media | Solubility (mg/mL) [pH] |
|---|---|
| pH 1.2 | 0.0038 [1.17] |
| pH 3.0 | 0.0052 [3.14] |
| pH 4.7 | 0.0048 [4.71] |
| pH 7.4 | 0.0040 [7.34] |
| pH 9.0 | 0.0055 [8.09] |
| Water | 0.0050 [7.39] |
| Simulated Gastric Fluid | 0.0168 [2.55] |
| (SGF) | |
| Fasted state simulated | 0.0044 [6.59] |
| intestinal fluid (FaSSIF) | |
| Fed state intestinal fluid | 0.0082 [6.87] |
| (FeSSIF) | |

### EXAMPLE 6

### (S)-1-(5-cyano-6-methoxypyridin-3-yl)-3-(2-fluoro-7-(1-methoxyethyl) pyrazolo[1,5-a]pyrimidin-6-yl)urea

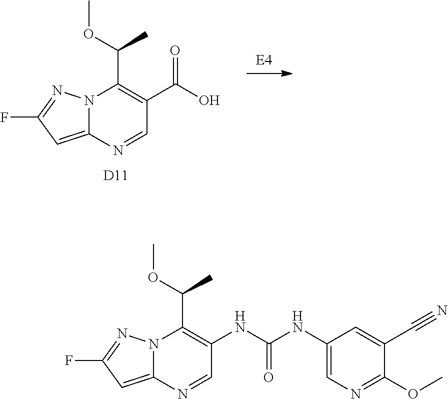

To a solution of (S)-2-fluoro-7-(1-methoxyethyl)pyrazolo[1,5-a]pyrimidine-6-carboxylic acid (50 mg, 0.199 mmol) in dioxane (0.8 ml) were added DPPA (0.053 ml, 0.238 mmol) and Et₃N (0.083 ml, 0.596 mmol). The reaction mixture was stirred at RT for 30 min. Then, 5-amino-2-methoxynicotinonitrile (35.5 mg, 0.238 mmol) was added and the reaction mixture was stirred at 100° C. for 20 min. The mixture was cooled to RT, diluted with AcOEt and washed with aq. sat. NaHCO₃ and brine. The organic layer was dried over Na₂SO₄, filtered, concentrated and purified by flash column chromatography on silica gel (cyclohexane/AcOEt: 10/0 to 5/5) to afford after recrystallisation in ACN, (S)-1-(5-cyano-6-methoxypyridin-3-yl)-3-(2-fluoro-7-(1-methoxyethyl) pyrazolo[1,5-a]pyrimidin-6-yl)urea. M/z=386 [M+H]+, Rt=3.81 min (HPLC Method C1), ¹H NMR (600 MHz, DMSO-d₆) δ ppm: 9.71 (s, 1H), 8.87 (s, 1H), 8.41 (m, 3H), 6.53 (d, 1H), 5.33 (q, 1H), 3.97 (s, 3H), 3.30 (s, 3H), 1.56 (d, 3H).

In analogy to Example 1 the following examples were prepared:

| Ex | Structure | Name | Analytics |
|---|---|---|---|
| 7 | 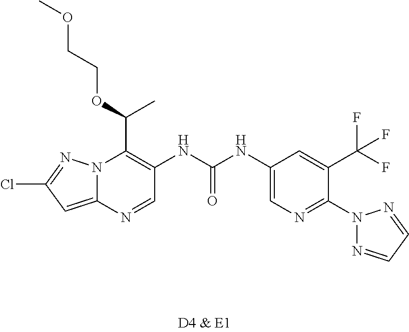 [SMILES: COCCO[C@@H](C)c1c(NC(=O)Nc2cnc(-n3nccn3)c(C(F)(F)F)c2)cnc2cc(Cl)nn12] | (S)-1-(6-(2H-1,2,3- triazol-2-yl)-5- (trifluoromethyl) pyridin- 3-yl)-3-(2-chloro- 7-(1-(2- methoxyethoxy)ethyl) pyrazolo[1,5- a]pyrimidin-6-yl)urea | M/z = 526-528 [M + H]+, Rt = 4.55 min (HPLC Method C1), ¹H NMR (400 MHz, DMSO-d₆) δ ppm: 10.46 (bs, 1H), 8.97 (s, 1H), 8.86 (d, 1H), 8.72 (d 1H), 8.65 (s, 1H), 8.17 (s, 2H), 6.94 (s, 1H), 5.56 (q, 1H), 3.67 (m, 1H), 3.54 (m, 1H), 3.49- 3.41 (m, 2H), 3.09 (s, 3H), 1.60 (d, 3H). |
| 8 | 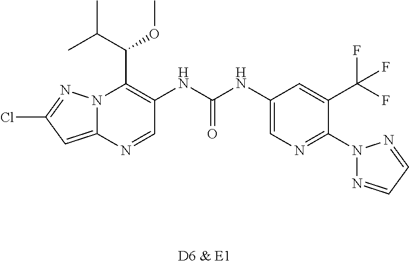 [SMILES: CO[C@H](c1c(NC(=O)Nc2cnc(-n3nccn3)c(C(F)(F)F)c2)cnc2cc(Cl)nn12)C(C)C] | (S)-1-(6-(2H-1,2,3- triazol-2-yl)-5- (trifluoromethyl) pyridin-3-yl)-3- (2-chloro- 7-(1-methoxy-2- methylpropyl)pyrazolo [1,5-a]pyrimidin-6- yl)urea | M/z = 510-512 [M + H]+, Rt = 5.44 min (HPLC Method C1), ¹H NMR (600 MHz, DMSO-d₆) δ ppm: 10.54 (s, 1H), 8.98 (s, 1H), 8.82 (d, 1H), 8.71 (d, 1H), 8.51 (s, 1H), 8.17 (s, 2H), 6.94 (s, 1H), 5.24-4.87 (m, 1H), 3.36 (s, 3H), 2.37 (m, 1H), 1.08 (d, 3H), 0.81 (d, 3H). |
| 9 | 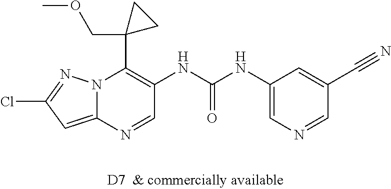 [SMILES: COCC1(c2c(NC(=O)Nc3cncc(C#N)c3)cnc3cc(Cl)nn23)CC1] | 1-(2-chloro-7-(1- (methoxymethyl) cyclopropyl)pyrazolo[1,5- a]pyrimidin-6-yl)-3- (5-cyanopyridin-3- yl)urea | M/z = 398-400 [M + H]+, Rt = 3.73 min (HPLC Method C1), ¹H NMR (400 MHz, DMSO-d₆) δ ppm: 10.20 (s, 1H), 8.84 (d, 1H), 8.78 (s, 1H), 8.65 (s, 1H), 8.42 (s, 1H), 8.15 (s, 1H), 6.91 (s, 1H), 3.60 (s, 2H), 3.23 (s, 3H), 1.24 (m, 2H), 1.01 (m, 2H). |
| 10 | 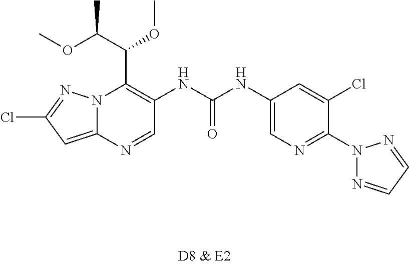 [SMILES: CO[C@@H](C)[C@H](OC)c1c(NC(=O)Nc2cnc(-n3nccn3)c(Cl)c2)cnc2cc(Cl)nn12] | 1-(5-chloro-6-(2H- 1,2,3-triazol-2- yl)pyridin-3-yl)-3-(2- chloro-7-((1R, 2S)- 1,2- dimethoxypropyl) pyrazolo[1,5- a]pyrimidin-6-yl) urea | M/z = 492-494 [M + H]+, Rt = 4.33 min (HPLC Method C1), ¹H NMR (400 MHz, DMSO-d₆) δ ppm: 10.37 (s, 1H), 8.90 (s, 1H), 8.55 (d, 1H), 8.51-8.42 (m, 2H), 8.15 (s, 2H), 6.93 (s, 1H), 5.39 (d, 1H), 3.96 (m, 1H), 3.36 (s, 3H), 3.21 (s, 3H), 1.17 (d, 3H). |
| 11 | 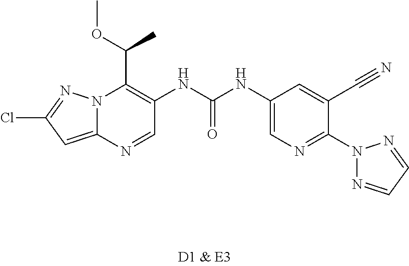 [SMILES: CO[C@@H](C)c1c(NC(=O)Nc2cnc(-n3nccn3)c(C#N)c2)cnc2cc(Cl)nn12] | (S)-1-(2-chloro-7-(1- methoxyethyl) pyrazolo[1,5-a] pyrimidin-6- yl)-3-(5-cyano-6- (2H-1,2,3-triazol-2- yl)pyridin-3-yl)urea | M/z = 439-441 [M + H]+, Rt = 3.86 min (HPLC Method C1), ¹H NMR (400 MHz, DMSO-d₆) δ ppm: 10.32 (bs, 1H), 8.95 (s, 1H), 8.83 (s, 1H), 8.68 (s, 1H), 8.61 (bs, 1H), 8.27 (s, 2H), 6.94 (s, 1H), 5.42 (q, 1H), 3.35 (s, 3H), 1.59 (d, 3H). |
| 12 | 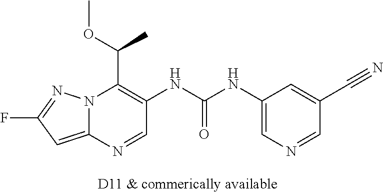 [SMILES: CO[C@@H](C)c1c(NC(=O)Nc2cncc(C#N)c2)cnc2cc(F)nn12] | (S)-1-(5- cyanopyridin-3-yl)-3- (2-fluoro-7-(1- methoxyethyl) pyrazolo[1,5-a] pyrimidin-6-yl)urea | M/z = 356 [M + H]+, Rt = 3.02 min (HPLC Method C1), ¹H NMR (400 MHz, DMSO-d₆) δ ppm: 10.03 (s, 1H), 8.87 (s, 1H), 8.84 (s, 1H), 8.64 (d, 1H), 8.48 (s, 1H), 8.42 (q, 1H), 6.55 (dd, 1H), 5.33 (q, 1H), 3.31 (s, 3H), 1.56 (d, 3H). |
| 13 | 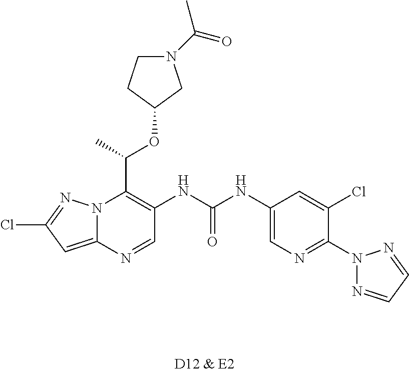 [SMILES: CC(=O)N1CC[C@@H](O[C@@H](C)c2c(NC(=O)Nc3cnc(-n4nccn4)c(Cl)c3)cnc3cc(Cl)nn23)C1] | 1-(7-((S)-1-(((R)-1- acetylpyrrolidin-3- yl)oxy)ethyl)-2- chloropyrazolo[1,5- a]pyrimidin-6-yl)-3- (5-chloro-6-(2H- 1,2,3-triazol-2- yl)pyridin-3-yl)urea | M/z = 545-547 [M + H]+, Rt = 3.53 min (HPLC Method C1), ¹H NMR (400 MHz, DMSO-d₆) δ ppm: 10.22 (s, 1H), 8.90 (d, 1H), 8.57 (s, 1H), 8.53-8.42 (m 2H), 8.15 (s, 2H), 6.95 (d, 1H), 5.62 (m, 1H), 4.14-4.03 (m, 1H), 3.51-3.16 (m, 4H), 2.27-2.09 (m, 1H), 1.95-1.78 (m, 1H), 1.88- 1.73 (m, 3H), 1.62 (m, 3H) |
| 14 | 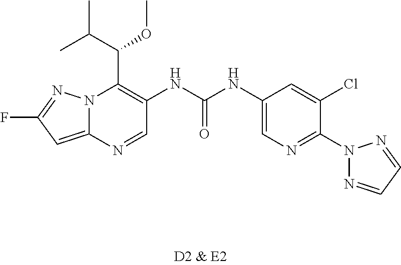 [SMILES: CO[C@H](c1c(NC(=O)Nc2cnc(-n3nccn3)c(Cl)c2)cnc2cc(F)nn12)C(C)C] | (S)-1-(5-chloro-6- (2H-1,2,3-triazol-2- yl)pyridin-3-yl)-3-(2- fluoro-7-(1-methoxy- 2- methylpropyl)pyrazolo [1,5-a]pyrimidin-6- yl)urea | M/z = 460-462 [M + H]+, Rt = 4.75 min (HPLC Method C1), ¹H NMR (600 MHz, DMSO-d₆) δ ppm: 10.34 (s, 1H), 8.91 (s, 1H), 8.53 (d, 1H), 8.48 (d, 1H), 8.42 (s, 1H), 8.15 (s, 2H), 6.55 (d, 1H), 4.99 (d, 1H), 3.36 (s, 3H), 2.38 (m, 1H), 1.08 (d, 3H), 0.80 (d, 3H). |
| 15 | 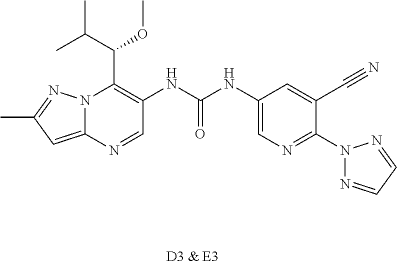 [SMILES: CO[C@H](c1c(NC(=O)Nc2cnc(-n3nccn3)c(C#N)c2)cnc2cc(C)nn12)C(C)C] | (S)-1-(5-cyano-6- (2H-1,2,3-triazol-2- yl)pyridin-3-yl)-3-(7- (1-methoxy-2- methylpropyl)-2- methylpyrazolo[1,5- a]pyrimidin-6-yl)urea | M/z = 447 [M + H]+, Rt = 3.97 min (HPLC Method C1), ¹H NMR (400 MHz, DMSO-d₆) δ ppm: 10.33 (s, 1H), 8.81 (d, 1H), 8.77 (s, 1H), 8.68 (d, 1H), 8.36 (s, 1H), 8.27 (s, 2H), 6.56 (s, 1H), 5.18 (d, 1H), 3.33 (s, 3H), 2.44 (m, 4H), 1.08 (d, 3H), 0.79 (d, 3H). |
| 16 | 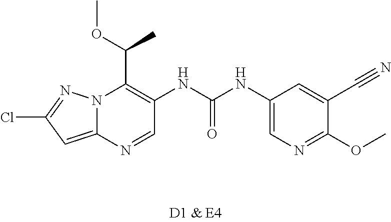 [SMILES: COc1ncc(NC(=O)Nc2cnc3cc(Cl)nn3c2[C@H](C)OC)cc1C#N] | (S)-1-(2-chloro-7-(1- methoxyethyl)pyrazolo [1,5-a]pyrimidin-6- yl)-3-(5-cyano-6- methoxypyridin-3- yl)urea | M/z = 402-404 [M + H]+, Rt = 4.17 min (HPLC Method C1), ¹H NMR (400 MHz, DMSO-d₆) δ ppm: 9.77 (s, 1H), 8.93 (s, 1H), 8.53- 8.29 (m, 3H), 6.91 (s, 1H), 5.41 (q, 1H), 3.97 (s, 3H), 3.33 (s, 3H), 1.57 (d, 3H). |
| 17 | 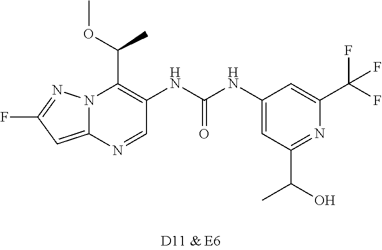 [SMILES: CO[C@@H](C)c1c(NC(=O)Nc2cc(C(C)O)nc(C(F)(F)F)c2)cnc2cc(F)nn12] | 1-(2-fluoro-7-((S)-1- methoxyethyl)pyrazolo [1,5-a]pyrimidin-6- yl)-3-(2-(1- hydroxyethyl)-6- (trifluoromethyl)pyridin- 4-yl)urea | M/z = 443 [M + H]+, Rt = 4.10 min (HPLC Method C1), ¹H NMR (400 MHz, DMSO-d₆) δ ppm: 10.38 (s, 1H), 8.90 (s, 1H), 8.47 (s, 1H), 7.96 (t, 1H), 7.74 (d, 1H), 6.56 (d, 1H), 5.57 (d, 1H), 5.34 (q, 1H), 4.80- 4.65 (m, 1H), 1.56 (d, 3H), 1.36 (d, 3H). CH₃ hidden under solvent peak |
| 18 | 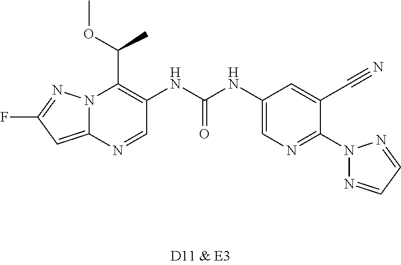 [SMILES: CO[C@@H](C)c1c(NC(=O)Nc2cnc(-n3nccn3)c(C#N)c2)cnc2cc(F)nn12] | (S)-1-(5-cyano-6- (2H-1,2,3-triazol-2- yl)pyridin-3-yl)-3-(2- fluoro-7-(1- methoxyethyl)pyrazolo [1,5-a]pyrimidin-6- yl)urea | M/z = 423 [M + H]+, Rt = 3.52 min (HPLC Method C1), ¹H NMR (600 MHz, DMSO-d₆) δ ppm: 10.26 (s, 1H), 8.90 (s, 1H), 8.83 (d, 1H), 8.67 (d, 1H), 8.58 (s, 1H), 8.27 (s, 2H), 6.56 (d, 1H), 5.34 (q, 1H), 3.33 (s, 3H), 1.58 (d, 3H). |
| 19 | 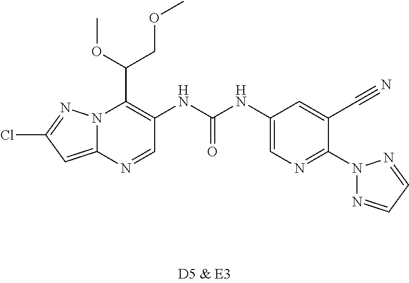 [SMILES: COCC(OC)c1c(NC(=O)Nc2cnc(-n3nccn3)c(C#N)c2)cnc2cc(Cl)nn12] | 1-(2-chloro-7-(1,2- dimethoxyethyl)pyrazolo [1,5-a]pyrimidin- 6-yl)-3-(5-cyano-6- (2H-1,2,3-triazol-2- yl)pyridin-3-yl)urea | M/z = 469-471 [M + H]+, Rt = 3.67 min (HPLC Method C1), ¹H NMR (400 MHz, DMSO-d₆) δ ppm: 10.44 (s, 1H), 8.95 (s, 1H), 8.83 (d, 1H), 8.68 (d, 1H), 8.49 (s, 1H), 8.28 (s, 2H), 6.96 (s, 1H), 5.51 (dd, 1H), 3.93- 3.72 (m, 2H), 3.36 (s, 3H), 3.29 (s, 3H). |
| 20 | 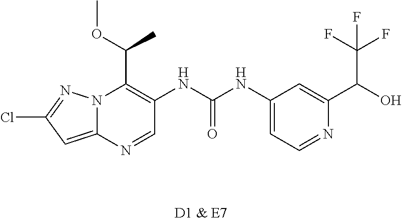 [SMILES: CO[C@@H](C)c1c(NC(=O)Nc2ccnc(C(O)C(F)(F)F)c2)cnc2cc(Cl)nn12] | 1-(2-chloro-7-((S)-1- methoxyethyl)pyrazolo [1,5-a]pyrimidin-6- yl)-3-(2-(2,2,2- trifluoro-1- hydroxyethyl)pyridin- 4-yl)urea | M/z = 445-447 [M + H]+, Rt = 2.98 min (HPLC Method C1), ¹H NMR (400 MHz, DMSO-d₆) δ ppm: 10.14 (s, 1H), 8.94 (s, 1H), 8.47- 8.32 (m, 2H), 7.73 (s, 1H), 7.52 (dd, 1H), 7.02-6.87 (m, 2H), 5.42 (q, 1H), 5.06 (m, 1H), 1.56 (d, 3H). CH₃ hidden under solvent peak |
| 21 | 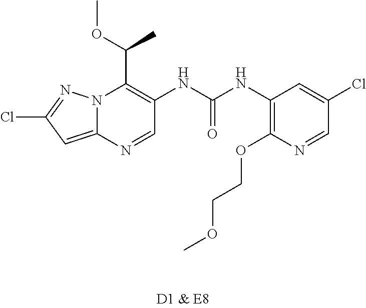 [SMILES: COCCOc1ncc(Cl)cc1NC(=O)Nc1cnc2cc(Cl)nn2c1[C@H](C)OC] | (S)-1-(5-chloro-2-(2- methoxyethoxy)pyridin- 3-yl)-3-(2-chloro- 7-(1-methoxyethyl)- pyrazolo[1,5- a]pyrimidin-6-yl)urea | M/z = 455-457 [M + H]+, Rt = 1.20 min (UPLC Method B2), ¹H NMR (400 MHz, DMSO-d₆) δ ppm: 9.10 (s, 1H), 8.97 (s, 1H), 8.74 (s, 1H), 8.45 (d, 1H), 7.82 (d, 1H), 6.92 (s, 1H), 5.37 (q, 1H), 4.57 (t, 2H), 3.74 (t, 2H), 3.25 (s, 3H), 1.59 (d, 3H). OCH₃ hidden under solvent peak |
| 22 | 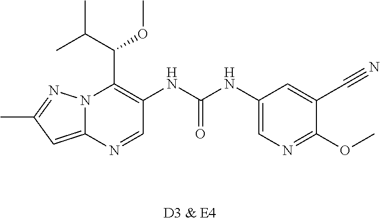 | (S)-1-(5-cyano-6- methoxypyridin-3- yl)-3-(7-(1-methoxy- 2-methylpropyl)-2- methylpyrazolo[1,5- a]pyrimidin-6-yl)urea | M/z = 410 [M + H]+, Rt = 4.24 min (HPLC Method C1), ¹H NMR (400 MHz, DMSO-d₆) δ ppm: 9.78 (s, 1H), 8.74 (s, 1H), 8.44 (d, 1H), 8.36 (d, 1H), 8.16 (s, 1H), 6.53 (s, 1H), 5.16 (m, 1H), 3.96 (s, 3H), 3.33 (s, 3H), 2.42 (s, 4H), 1.06 (d, 3H), 0.78 (d, 3H). |
| 23 | 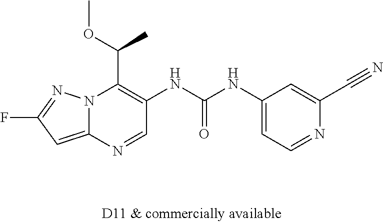 [SMILES: CO[C@@H](C)c1c(NC(=O)Nc2ccnc(C#N)c2)cnc2cc(F)nn12] | (S)-1-(2- cyanopyridin-4-yl)-3- (2-fluoro-7-(1- methoxyethyl)pyrazolo [1,5-a]pyrimidin-6- yl)urea | M/z = 356 [M + H]+, Rt = 3.39 min (HPLC Method C1), ¹H NMR (400 MHz, DMSO-d₆) δ ppm: 10.28 (s, 1H), 8.85 (s, 1H), 8.54 (m, 2H), 8.06 (d, 1H), 7.68 (dd, 1H), 6.56 (d, 1H), 5.32 (q, 1H), 3.30 (s, 3H), 1.56 (d, 3H). |
| 24 | 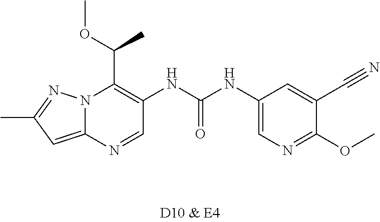 | (S)-1-(5-cyano-6- methoxypyridin-3- yl)-3-(7-(1- methoxyethyl)-2- methylpyrazolo[1,5- a]pyrimidin-6-yl)urea | M/z = 382 [M + H]+, Rt = 3.42 min (HPLC Method C1), ¹H NMR (400 MHz, DMSO-d₆) δ ppm: 9.70 (s, 1H), 8.74 (s, 1H), 8.46 (d, 1H), 8.36 (d, 1H), 8.28 (s, 1H), 6.54 (s, 1H), 5.50 (q, 1H), 3.97 (s, 3H), 3.30 (s, 3H), 2.43 (s, 3H), 1.56 (d, 3H). |
| 25 | 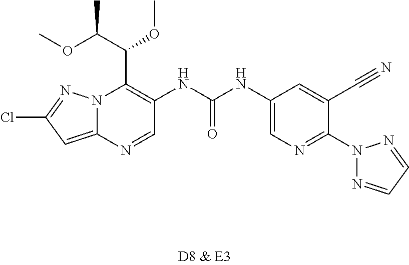 [SMILES: CO[C@@H](C)[C@H](OC)c1c(NC(=O)Nc2cnc(-n3nccn3)c(C#N)c2)cnc2cc(Cl)nn12] | 1-(2-chloro-7- ((1R, 2S)-1,2- dimethoxypropyl) pyrazolo[1,5- a]pyrimidin-6-yl)-3- (5-cyano-6-(2H- 1,2,3-triazol-2- yl)pyridin-3-yl)urea | M/z = 483-485 [M + H]+, Rt = 4.00 min (HPLC Method C1), ¹H NMR (400 MHz, DMSO-d₆) δ ppm: 10.40 (s, 1H), 8.90 (s, 1H), 8.83 (d, 1H), 8.68 (d, 1H), 8.47 (s, 1H), 8.28 (s, 2H), 6.94 (s, 1H), 5.39 (d, 1H), 3.96 (p, 1H), 3.36 (s, 3H), 3.21 (s, 3H), 1.18 (d, 3H). |
| 26 | 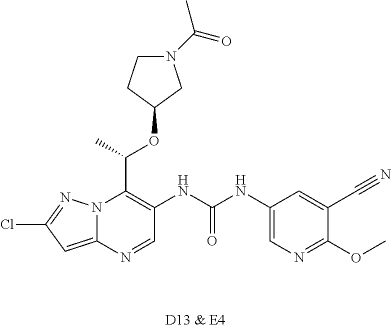 [SMILES: COc1ncc(NC(=O)Nc2cnc3cc(Cl)nn3c2[C@H](C)O[C@H]2CCN(C(C)=O)C2)cc1C#N] | 1-(7-((S)-1-(((S)-1- acetylpyrrolidin-3- yl)oxy)ethyl)-2- chloropyrazolo[1,5- a]pyrimidin-6-yl)-3- (5-cyano-6- methoxypyridin-3- yl)urea | M/z = 499-501 [M + H]+, Rt = 3.44 min (HPLC Method C1), ¹H NMR (400 MHz, DMSO-d₆) δ ppm: 9.62 (s, 1H), 8.86 (d, 1H), 8.47 (d, 1H), 8.35 (d, 1H), 8.30 (d, 1H), 6.93 (s, 1H), 5.63- 5.51 (m, 1H), 4.16-4.06 (m, 1H), 3.97 (s, 3H), 3.68- 3.18 (m, 4H), 1.95-1.72 (m, 5H), 1.61-1.58 (m, 3H) |
| 27 | 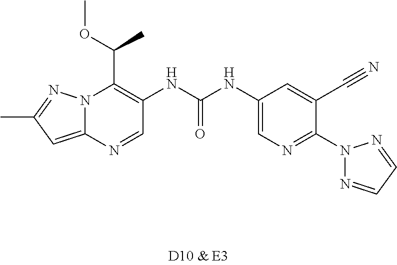 [SMILES: CO[C@@H](C)c1c(NC(=O)Nc2cnc(-n3nccn3)c(C#N)c2)cnc2cc(C)nn12] | (S)-1-(5-cyano-6- (2H-1,2,3-triazol-2- yl)pyridin-3-yl)-3-(7- (1-methoxyethyl)-2- methylpyrazolo[1,5- a]pyrimidin-6-yl)urea | M/z = 419 [M + H]+, Rt = 3.18 min (HPLC Method C1), ¹H NMR (400 MHz, DMSO-d₆) δ ppm: 10.26 (s, 1H), 8.83 (d, 1H), 8.76 (s, 1H), 8.68 (d, 1H), 8.49 (s, 1H), 8.27 (s, 2H), 6.57 (s, 1H), 5.51 (q, 1H), 3.32 (s, 3H), 2.45 (s, 3H), 1.58 (d, 3H). |
| 28 | 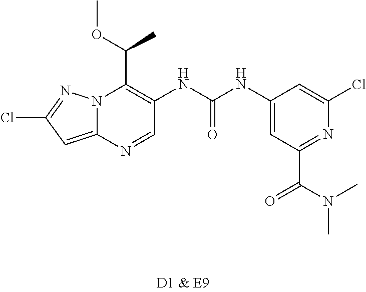 [SMILES: CO[C@@H](C)c1c(NC(=O)Nc2cc(Cl)nc(C(=O)N(C)C)c2)cnc2cc(Cl)nn12] | (S)-6-chloro-4-(3-(2- chloro-7-(1- methoxyethyl)pyrazolo [1,5-a]pyrimidin-6- yl)ureido)-N, N- dimethylpicolinamide | [d], M/z = 452-454 [M + H]+, Rt = 0.98 min (UPLC Method B2), ¹H NMR (400 MHz, DMSO-d₆) δ ppm: 10.31 (s, 1H), 8.89 (s, 1H), 8.57 (s, 1H), 7.67 (d, 1H), 7.53 (d, 1H), 6.94 (s, 1H), 5.40 (q, 1H), 2.99 (s, 3H), 2.97 (s, 3H), 1.57 (d, 3H). |
| 29 | 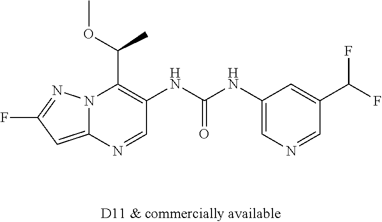 [SMILES: CO[C@@H](C)c1c(NC(=O)Nc2cncc(C(F)F)c2)cnc2cc(F)nn12] | (S)-1-(5-(difluoro- methyl)pyridin-3-yl)- 3-(2-fluoro-7-(1- methoxyethyl)- pyrazolo[1,5- a]pyrimidin-6-yl)urea | M/z = 381 [M + H]+, Rt = 2.68 min (HPLC Method C1), ¹H NMR (400 MHz, DMSO-d₆) δ ppm: 9.95 (s, 1H), 8.89 (s, 1H), 8.70 (s, 1H), 8.41 (s, 2H), 8.28 (s, 1H), 7.15 (t, 1H), 6.54 (d, 1H), 5.34 (q, 1H), 1.57 (d, 3H). CH₃ hidden under solvent peak |
| 30 | 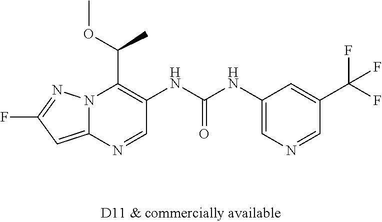 [SMILES: CO[C@@H](C)c1c(NC(=O)Nc2cncc(C(F)(F)F)c2)cnc2cc(F)nn12] | (S)-1-(2-fluoro-7-(1- methoxyethyl)pyrazolo [1,5-a]pyrimidin-6- yl)-3-(5-(trifluoro- methyl)pyridin-3- yl)urea | M/z = 399 [M + H]+, Rt = 3.76 min (HPLC Method C1), ¹H NMR (400 MHz, DMSO-d₆) δ ppm: 10.09 (s, 1H), 8.89 (s, 1H), 8.80 (d, 1H), 8.59 (m, 1H), 8.49 (s, 1H), 8.44 (t, 1H), 6.55 (d, 1H), 5.34 (q, 1H), 3.31 (s, 3H), 1.57 (d, 3H). |
| 31 | 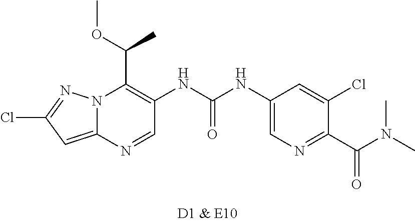 [SMILES: CO[C@@H](C)c1c(NC(=O)Nc2cnc(C(=O)N(C)C)c(Cl)c2)cnc2cc(Cl)nn12] | (S)-3-chloro-5-(3-(2- chloro-7-(1- methoxyethyl)pyrazolo [1,5-a]pyrimidin-6- yl)ureido)-N,N- dimethylpicolinamide | [d], M/z = 452-454 [M + H]+, Rt = 0.90 min (UPLC Method B2), ¹H NMR (400 MHz, DMSO-d₆) δ ppm: 10.11 (s, 1H), 8.92 (s, 1H), 8.52 (s, 2H), 8.25 (d, 1H), 6.93 (s, 1H), 5.41 (q, 1H), 3.01 (s, 3H), 2.78 (s, 3H), 1.58 (d, 3H). CH₃ hidden under solvent peak |
| 32 | 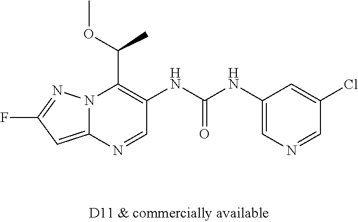 [SMILES: CO[C@@H](C)c1c(NC(=O)Nc2cncc(Cl)c2)cnc2cc(F)nn12] | (S)-1-(5-chloro- pyridin-3-yl)-3-(2- fluoro-7-(1- methoxyethyl)pyrazolo [1,5-a]pyrimidin-6- yl)urea | M/z = 365-367 [M + H]+, Rt = 3.08 min (HPLC Method C1), ¹H NMR (400 MHz, DMSO-d₆) δ ppm: 9.93 (bs, 1H), 8.87 (s, 1H), 8.50 (d, 1H), 8.43 (s, 1H), 8.26 (d, 1H), 8.19 (t, 1H), 6.55 (d, 1H), 5.33 (q, 1 H), 3.30 (s, 3H), 1.56 (d, 3H). |
| 33 | 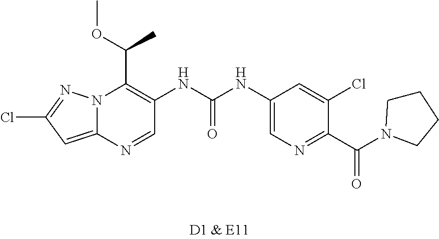 [SMILES: CO[C@@H](C)c1c(NC(=O)Nc2cnc(C(=O)N3CCCC3)c(Cl)c2)cnc2cc(Cl)nn12] | (S)-1-(5-chloro-6- (pyrrolidine-1- carbonyl)pyridin-3- yl)-3-(2-chloro-7-(1- methoxyethyl)pyrazolo [1,5-a]pyrimidin-6- yl)urea | [d], M/z = 478-480 [M + H]+, Rt = 0.96 min (UPLC Method B2), ¹H NMR (400 MHz, MeOH-d₆) δ ppm: 9.03 (s, 1H), 8.55 (d, 1H), 8.36 (d, 1H), 6.71 (s, 1H), 5.58 (q, 1H), 3.65 (t, 2H), 3.48 (s, 3H), 3.30 (t, 2H), 2.09-1.91 (m, 4H), 1.65 (d, 3H). |
| 34 | 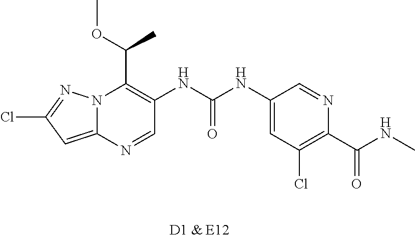 [SMILES: CNC(=O)c1ncc(NC(=O)Nc2cnc3cc(Cl)nn3c2[C@H](C)OC)cc1Cl] | (S)-3-chloro-5-(3-(2- chloro-7-(1- methoxyethyl)pyrazolo [1,5-a]pyrimidin-6- yl)ureido)-N- methylpicolinamide | M/z = 438-440 [M + H]+, Rt = 0.88 min (UPLC Method B2), ¹H NMR (400 MHz, DMSO-d₆) δ ppm: 10.23 (s, 1H), 8.92 (s, 1H), 8.54 (s, 2H), 8.54-8.45 (m, 2H), 8.22 (s, 1H), 6.94 (s, 1H), 5.41 (q, 1H), 2.75 (d, 3H), 1.60 (d, 3H). 2 x CH₃ hidden under solvent peaks |
| 35 | 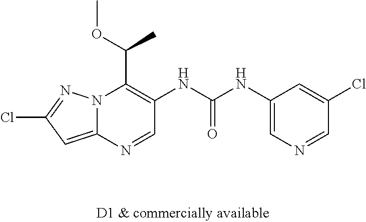 [SMILES: CO[C@@H](C)c1c(NC(=O)Nc2cncc(Cl)c2)cnc2cc(Cl)nn12] | (S)-1-(2-chloro-7-(1- methoxyethyl)pyrazolo [1,5-a]pyrimidin-6- yl)-3-(5- chloropyridin-3- yl)urea | [d], M/z = 381-383 [M + H]+, Rt = 1.00 min (UPLC Method B2), ¹H NMR (400 MHz, DMSO-d₆) δ ppm: 9.98 (bs, 1H), 8.92 (s, 1H), 8.50 (d, 1H), 8.46 (bs, 1H), 8.26 (d, 1H), 8.20 (t, 1H), 6.92 (s, 1H), 5.41 (q, 1H), 1.57 (d, 3H). CH₃ hidden under solvent peak |
| 36 | 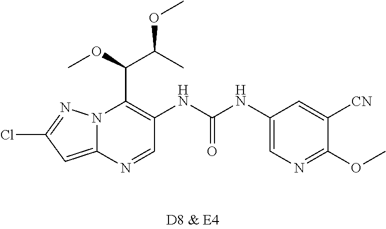 [SMILES: COc1ncc(NC(=O)Nc2cnc3cc(Cl)nn3c2[C@@H](OC)[C@H](C)OC)cc1C#N] | 1-(2-chloro-7- ((1R, 2S)-1,2- dimethoxypropyl) pyrazolo[1,5- a]pyrimidin-6-yl)-3- (5-cyano-6- methoxypyridin-3- yl)urea | M/z = 446-448 [M + H]+, Rt = 1.10 min (UPLC Method B2), ¹H NMR (400 MHz, DMSO-d₆) δ ppm: 9.86 (s, 1H), 8.89 (s, 1H), 8.48 (d, 1H), 8.37 (d, 1H), 8.30 (s, 1H), 6.92 (s, 1H), 5.39 (d, 1H), 3.98 (s, 3H), 3.93 (dq, 1H), 3.35 (s, 3H), 3.21 (s. 3H), 1.18 (d, 3H). |
| 37 | 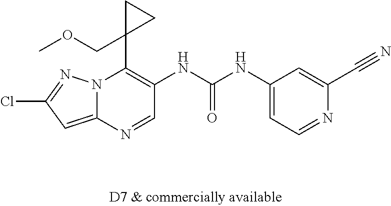 [SMILES: COCC1(c2c(NC(=O)Nc3ccnc(C#N)c3)cnc3cc(Cl)nn23)CC1] | 1-(2-chloro-7-(1- (methoxymethyl) cyclopropyl)pyrazolo [1,5-a]pyrimidin-6-yl)-3- (2-cyanopyridin-4- yl)urea | M/z = 398-400 [M + H]+, Rt = 3.82 min (UPLC Method B7), ¹H NMR (400 MHz, DMSO-d₆) δ ppm: 10.46 (bs, 1H), 8.79 (s, 1H), 8.56 (d, 1H), 8.23 (s, 1H), 8.09 (d, 1H), 7.70 (dd, 1H), 6.93 (s, 1H), 3.61 (bs, 2H), 3.24 (s, 3H), 1.25 (t, 2H), 1.01 (t, 2H). |

[d]: The reaction mixture was stirred at 80° C. after addition of aniline.

### EXAMPLE 38

### (S)-1-(7-(1-aminoethyl)-2-chloropyrazolo[1,5-a]pyrimidin-6-yl)-3-(5-chloro-6-(2H-1,2,3-triazol-2-yl)pyridin-3-yl)urea

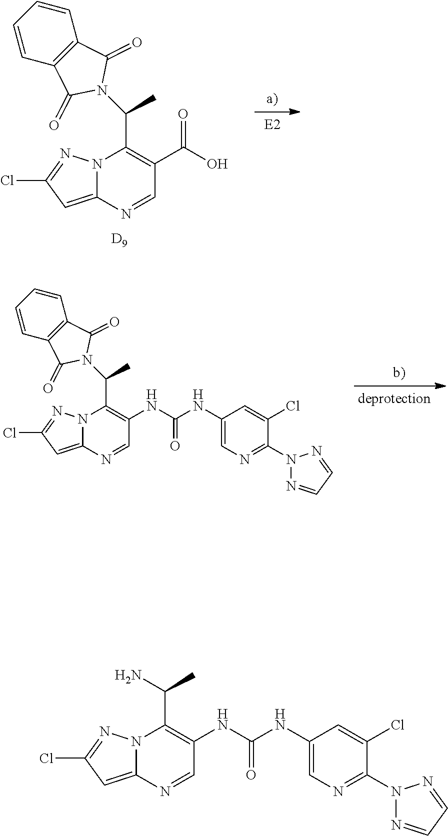

### a) (S)-1-(5-chloro-6-(2H-1,2,3-triazol-2-yl)pyridin-3-yl)-3-(2-chloro-7-(1-(1,3-dioxoisoindolin-2-yl)ethyl)pyrazolo[1,5-a]pyrimidin-6-yl)urea

(S)-1-(5-chloro-6-(2H-1,2,3-triazol-2-yl)pyridin-3-yl)-3-(2-chloro-7-(1-(1,3-dioxoisoindolin-2-yl)ethyl)pyrazolo[1,5-a]pyrimidin-6-yl)urea was prepared analogously as described in example 1 using (S)-2-chloro-7-(1-(1,3-dioxoisoindolin-2-yl)ethyl)pyrazolo[1,5-a]-pyrimidine-6-carboxylic acid instead of (S)-2-chloro-7-(1-methoxyethyl)pyrazolo[1,5-a]pyrimidine-6-carboxylic acid and using 5-chloro-6-(2H-1,2,3-triazol-2-yl)pyridin-3-amine instead of 6-(2H-1,2,3-triazol-2-yl)-5-(trifluoromethyl)pyridin-3-amine. M/z=563-565 [M+H]+, Rt=1.09 min (UPLC Method B1).

### b) (S)-1-(7-(1-aminoethyl)-2-chloropyrazolo[1,5-a]pyrimidin-6-yl)-3-(5-chloro-6-(2H-1,2,3-triazol-2-yl)pyridin-3-yl)urea

A solution of (S)-1-(5-chloro-6-(2H-1,2,3-triazol-2-yl)pyridin-3-yl)-3-(2-chloro-7-(1-(1,3-dioxoisoindolin-2-yl)ethyl)pyrazolo[1,5-a]pyrimidin-6-yl)urea (86 mg, 0.15 mmol) in hydrazine 1M in THF (763 μl, 0.76 mmol) was stirred at RT overnight. The reaction was then filtered, concentrated and purified by preparative HPLC (Method A1), fractions were combined and extracted with AcOEt and sat. aq. NaHCO₃. The organic layer was dried over Na₂SO₄, filtered, concentrated and dried under HV to afford (S)-1-(7-(1-aminoethyl)-2-chloropyrazolo[1,5-a]pyrimidin-6-yl)-3-(5-chloro-6-(2H-1,2,3-triazol-2-yl)pyridin-3-yl) urea. M/z=433-435 [M+H]+, Rt=2.40 min (HPLC Method C1), ¹H NMR (400 MHz, DMSO-d₆) δ ppm: 8.95 (s, 1H), 8.59 (d, 1H), 8.48 (s, 1H), 8.15 (s, 2H), 6.88 (s, 1H), 5.49 (bs, 2H), 5.06 (q, 1H), 1.47 (d, 3H).

### EXAMPLE 39

### (S)-1-(5-cyanopyridin-3-yl)-3-(7-(1-hydroxyethyl)-2-methylpyrazolo [1,5-a]pyrimidin-6-yl)urea

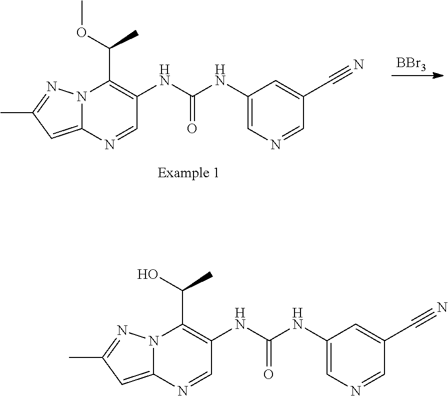

A suspension of (S)-1-(5-cyanopyridin-3-yl)-3-(7-(1-methoxyethyl)-2-methylpyrazolo [1,5-a]pyrimidin-6-yl)urea (50.0 mg, 0.14 mmol) in DCM (2.0 ml) was cooled to 0° C. and boron tribromide (1 M solution in DCM, 0.85 ml) was added dropwise. The resulting suspension was stirred for 5 h at 0° C. and additional 16 h at RT. The reaction mixture was poured onto ice-water and extracted with ethylacetate. The organic extracts were washed with aq. 1N HCl and brine, dried and concentrated. Preparative HPLC (Method A2), followed by SFC purification (Method A5) yielded the title compound. M/z=338 [M+H]+, Rt=2.21 min (UPLC Method B7), ¹H NMR (400 MHz, DMSO-d₆) δ ppm:

### EXAMPLE 40

### (S)-1-(2-(difluoromethyl)pyridin-4-yl)-3-(2-fluoro-7-(1-hydroxyethyl) pvrazolo[1,5-a]pyrimidin-6-yl)urea

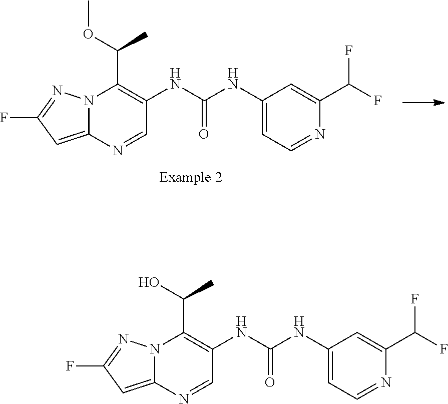 [SMILES: CO[C@@H](C)c1c(NC(=O)Nc2ccnc(C(F)F)c2)cnc2cc(F)nn12 ; C[C@H](O)c1c(NC(=O)Nc2ccnc(C(F)F)c2)cnc2cc(F)nn12]

Following the procedure in example 39, (S)-1-(2-(difluoromethyl)pyridin-4-yl)-3-(2-fluoro-7-(1-hydroxyethyl) pyrazolo[1,5-a]pyrimidin-6-yl)urea was converted into the title compound. M/z=367 [M+H]+, Rt=0.79 min (UPLC Method B2), ¹H NMR (400 MHz, DMSO-d₆) δ ppm: 10.4 (s, 1H), 8.95 (s, 1H), 8.47 (d, 1H), 7.89 (s, 1H), 7.53 (dd, 1H), 6.89 (t, 1H), 6.52 (d, 1H), 5.68 (q, 1H), 1.50 (d, 3H).

### EXAMPLE 41

### 1-(2-((S)-2-aminopropoxy)-5-chloropyridin-3-yl)-3-(2-chloro-7-((S)-1-methoxyethyl)pvrazolo[1,5-a]pyrimidin-6-yl)urea

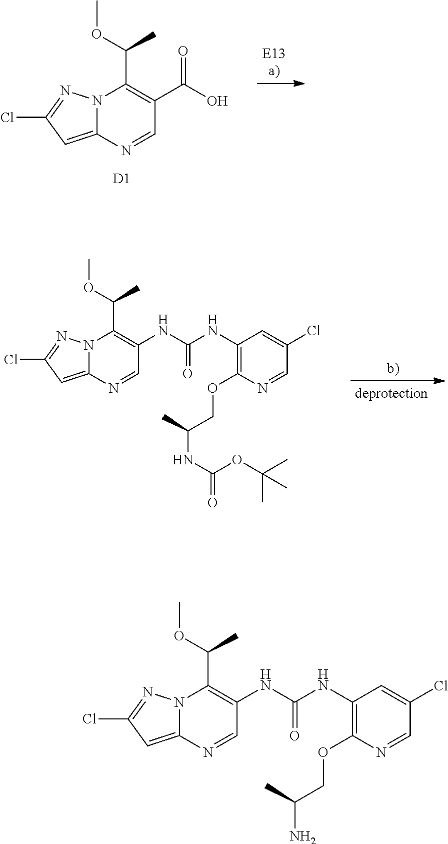

### a) tert-butyl ((S)-1-((5-chloro-3-(3-(2-chloro-7-((S)-1-methoxyethyl)pyrazolo[1,5-a]-pyrimidin-6-yl)ureido)pyridin-2-yl)oxy)propan-2-yl)carbamate

tert-butyl ((S)-1-((5-chloro-3-(3-(2-chloro-7-((S)-1-methoxyethyl)pyrazolo[1,5-a]pyrimidin-6-yl)ureido)pyridin-2-yl)oxy)propan-2-yl)carbamate was prepared analogously as described in example 1 using (S)-tert-butyl (1-((3-amino-5-chloropyridin-2-yl)oxy)propan-2-yl)carbamate instead of (S)-2-chloro-7-(1-methoxyethyl)pyrazolo[1,5-a]pyrimidine-6-carboxylic acid and using (S)-tert-butyl (1-((3-amino-5-chloropyridin-2-yl)oxy)propan-2-yl)carbamate instead of 6-(2H-1,2,3-triazol-2-yl)-5-(trifluoromethyl)pyridin-3-amine. M/z=554-556 [M+H]+, Rt=1.34 min (UPLC Method B2), ¹H NMR (400 MHz, Methanol-d₄) δ ppm: 8.79 (s, 1H), 8.47 (s, 1H), 7.75 (s, 1H), 6.70 (s, 1H), 5.53 (q, 1H), 4.41-4.30 (m, 2H), 4.16 (m, 1H), 3.41 (s, 3H), 1.65 (d, 3H), 1.43 (s, 9H), 1.25 (d, 3H).

### b) 1-(2-((S)-2-aminopropoxy)-5-chloropyridin-3-yl)-3-(2-chloro-7-((S)-1-methoxyethyl)pyrazolo[1,5-a]pyrimidin-6-yl)urea

To tert-butyl ((S)-1-((5-chloro-3-(3-(2-chloro-7-((S)-1-methoxyethyl)pyrazolo[1,5-a]-pyrimidin-6-yl)ureido)pyridin-2-yl)oxy)propan-2-yl)carbamate (56 mg, 0.10 mmol) was added 4N HCl in dioxane (0.5 ml) and the reaction mixture was stirred at RT overnight. The reaction mixture was filtered and the cake dried under HV to afford 1-(2-((S)-2-aminopropoxy)-5-chloropyridin-3-yl)-3-(2-chloro-7-((S)-1-methoxyethyl)-pyrazolo[1,5-a]pyrimidin-6-yl)urea HCl salt. M/z=454-456 [M+H]+, Rt=0.86 min (UPLC Method B2), ¹H NMR (400 MHz, Methanol-d₄) δ ppm: 8.75 (s, 1H), 8.52 (s, 1H), 7.80 (s, 1H), 6.71 (s, 1H), 5.54-5.48 (m, 1H), 4.68-4.59 (m, 1H), 4.53-4.46 (m, 1H), 3.89-3.84 (m, 1H), 3.40 (s, 3H), 1.68 (d, 3H), 1.47 (d, 3H).

### EXAMPLE 42

### (S)-2-(difluoromethyl)-4-(3-(2-fluoro-7-(1-methoxyethyl)pyrazolo[1,5-a]pyrimidin-6-yl)ureido)pyridine 1-oxide

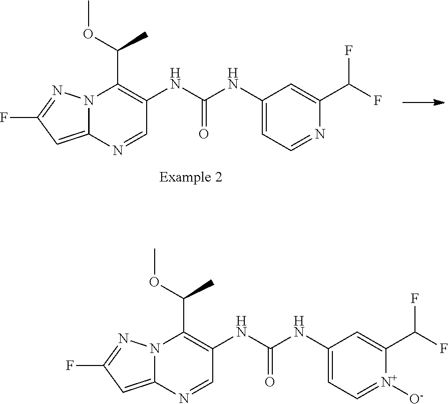 [SMILES: CO[C@@H](C)c1c(NC(=O)Nc2ccnc(C(F)F)c2)cnc2cc(F)nn12 ; CO[C@@H](C)c1c(NC(=O)Nc2cc[n+]([O-])c(C(F)F)c2)cnc2cc(F)nn12]

To a 10 l single-use bioreactor bag was added 1.35 l of thawed cell suspension (E. coli expressing dog cyp d3A12) at OD100, 4.1 l of PSE buffer and 270 ml of Na Citrate 50% (m/m). Finally 504 mg (1.33 mmol) of (S)-1-(2-(difluoromethyl)pyridin-4-yl)-3-(2-fluoro-7-(1-hydroxyethyl) pyrazolo[1,5-a]pyrimidin-6-yl)urea were added. The broth was shaken at 30° C. at 42 rock per minute and an air-flow of 3 l/min. After 23 h, no further progress of the reaction could be detected. The broth was collected, centrifuged, the pellet was extracted 3 times with ACN/MeOH 1/1 and the supernatant was extracted twice with the same volume of ethyl acetate. Organic layers were pooled, concentrated, dried over MgSO4, concentrated and purified by RP-HPLC. Re-purification by SFC (Method A5) provided the title compound. M/z=397 [M+H]+, Rt=0.75 min (UPLC Method B2), ¹H NMR (400 MHz, DMSO-d₆) δ ppm: 10.2 (s, 1H), 8.88 (s, 1H), 8.46 (b rs, 1H), 8.26 (d, 1H), 7.94 (d, 1H), 7.58 (dd, 1H), 7.23 (t, 1H), 6.56 (d, 1H), 5.33 (q, 1H), 1.57 (d, 3H).

### EXAMPLE 43

### (S)-3-chloro-5-(3-(2-fluoro-7-(1-methoxyethyl)pyrazolo[1,5-a]pyrimidin-6-yl)ureido)picolinamide

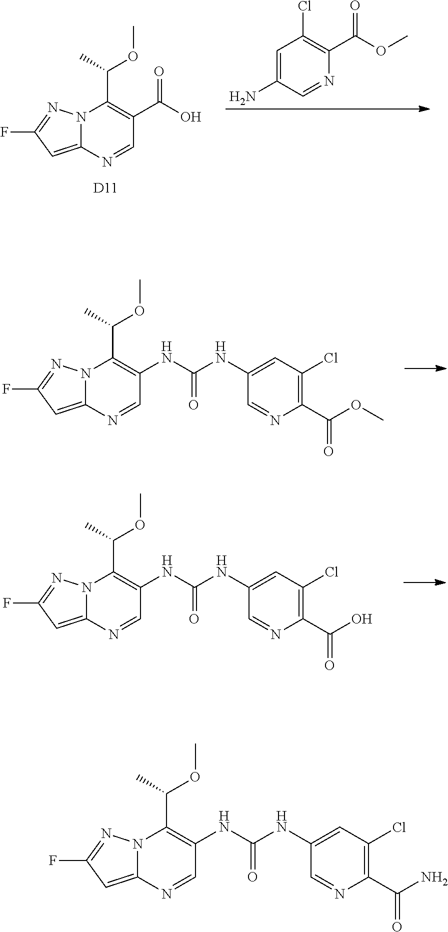

### a) Methyl (S)-3-chloro-5-(3-(2-fluoro-7-(1-methoxyethyl)pyrazolo[1,5-a]pyrimidin-6-yl)ureido)picolinate

To a solution of (S)-2-fluoro-7-(1-methoxyethyl)pyrazolo[1,5-a]pyrimidine-6-carboxylic acid (230 mg, 0.96 mmol) in 1-4-dioxane (1 ml) was added DPPA (0.249 ml, 1.15 mmol) and triethylamine (0.469 ml, 3.37 mmol). The reaction mixture was stirred at RT for 30 mins, then heated to 100° C. for 15 mins. The rection mixture was poured onto sat. aqueous NaHCO3 solution and extracted with ethylacetate. The organic phase was washed with brine, dried over Na2SO4, filtered and the solvent was evaporated. The crude product was purified by Flash-chromatography (ethylacetate/heptane) to yield the title compound. M/z=422 [M+H]+, Rt=0.97 min (UPLC Method B2), ¹H NMR (400 MHz, DMSO-d₆) δ ppm: 10.2 (bs, 1H), 8.88 (s, 1H), 8.56 (d, 1H), 8.53 (bs, 1H), 8.32 (d, 1H), 6.56 (d, 1H), 5.33 (q, 1H), 3.87 (s, 3H), 1.57 (d, 3H), one CH3O signal obscured by solvent peak.

### b) (S)-3-chloro-5-(3-(2-fluoro-7-(1-methoxyethyl) pyrazolo[1,5-a]pyrimidin-6-yl)ureido)picolinic acid

Methyl (S)-3-chloro-5-(3-(2-fluoro-7-(1-methoxyethyl)pyrazolo[1,5-a]pyrimidin-6-yl)ureido)picolinate (200 mg, 0.47 mmol) was suspended in methanol (10 ml). Water (1 ml) and sodium hydroxide (0.47 ml 2N aqeous solution, 0.95 mmol) was added and the reaction mixture was stirred at room temperature for 72 hours. The solvent was evaporated and the residue was acidified with aq. 1N HCl, which led to precipitation of the product. The precipitate was filtered, washed with water and dried. M/z=409 [M+H]+, Rt=0.74 min (UPLC Method B2), ¹H NMR (400 MHz, DMSO-d₆) δ ppm: 13.4 (bs, 1H), 10.16 (s, 1H), 8.89 (s, 1H), 8.56 (d, 1H), 8,53 (s, 1H), 8.28 (d, 1H), 6.56 (d, 1H), 5.34 (q, 1H), 1.58 (d, 3H), CH3O signal obscured by solvent peak.

### c) (S)-3-chloro-5-(3-(2-fluoro-7-(1-methoxyethyl)pyrazolo[1,5-a]pyrimidin-6-yl)ureido)picolinamide

To a solution of (S)-3-chloro-5-(3-(2-fluoro-7-(1-methoxyethyl)pyrazolo[1,5-a]pyrimidin-6-yl)ureido)picolinic acid (100 mg, 0.245 mmol) in DMF (5 ml) at RT was added HATU (112 mg, 0.489 mmol), followed by ammonia in dioxane (0.98 ml, 0.5M solution, 0.489 mmol). The reaction was stirred at RT for 16 hours. Water was added and the mixture was extracted with ethylacetate. The organic phase was washed with water and brine, dried over Na2SO4, filtered and the sovent was evaporated. The crude product was purified by preparative SFC (Method A5) to yield the title compound. M/z=408 [M+H]+, Rt=0.77 min (UPLC Method B2), ¹H NMR (400 MHz, DMSO-d₆) δ ppm: 10.08 (s, 1H), 8.89 (s, 1H), 8.53 (d, 1H), 8.51 (s, 1H), 8.23 (d, 1H), 7.90 (bs, 1H), 7.54 (bs, 1H), 6.56 (d, 1H), 5.35 (q, 1H), 1.57 (d, 3H), CH3O signal obscured by solvent peak.

Biological/Pharmacological Section

The compounds of the invention exhibit valuable pharmacological properties, e.g. properties susceptible to MALT1, for example the inhibition of MALT1 proteolytic and/or autoproteolytic activity e.g. as indicated in the test assays provided infra and are therefore indicated for therapy.

Assays:

MALT1 Biochemical Assay—Version 1:

IC₅₀ values of test compounds, namely examples 1 to 42 and the Reference compound, were determined with an enzyme activity assay using the C-domain of MALT1 (amino acids 329-824). The readout parameter is the increase of fluorescence lifetime over time, proportional to enzyme activity.

The assay employs a short peptide substrate labeled with the single fluorophore PT14 as a fluorescence lifetime probe sensitive to the cleavage state of the substrate (PT14: 6-(9-oxo-9H-acridin-10-yl)-hexanoate, AssayMetrics, UK). The peptide substrate has the following sequence: Ac-Trp-Leu-Arg-Ser-Arg\hat{ }Cys(PT14)-NH₂ (Product number BS-9117, Biosyntan, Germany, N-terminus to C-terminus from left to right in three letter code, Ac: acetyl group, Cys(PT14): cysteine residue with the fluorophore PT14 conjugated to the cysteine sulfhydryl group via a maleimide group; C-terminus of the peptide is amidated; within the substrate sequence written above, A indicates the scissile bond). The assay buffer consists of 200 mM Tris/HCl at pH 7.5, 0.8 M Na citrate, 100 μM EGTA, 100 μM DTT and 0.05% (w/v) CHAPS. The kinetic characterization of the enzymatic reaction led to the determination of a Michaelis Constant (K_{M}) of 40 μM and a kcat value of 34 s⁻¹. The assay was established for the 384-well plate format using black microtiter round well plates (Product number 95040020, Thermo Electron Oy, Finland). Test compounds were dissolved in 100% (v/v) DMSO or a mixture containing 90% (v/v) DMSO and 10% (v/v) H₂O at a stock concentration of 100 mM. Serial dilutions of test compounds were prepared using either 100% (v/v) DMSO or a mixture containing 90% (v/v) DMSO and 10% (v/v) H₂O.

For the measurement of compound inhibition, 0.25 μl of test compound were mixed with 12.5 μl of enzyme in wells of the 384-well plates, and incubated for 60 minutes at room temperature (22° C.). After that, 12.5 μl of substrate was added, and the enzymatic reaction was allowed to proceed for 60 minutes at room temperature (22° C.). The total assay volume was 25.25 μl, and the final assay concentrations for enzyme and substrate were 2.5 nM and 1 μM, respectively. The increase in assay signal over time is linear for at least 60 minutes at the assay conditions reported, and directly proportional to the concentration of active enzyme up to at least 2.5 nM. The DMSO content was between 0.9 and 1% (v/v). The final assay concentrations of the test compounds ranged typically from 100 μM to 1 nM in a serial dilution series using a dilution factor of 3.16 (i.e. half-logarithmic dilution steps). As controls, reactions were performed in multiple wells either by only adding DMSO instead of test compound, leading to an uninhibited enzymatic reaction (i.e. 0% inhibition), or by adding assay buffer without enzyme mixed with DMSO, which is the equivalent of a fully inhibited reaction (i.e. 100% inhibition). The fluorescence lifetimes were recorded using a microtiter plate reader such as the TECAN Ultra Evolution FLT instrument with fluorescence excitation at 405 nm and emission recording at 450 nm. The fluorescence lifetimes can be transformed to percentage inhibitions using the above mentioned controls as reference (for 0 and 100% inhibition). The IC₅₀ value was calculated from the plot of percentage inhibition versus inhibitor concentration using non-linear regression analysis software (Origin, OriginLab Corporation, USA). The data were fitted using a 4 Parameter Logistic Model, characterized by the following equation:

y=A2(A1−A2)/(1+(x/IC50)\hat{ }p)

where y is the %-inhibition at the inhibitor concentration, x. A1 is the lowest inhibition value, and A2 the maximum inhibition value. The exponent, p, is the Hill coefficient.

Alternatively the biochemical activity of some examples, namely example 43 and the Reference compound, were determined by measuring fluorescence intensity as described below:

MALT1 Biochemical Assay—Version 2:

IC₅₀ values of test compounds were determined with an enzyme activity assay using the C-domain of MALT1 (amino acids 329-824). The readout parameter is the increase of fluorescence intensity, proportional to enzyme activity.

The assay employs a short peptide substrate labeled with the fluorophore Rhodamine 110 (Rh110) as a fluorescence probe sensitive to the cleavage state of the substrate. The peptide substrate has the following sequence: Ac-Leu-Arg-Ser-Arg\hat{ }Rh110-dPro (Product number BS-3027, Biosyntan, Germany; within the substrate sequence, A indicates the scissile bond). The assay buffer consists of 200 mM Tris/HCl at pH 7.5, 0.8 M Na citrate, 100 μM EGTA, 100 μM DTT and 0.05% (w/v) CHAPS. The kinetic characterization of the enzymatic reaction led to the determination of a Michaelis Constant (K_{M}) of 40 μM and a kcat value of 34 s⁻¹. The assay was established for the 384-well plate format using black microtiter well plates. Test compounds were dissolved in 100% (v/v) DMSO or a mixture containing 90% (v/v) DMSO and 10% (v/v) H₂O at a stock concentration of 100 mM. Serial dilutions of test compounds were prepared using either 100% (v/v) DMSO or a mixture containing 90% (v/v) DMSO and 10% (v/v) H₂O.

For the measurement of compound inhibition, 0.1 μl of test compound were mixed with 5 μI of enzyme in wells of the 384-well plates, and incubated for 60 minutes at room temperature (22° C.). After that, 5μl of substrate was added, and the enzymatic reaction was allowed to proceed for 60 minutes at room temperature (22° C.). The total assay volume was 10 μl, and the final assay concentrations for enzyme and substrate were 2 nM and 1 μM, respectively. The DMSO content was between 0.9 and 1% (v/v). The final assay concentrations of the test compounds ranged typically from 100 μM to 0.007 nM in a serial dilution series using a dilution factor of 3.16 (i.e. half-logarithmic dilution steps). As controls, reactions were performed in multiple wells either by only adding DMSO instead of test compound, leading to an uninhibited enzymatic reaction (i.e. 0% inhibition), or by adding assay buffer without enzyme mixed with DMSO, which is the equivalent of a fully inhibited reaction (i.e. 100% inhibition). The fluorescence intensities were recorded using a microtiter plate reader such as the Wallac EnVision instrument (Perkin Elmer) with fluorescence excitation at 485 nm and emission recording at 535 nm. The IC₅₀ value was calculated from the plot of percentage inhibition versus inhibitor concentration using non-linear regression analysis software (Origin, OriginLab Corporation, USA). The data were fitted using a 4 Parameter Logistic Model, characterized by the following equation:

y=A2+(A1−A2)/(1+(x/IC50)\hat{ } p) where y is the %-inhibition at the inhibitor concentration, x. A1 is the lowest inhibition value, and A2 the maximum inhibition value. The exponent, p, is the Hill coefficient.

Human IL2 Promoter Reporter Gene Assay (RGA) in Jurkat Cells

The transfected Jurkat clone K22 290_H23 was propagated in RPMI 1640 supplemented with 10% heat inactivated fetal calf serum, 50 μM 2-mercaptoethanol and 1 mg/ml Geneticin. The cell concentration should not exceed 1×10e6/ml during culturing. The cells should not exceed passage 30. Prior to the assay the cells were washed and prepared to the concentration of 2×10e6 cells/ml.

Compound dilutions were made as 2×-concentrated solutions then diluted ½ by addition to cells. Two hundred and fifty μl of compound dilution and 250 μl of cells were mixed together in wells of a 96-deep well plate. Cells/compounds premix were incubated 30 min at 37° C. and 5% CO2 directly in the deep well plate.

After pre-incubation of cells with compounds, cells were stimulated with anti-CD28 mAb (clone 15E8) at 3 μg/ml+PMA at 1 μg/ml. Both co-stimulants were diluted in culture medium at a 10×-concentrated solution. 10 μl of co-stimulants were pipetted into the white 96-well plates and 100 μl of cell/compound mix was immediately added in duplicates. The cells were stimulated for 5.5 h at 37° C. and 5% CO2.

After cell stimulation, 50 μl of BriteLitePlus reagent (Perkin Elmer) was added to each well and the bioluminescence was measured with a Wallac EnVision reader (Perkin Elmer).

Using the assays described above the following IC₅₀s were determined:

| | MALT1 | MALT1 | IL2 |
|---|---|---|---|
| | biochemical | biochemical | reporter |
| | activity - | activity - | gene |
| | Version 1 - | Version 2 - | assay - |
| Example | IC₅₀ (nM) | IC₅₀ (nM) | IC₅₀ (nM) |
| 1 | 92 | | 133 |
| 2 | 18 | | 47 |
| 3 | 4 | | 36 |
| 4 | 3 | | 103 |
| 5 | 2 | | 19 |
| 6 | 4 | | 41 |
| 7 | 7 | | 27 |
| 8 | 2 | | 38 |
| 9 | 29 | | 93 |
| 10 | 3 | | 22 |
| 11 | 2 | | 12 |
| 12 | 31 | | 73 |
| 13 | 23 | | 88 |
| 14 | 3 | | 6 |
| 15 | 9 | | 12 |
| 16 | 3 | | 20 |
| 17 | 20 | | 87 |
| 18 | 4 | | 19 |
| 19 | 9 | | 56 |
| 20 | 9 | | 101 |
| 21 | 9 | | 47 |
| 22 | 7 | | 25 |
| 23 | 16 | | 42 |
| 24 | 14 | | 62 |
| 25 | 4 | | 21 |
| 26 | 22 | | 113 |
| 27 | 8 | | 35 |
| 28 | 13 | | 45 |
| 29 | 50 | | 135 |
| 30 | 7 | | 20 |
| 31 | 12 | | 35 |
| 32 | 11 | | 40 |
| 33 | 11 | | 44 |
| 34 | 7 | | 28 |
| 35 | 10 | | 17 |
| 36 | 3 | | 35 |
| 37 | 25 | | 57 |
| 38 | 24 | | 86 |
| 39 | 72 | | 604 |
| 40 | 11 | | 100 |
| 41 | 21 | | 50 |
| 42 | 96 | | 729 |
| 43 | | 8 | 69 |
| Reference** | 12 | 6 | |
| compound for | | | |
| calibrating the | | | |
| MALT1 assays | | | |
| Version 1 and 2 | | | |

Reference** compound taken above is described in WO 93/09135 (example No. 49), the structure of which is displayed below:

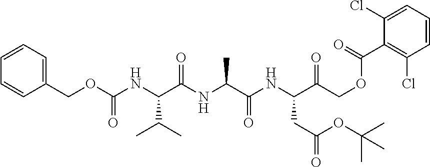 [SMILES: CC(C)[C@H](NC(=O)OCc1ccccc1)C(=O)N[C@@H](C)C(=O)N[C@@H](CC(=O)OC(C)(C)C)C(=O)COC(=O)c1c(Cl)cccc1Cl]

Utilities

According to the results obtained in the test assays provided above, it is contemplated that the compounds of the invention may be useful in the treatment of a disease or disorder (an indication) selected from:

Conditions and disorders characterized by disregulated NF-kB activation, in particular autoimmune/immunological and inflammatory disorders, allergic disorders, respiratory disorders and oncological disorders.

Said autoimmune and inflammatory disorders may inter alia be selected from arthritis, ankylosing spondylitis, inflammatory bowel disease, ulcerative colitis, gastritis, pancreatitis, Crohn's disease, celiac disease, multiple sclerosis, systemic lupus erythematosus, rheumatoid arthritis, rheumatic fever, gout, organ or transplant rejection, acute or chronic graft-versus-host disease, chronic allograft rejection, Behcet's disease, uveitis, psoriasis, dermatitis, atopic dermatitis, dermatomyositis, myasthena gravis, Grave's disease, Hashimoto thyroiditis, Sjögren's syndrome, and blistering disorders (e.g. pemphigus vulgaris), antibody-mediated vasculitis syndromes, including ANCA-associated vasculitides, Hennoch-Schönlein Purpura, and immune-complex vasculitides (either primary or secondary to infection or cancers).

Said oncological disorders may inter alia be selected from carcinoma, sarcoma, lymphoma, leukemia and germ cell tumors, e.g. adenocarcinoma , bladder cancer, clear cell carcinoma, skin cancer, brain cancer, cervical cancer, colon cancer, colorectal cancer, endometrial cancer, bladder cancer, brain tumours, breast cancer, gastric cancer, germ cell tumours, glioblastoma, hepatic adenomas, Hodgkin's lymphoma, liver cancer, kidney cancer, lung cancer, ovarian cancer, dermal tumours, prostate cancer, renal cell carcinoma, stomach cancer, medulloblastoma, non-Hodgkin's lymphoma, diffuse large B-cell lymphoma, mantle cell lymphoma, marginal zone lymphoma,T cell lymphomas, in particular Sézary syndrome, Mycosis fungoides, cutaneous T-cell lymphoma, T-cell acute lymphoblastic leukemia, melanoma , mucosa-associated lymphoid tissue (MALT) lymphoma, multiple myeloma, plasma cell neoplasm, lentigo maligna melanomas, acral lentiginous melanoma, and squamous cell carcinoma.

Said allergic disorder may inter alia be selected from contact dermatitis, celiac disease, asthma, hypersensitivity to house dust mites, pollen and related allergens, Berylliosis Said respiratory disorders may inter alia be selected from asthma, bronchitis, chronic obstructive pulmonary disease (COPD), cystic fibrosis, pulmonary oedema, pulmonary embolism, pneumonia, pulmonary sarcoidosis, silicosis, pulmonary fibrosis, respiratory failure, acute respiratory distress syndrome, primary pulmonary hypertension and emphysema.

In another embodiment the compounds of the invention may be useful in the treatment of rheumatoid arthritis, systemic lupus erythematosus, vasculitic conditions, allergic diseases, asthma, chronic obstructive pulmonary disease (COPD), acute or chronic transplant rejection, graft versus host disease, cancers of hematopoietic origin or solid tumors, chronic myelogenous leukemia, myeloid leukemia, non-Hodgkin lymphoma or other B cell lymphomas.

In another embodiment the compounds of the invention may be useful in the treatment of BENTA disease, berylliosis, rheumatoid arthritis, systemic lupus erythematosus, lupus nephritis, multiple sclerosis, polymyositis, psoriasis, ABC-DLBCL, e.g. with activating mutations in Card11, MALT lymphomas.

In addition to the named conditions and disorders characterized by disregulated NF-kB, conditions and disorders characterized by dysregulated IL-17 secretion—in addition to or independent of dysregulated NF-kB—include psoriasis, psoriatic arthritis, acne vulgaris, hidradenitis suppurativa, atopic dermatitis.

Combinations

The compound of the present invention may be administered either simultaneously with, or before or after, one or more other therapeutic agent. The compound of the present invention may be administered separately, by the same or different route of administration, or together in the same pharmaceutical composition as the other agents.

The compounds of the invention may be administered as the sole active ingredient or in conjunction with, e.g. as an adjuvant to, other drugs e.g. immunosuppressive or immunomodulating agents or other anti-inflammatory agents, e.g. for the treatment or prevention of allo- or xenograft acute or chronic rejection or inflammatory or autoimmune disorders, or a chemotherapeutic agent, e.g a malignant cell anti-proliferative agent.

For example, the compounds of the invention may be used in combination with a calcineurin inhibitor, e.g. cyclosporin A or FK 506; a mTOR inhibitor, e.g. rapamycin, 40-O-(2-hydroxyethyl)-rapamycin, biolimus-7 or biolimus-9; an ascomycin having immunosuppressive properties, e.g. ABT-281, ASM981; corticosteroids; cyclophosphamide; azathioprene; methotrexate; leflunomide; mizoribine; mycophenolic acid or salt; mycophenolate mofetil; IL-1beta inhibitor.

In another embodiment compounds of the invention are combined with a co-agent which are PI3Kinase inhibitors.

In another embodiment compounds of the invention are combined with co-agent that influence BTK (Bruton's tyrosine kinase).

For the treatment of oncological diseases compounds of the invention may be used in combination with B-cell modulating agents, e.g. Rituximab, Ofatumumab, Btk or Syk inhibitors, inhibitors of PKC, PI3 kinases, PDK, PIM, JAK and mTOR and BH3 mimetics.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of formula (I) and a co-agent, are both administered to a patient simultaneously in the form of a single entity or dosage. The term “non-fixed combination” means that the active ingredients, e.g. a compound of formula (I) and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

In one embodiment, the invention provides a product comprising a compound of formula (I) and at least one other therapeutic agent as a combined preparation for simultaneous, separate or sequential use in therapy. In one embodiment, the therapy is the treatment of a disease or condition mediated by MALT1. Products provided as a combined preparation include a composition comprising the compound of formula (I) and the other therapeutic agent(s) together in the same pharmaceutical composition, or the compound of formula (I) and the other therapeutic agent(s) in separate form, e.g. in the form of a kit.

In one embodiment, the invention provides a pharmaceutical composition comprising a compound of formula (I) and another therapeutic agent(s). Optionally, the pharmaceutical composition may comprise a pharmaceutically acceptable excipient, as described above.

In one embodiment, the invention provides a kit comprising two or more separate pharmaceutical compositions, at least one of which contains a compound of formula (I). In one embodiment, the kit comprises means for separately retaining said compositions, such as a container, divided bottle, or divided foil packet. An example of such a kit is a blister pack, as typically used for the packaging of tablets, capsules and the like.

The kit of the invention may be used for administering different dosage forms, for example, oral and parenteral, for administering the separate compositions at different dosage intervals, or for titrating the separate compositions against one another. To assist compliance, the kit of the invention typically comprises directions for administration.

1. A compound of formula (I) or a pharmaceutically acceptable salt thereof, wherein 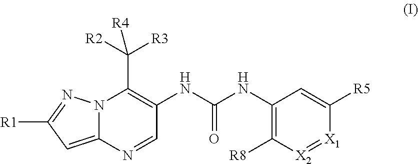
R1 is fluoro, chloro, methyl or cyano;
R2 and R 3 are independently from each other C₁-C₆ alkoxy optionally substituted by C₁-C₆ alkoxy; C₁-C₆ alkyl optionally substituted by halogen or C₁-C₆ alkoxy; amino optionally substituted by C₁-C₆ alkyl ; phthalimido; or hydroxy optionally substituted by a 5 or 6 membered heterocyclic ring comprising a nitrogen or oxygen heteroatom wherein said ring is optionally substituted by C₁-C₃ alkyl carbonyl;
or R₂ and R₃ together with carbon atom to which they are attached form a 3-5 membered carbocyclic ring or heterocyclic ring comprising 1 heteroatom selected from N and O;
R4 is hydrogen; C₁-C₆ alkyl optionally substituted by C₁-C₆ alkoxy;
X1 is N, N—O or CR6;
X2 is N or CR7;
R5 is chloro; cyano; or C₁-C₆ alkyl optionally substituted by halogen and/or hydroxy;
R6 is hydrogen; oxo; methoxy; 1,2,3-triazole-2-yl; or aminocarbonyl substituted at the nitrogen atom by R9 and R10;
R7 is hydrogen; C₁-C₆ alkyl optionally substituted by halogen and/or hydroxy; or N,N-dimethylaminocarbonyl;
R8 is hydrogen; C₁-C₆ alkoxy optionally substituted by methoxy or amino;
R9 and 10 are independently of each other hydrogen; C₁-C₆ alkyl optionally substituted by C₁-C₆ alkoxy, N-mono-C₁-C₆ alkyl amino, or N, N-di-C₁-C₆ alkyl amino; or
R9 and 10 together with the nitrogen atom to which they are attached form a 5-7 membered heterocyclic ring having one, two or three ring hetero atoms selected from the group consisting of oxygen, nitrogen and sulphur, that ring being optionally substituted by C₁-C₆ alkyl, hydroxy or oxo;
with the proviso that X1 and X2 must not be N at the same time, or Xi must not be N—O when X2 is N.

2. A compound of claim 1 or a pharmaceutically acceptable salt thereof, wherein
R1 is fluoro or chloro;
R2 is C₁-C₆ alkyl optionally substituted by C₁-C₆ alkoxy;
R3 is C₁-C₆ alkoxy optionally be substituted by C₁-C₆ alkoxy;
R4 is hydrogen;
X₁ is N;
X₂ is CR7;
R5 is chloro; cyano; difluoromethyl; trifluoromethyl;
R7 is hydrogen; and
R8 is hydrogen.

3. A compound of claim 1 or a pharmaceutically acceptable salt thereof, wherein
R1 is fluoro or chloro;
R2 is C₁-C₆ alkyl optionally substituted by C₁-C₆ alkoxy;
R3 is C₁-C₆ alkoxy optionally be substituted by C₁-C₆ alkoxy;
R4 is hydrogen;
X₁ is CR6;
X₂ is N;
R5 is chloro; cyano; difluoromethyl; trifluoromethyl;
R6 is hydrogen; oxo; methoxy; 1,2,3-triazole-2-yl; N,N-dimethylaminocarbonyl; pyrrolidin-1-yl carbonyl and
R8 is hydrogen.

4. A compound of claim 1 or a pharmaceutically acceptable salt thereof, wherein
R1 is methyl, fluoro or chloro;
R2 is C₁-C₆ alkyl;
R3 is C₁-C₆ alkoxy;
R4 is hydrogen;
X₁ is CR6;
X₂ is N;
R5 is chloro; cyano; difluoromethyl; trifluoromethyl;
R6 is hydrogen; methoxy; 1,2,3-triazole-2-yl; N,N-dimethylamino carbonyl; pyrrolidin-1-yl carbonyl and
R8 is hydrogen.

5. A compound of claim 1 or a pharmaceutically acceptable salt thereof, wherein
R1 is methyl, fluoro or chloro;
R2 is C₁-C₆ alkyl;
R3 is C₁-C₆ alkoxy;
R4 is hydrogen;
X₁ is N;
X₂ is CR7;
R5 is chloro; cyano; difluoromethyl; trifluoromethyl;
R7 is hydrogen; and
R8 is hydrogen.

6. A compound of claim 1 or a pharmaceutically acceptable salt thereof, wherein
R1 is fluoro or chloro;
R2 is C₁-C₆ alkoxy;
R3 is C₁-C₆ alkyl;
R4 is hydrogen;
X₁ is CR6;
X₂ is N;
R5 is chloro; cyano; difluoromethyl; trifluoromethyl;
R6 is hydrogen; methoxy; 1,2,3-triazole-2-yl; N,N-dimethylamino carbonyl; pyrrolidin-1-yl carbonyl and
R8 is hydrogen.

7. A compound of claim 1 or a pharmaceutically acceptable salt thereof, wherein
R1 is fluoro or chloro;
R2 is C₁-C₆ alkoxy;
R3 is C₁-C₆ alkyl;
R4 is hydrogen;
X₁ is N;
X₂ is CR7;
R5 is chloro; cyano; difluoromethyl; trifluoromethyl;
R7 is hydrogen; and
R8 is hydrogen.

8. A compound, in particular of claim 1 or a pharmaceutically acceptable salt thereof, wherein the compound is selected from
(S)-1-(5-cyanopyridin-3-yl)-3-(7-(1-methoxyethyl)-2-methylpyrazolo [1,5-a]pyrimidin-6-yl)urea;
(S)-1-(2-(difluoromethyl)pyridin-4-yl)-3-(2-fluoro-7-(1-methoxyethyl) pyrazolo[1,5-a]pyrimidin-6-yl)urea;
1-(5-chloro-6-(2H-1,2,3-triazol-2-yl)pyridin-3-yl)-3-(2-chloro-7-isopropylpyrazolo[1,5-a]pyrimidin-6-yl)urea;
(S)-1-(5-chloro-6-(2H-1,2,3-triazol-2-yl)pyridin-3-yl)-3-(2-chloro-7-(1-methoxyethyl)pyrazolo[1,5-a]pyrimidin-6-yl)urea;
(S)-1-(5-cyano-6-methoxypyridin-3-yl)-3-(2-fluoro-7-(1-methoxyethyl) pyrazolo[1,5-a]pyrimidin-6-yl)urea;
(S)-1-(6-(2H-1,2,3-triazol-2-yl)-5-(trifluoromethyl)pyridin-3-yl)-3-(2-chloro-7-(1-(2-methoxyethoxy) ethyl)pyrazolo[1,5-a]pyrimidin-6-yl)urea;
(S)-1-(6-(2H-1,2,3-triazol-2-yl)-5-(trifluoromethyl)pyridin-3-yl)-3-(2-chloro-7-(1-methoxy-2-methyl-propyl)pyrazolo[1,5-a]pyrimidin-6-yl)urea;
1-(2-chloro-7-(1-(methoxymethyl)cyclopropyl)pyrazolo[1,5-a]pyrimidin-6-yl)-3-(5-cyanopyridin-3-yl)urea;
1-(5-chloro-6-(2H-1,2,3-triazol-2-yl)pyridin-3-yl)-3-(2-chloro-7-((1R,2S)-1,2-dimethoxypropyl) pyrazolo[1,5-a]pyrimidin-6-yl)urea;
(S)-1-(2-chloro-7-(1-methoxyethyl)pyrazolo[1,5-a]pyrimidin-6-yl)-3-(5-cyano-6-(2H-1,2,3-triazol-2-yl)pyridin-3-yl)urea;
(S)-1-(5-cyanopyridin-3-yl)-3-(2-fluoro-7-(1-methoxyethyl)pyrazolo[1,5-a]pyrimidin-6-yl)urea;
1-(7-((S)-1-(((R)-1-acetylpyrrolidin-3-yl)oxy)ethyl)-2-chloropyrazolo[1,5-a]pyrimidin-6-yl)-3-(5-chloro-6-(2H-1,2,3-triazol-2-yl)pyridin-3-yl)urea; (S)-1-(5-chloro-6-(2H-1,2,3-triazol-2-yl)pyridin-3-yl)-3-(2-fluoro-7-(1-methoxy-2-methylpropyl)-pyrazolo[1,5-a]pyrimidin-6-yl)urea;
(8)-1-(5-cyano-6-(2H-1,2,3-triazol-2-yl)pyridin-3-yl)-3-(7-(1-methoxy-2-methylpropyl)-2-methylpyrazolo[1,5-a]pyrimidin-6-yl)urea;
(S)-1-(2-chloro-7-(1-methoxyethyl)pyrazolo[1,5-a]pyrimidin-6-yl)-3-(5-cyano-6-methoxypyridin-3-yl)urea;
1-(2-fluoro-7-((S)-1-methoxyethyl)pyrazolo[1,5-a]pyrimidin-6-yl)-3-(2-(1-hydroxyethyl)-6-(trifluoromethyl)pyridin-4-yl)urea;
(S)-1-(5-cyano-6-(2H-1,2,3-triazol-2-yl)pyridin-3-yl)-3-(2-fluoro-7-(1-methoxyethyl)pyrazolo[1,5-a]pyrimidin-6-yl)urea;
1-(2-chloro-7-(1,2-dimethoxyethyl)pyrazolo[1,5-a]pyrimidin-6-yl)-3-(5-cyano-6-(2H-1,2,3-triazol-2-yl)pyridin-3-yl)urea;
1-(2-chloro-7-((S)-1-methoxyethyl)pyrazolo[1,5-a]pyrimidin-6-yl)-3-(2-(2,2,2-trifluoro-1-hydroxy-ethyl)pyridin-4-yl)urea;
(S)-1-(5-chloro-2-(2-methoxyethoxy)pyridin-3-yl)-3-(2-chloro-7-(1-methoxyethyl)-pyrazolo[1,5-a]-pyrimidin-6-yl)urea;
(S)-1-(5-cyano-6-methoxypyridin-3-yl)-3-(7-(1-methoxy-2-methylpropyl)-2-methylpyrazolo[1,5-a]-pyrimidin-6-yl)urea;
(S)-1-(2-cyanopyridin-4-yl)-3-(2-fluoro-7-(1-methoxyethyl)pyrazolo[1,5-a]pyrimidin-6-yl)urea; (S)-1-(5-cyano-6-methoxypyridin-3-yl)-3-(7-(1-methoxyethyl)-2-methylpyrazolo[1,5-a]pyrimidin-6-yl)urea;
1-(2-chloro-7-((1R ,2S)-1 ,2-dimethoxypropyl)pyrazolo[1,5-a]pyrimidin-6-yl)-3-(5-cyano-6-(2H-1,2,3-triazol-2-yl)pyridin-3-yl)urea;
1-(7-((S)-1-(((S)-1-acetylpyrrolidin-3-yl)oxy)ethyl)-2-chloropyrazolo[1,5-a]pyrimidin-6-yl)-3-(5-cyano-6-methoxypyridin-3-yl)urea;
(S)-1-(5-cyano-6-(2H-1,2,3-triazol-2-yl)pyridin-3-yl)-3-(7-(1-methoxyethyl)-2-methylpyrazolo[1,5-a]pyrimidin-6-yl)urea;
(S)-6-chloro-4-(3-(2-chloro-7-(1-methoxyethyl)pyrazolo[1,5-a]pyrimidin-6-yl)ureido)-N,N-dimethylpicolinamide;
(S)-1-(5-(difluoro-methyl)pyridin-3-yl)-3-(2-fluoro-7-(1-methoxyethyl)-pyrazolo[1,5-a]pyrimidin-6-yl)urea;
(S)-1-(2-fluoro-7-(1-methoxyethyl)pyrazolo[1,5-a]pyrimidin-6-yl)-3-(5-(trifluoro-methyl)pyridin-3-yl)urea;
(S)-3-chloro-5-(3-(2-chloro-7-(1-methoxyethyl)pyrazolo[1,5-a]pyrimidin-6-yl)ureido)-N,N-dimethylpicolinamide;
(S)-1-(5-chloro-pyridin-3-yl)-3-(2-fluoro-7-(1-methoxyethyl)pyrazolo[1,5-a]pyrimidin-6-yl)urea;
(S)-1-(5-chloro-6-(pyrrolidine-1-carbonyl)pyridin-3-yl)-3-(2-chloro-7-(1-methoxyethyl)pyrazolo-[1,5-a]pyrimidin-6-yl)urea
(S)-3-chloro-5-(3-(2-chloro-7-(1-methoxyethyl)pyrazolo[1,5-a]pyrimidin-6-yl)ureido)-N-methylpicolinamide
(S)-1-(2-chloro-7-(1-methoxyethyl)pyrazolo[1,5-a]pyrimidin-6-yl)-3-(5-chloropyridin-3-yl)urea;
(S)-1-(7-(1-aminoethyl)-2-chloropyrazolo[1,5-a]pyrimidin-6-yl)-3-(5-chloro-6-(2H-1,2,3-triazol-2-yl)pyridin-3-yl)urea;
(S)-1-(5-cyanopyridin-3-yl)-3-(7-(1-hydroxyethyl)-2-methylpyrazolo[1,5-a]pyrimidin-6-yl)urea;
(S)-1-(2-(difluoromethyl)pyridin-4-yl)-3-(2-fluoro-7-(1-hydroxyethyl) pyrazolo[1,5-a]pyrimidin-6-yl)urea;
1-(2-((S)-2-aminopropoxy)-5-chloropyridin-3-yl)-3-(2-chloro-7-((S)-1-methoxyethyl)pyrazolo[1,5-a]pyrimidin-6-yl)urea;
(S)-2-(difluoromethyl)-4-(3-(2-fluoro-7-(1-methoxyethyl)pyrazolo[1,5-a]pyrimidin-6-yl)ureido)pyridine 1-oxide;
1-(2-chloro-7-((1R,2S)-1,2-dimethoxypropyl)pyrazolo[1,5-a]pyrimidin-6-yl)-3-(5-cyano-6-methoxypyridin-3-yl)urea;
1-(2-chloro-7-(1-(methoxymethyl)cyclopropyl)pyrazolo[1,5-a]pyrimidin-6-yl)-3-(2-cyanopyridin-4-yl)urea; and
(S)-3-chloro-5-(3-(2-fluoro-7-(1-methoxyethyl)pyrazolo[1,5-a]pyrimidin-6-yl)ureido)picolinamide.

9. A pharmaceutical composition comprising a therapeutically effective amount of a compound according to claim 1 or a pharmaceutically acceptable salt thereof and one or more pharmaceutically acceptable carriers.

10. A combination comprising a therapeutically effective amount of a compound according to claim 1 or a pharmaceutically acceptable salt thereof and one or more therapeutically active co-agents.

11. A method of modulating MALT1 activity in a subject, wherein the method comprises administering to the subject a therapeutically effective amount of a compound according to claim 1 or a pharmaceutically acceptable salt thereof.

12. (canceled)

13. A compound according to claim 1 being a compound of formula (II) or a pharmaceutically acceptable salt thereof, wherein 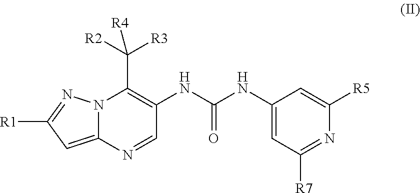
R1 is fluoro or chloro;
R2 and R3 are independently from each other C₁-C₆ alkyl or C₁-C₆ alkoxy;
R4 is hydrogen;
R5 and R7 are independently from each other hydrogen; cyano; halogen or C₁-C₆ alkyl optionally substituted by fluoro and/or hydroxyl.

14. A compound according to claim 1 being a compound of formula (III) or a pharmaceutically acceptable salt thereof, wherein 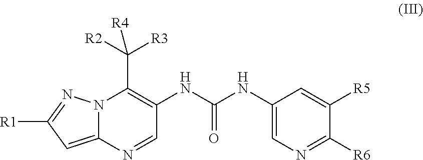
R1 is fluoro or chloro;
R2 and R3 are independently from each other C₁-C₆ alkyl or C₁-C₆ alkoxy;
R4 is hydrogen;
R5 is hydrogen; cyano; halogen or C₁-C₆ alkyl optionally substituted by fluoro and/or hydroxyl; and
R6 is hydrogen; 1,2,3-triazole-2-yl; N,N-dimethylaminocarbonyl; N-monomethylamino carbonyl; or pyrrolidin-1-yl carbonyl.

15. A compound of claim 1 or a pharmaceutically acceptable salt thereof, wherein X₁ is N and X₂ is not N, or X, is not N and X₂ is N.

16. A pharmaceutical combination comprising a compound according to claim 1, or a pharmaceutically acceptable salt thereof, and
(a) immunosuppressive agents, immunomodulating agents, or a chemotherapeutic agent
(b) a calcineurin inhibitor, e.g. cyclosporin A or FK 506; a mTOR inhibitor, e.g. rapamycin, 40-O-(2-hydroxyethyl)-rapamycin, biolimus-7 or biolimus-9; an ascomycin having immunosuppressive properties, e.q. ABT-281, ASM981; corticosteroids; cyclophosphamide; azathioprene; methotrexate; leflunomide: mizoribine; mycophenolic acid or salt; mycophenolate mofetil; IL-1beta inhibitor; or
(c) a coagent which are PI3Kinase inhibitors; or
(d) a coagent which influence BTK (Bruton's tyrosine kinase); or
(e) B-cell modulating agents, e.q. Rituximab, Ofatumumab, Btk or Syk inhibitors, inhibitors of PKC, PI3 kinases, PDK, PIM. JAK and mTOR and BH3 mimetics.

17. A crystalline form which is selected from:
(a) a crystalline form of (S)-1-(5-cyanopyridin-3-yl)-3-(7-(1-methoxyethyl)-2-methylpyrazolo [1,5-a]pyrimidin-6-yl)urea;
(b) a crystalline hydrate form of (S)-1-(2-(difluoromethyl)pyridin-4-yl)-3-(2-fluoro-7-(1-methoxyethyl) pyrazolo[1,5-a]pyrimidin-6-yl)urea;
(c) a crystalline hemi-hydrate form of (S)-1-(2-chloro-7-(1-methoxyethyl)pyrazolo[1,5-a]pyrimidin-6-yl)-3-(2-(trifluoromethyl)pyridin-4-yl)urea;
(d) a crystalline from of 1-(5-chloro-6-(2H-1,2,3-triazol-2-yl)pyridin-3-yl)-3-(2-chloro-7-isopropylpyrazolo[1,5-a]pyrimidin-6-yl)urea; and
(e) a crystalline form of (S)-1-(5-chloro-6-(2H-1,2,3-triazol-2-yl)pyridin-3-yl)-3-(2-chloro-7-(1-methoxyethyl)pyrazolo[1,5-a]pyrimidin-6-yl)urea.

18. A crystalline form of (S)-1-(5-cyanopyridin-3-yl)-3-(7-(1-methoxyethyl)-2-methylpyrazolo [1,5-a]pyrimidin-6-yl)urea according to claim 17, characterized by an X-ray powder diffraction pattern with peaks at 11.36°±0.2°, and 25.49±0.2°.

19. A crystalline hydrate form of (S)-1-(2-(difluoromethyl)pyridin-4-yl)-3-(2-fluoro-7-(1-methoxyethyl) pyrazolo[1,5-a]pyrimidin-6-yl)urea according to claim 17, characterized by an X-ray powder diffraction pattern with peaks at 8.58°±0.2°, 11.21°±0.2°, 19.67±0.2°, and 22.01±0.2°.

20. A crystalline hemi-hydrate form of (S)-1-(2-chloro-7-(1-methoxyethyl)pyrazolo[1,5-a]pyrimidin-6-yl)-3-(2-(trifluoromethyl)pyridin-4-yl)urea according to claim 17, characterized by an X-ray powder diffraction pattern with peaks at 15.03°±0.2°, 19.93±0.2°, and 24.22±0.2°.

21. A crystalline from of 1-(5-chloro-6-(2H-1,2,3-triazol-2-yl)pyridin-3-yl)-3-(2-chloro-7-isopropylpyrazolo[1,5-a]pyrimidin-6-yl)urea according to claim 17, characterized by an X-ray powder diffraction pattern with peaks at 14.54°±0.2°, 18.24±0.2°, and 21.90±0.2°.

22. A crystalline from of (S)-1-(5-chloro-6-(2H-1,2,3-triazol-2-yl)pyridin-3-yl)-3-(2-chloro-7-(1-methoxyethyl)pyrazolo[1,5-a]pyrimidin-6-yl)urea according to claim 17, characterized by an X-ray powder diffraction pattern with peaks at 12.87°±0.2°, 13.27±0.2°, 27.25°±0.2°, and 29.10±0.2°.
